SEQUENCE LISTING (1 sequence)

SEQ ID NO: 1 - DNA, 36201 bp, synthetic construct
  source 1..36201: mol_type = other DNA
cgccatcatcaataatataccttattttggattgaagccaatatgataatgagggggtggagtttgtgacgtggcgcggggcgtgggaacggggcgggtgacgtagtagtgtggcggaagtgtgatgttgtaagtgtggcggaacacatgtaagcgccggatgtggtaaaagtgacgtttttggtgtgcgccggtgtacacgggaagtgacaattttcgcgcggttttaggcggatgttgtagtaaatttgggcgtaaccaagtaatatttggccattttcgcgggaaaactgaataagaggaagtgaaatctgaataattctgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgcgcagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaaattatgggcagtgggtgatagagtggtgggtttggtgtggtaattttttttttaatttttacagttttgtggtttaaagaattttgtattgtgattttttaaaaggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcctcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggtggtgagacacaagaatcgcctgctactgttgtcttccgtccgcccggcaataataccgacggaggagcaacagcaggaggaagccaggcggcggcggcggcaggagcagagcccatggaacccgagagccggcctggaccctcgggaatgaatgttgtacaggtggctgaactgtttccagaactgagacgcattttaaccattaacgaggatgggcaggggctaaagggggtaaagagggagcggggggcttctgaggctacagaggaggctaggaatctaacttttagcttaatgaccagacaccgtcctgagtgtgttacttttcagcagattaaggataattgcgctaatgagcttgatctgctggcgcagaagtattccataaagcagctgaccacttactggctgcagccaggggatgattttgaggaggctattagggtatatgcaaaggtggcacttaggccagattgcaagtacaagattagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtgaggtttactggtcccaattttagcggtacggttttcctggccaataccaatcttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctgtttgaaaggtgtaccttgggtatcctgtctgagggtaactccagggtgcgccacaatgtggcctccgactgtggttgctttatgctagtgaaaagcgtggctgtgattaagcataacatggtgtgtggcaactgcgaggacagggcctctcagatgctgacctgctcggacggcaactgtcacttgctgaagaccattcacgtagccagccactctcgcaaggcctggccagtgtttgagcacaacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtctcatgtagttttgtatctgttttgcagcagccgccgccatgagcgccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaaccgtttccggggtaggggagatcagctgggaagaaagcaggttcctaagcagctgcgacttaccgcagccggtgggcccgtaaatcacacctattaccggctgcaactggtagttaagagagctgcagctgccgtcatccctgagcaggggggccacttcgttaagcatgtccctgacttgcatgttttccctgaccaaatccgccagaaggcgctcgccgcccagcgatagcagttcttgcaaggaagcaaagtttttcaacggtttgaggccgtccgccgtaggcatgcttttgagcgtttgaccaagcagttccaggcggtcccacagctcggtcacgtgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggttgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttaagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcactaggtgcacgcgccaaccgcggttgtgcagggtgacaaggtcaacgctggtggctacctctccgcgtaggcgctcgttggtccagcagaggcggccgcccttgcgcgagcagaatggcggtagtgggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctancatgtagaactggttgacggcctggtaggcgcagcatcccttttctacgggtagcgcgtatgcctgcgcggccttccggagcgaggtgtgggtgagcgcaaaggtgtccctaaccatgactttgaggtactggtatttgaagtcagtgtcgtcgcatccgccctgctcccagagcaaaaagtccgtgcgctttttggaacgcgggtttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggtccgcggctaggtctcgcgcggcggtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccagttggaggagtggctgttgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagccgggtcagggcgcgggctaggtccaggtgatacctgatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgggcccccggaggtagggggggctcgggacccgccgggagagggggcaggggcacgtcggcgccgcgcgcgggcaggagctggtgctgcgcgcggaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggagatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgccgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccgtgcggcagggatacggcgctaacgatgcatctcaacaattgttgtgtaggtactccgccaccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggccaggtcggcgacaacgcgctcggctaatatggcctgctgcacctgcgtgagggtagactggaagtcgtccatgtccacaaagcggtggtatgcgcccgtgttgatggtgtaagtgcagttggccataacggaccagttaacggtctggtgacccggctgcgagagctcggtgtacctgagacgcgagtaagcccttgagtcaaagacgtagtcgttgcaagtccgcaccaggtactggtatcccaccaaaaagtgcggcggcggctggcggtagaggggccagcgtagggtggccggggctccgggggcgaggtcttccaacataaggcgatgatatccgtagatgtacctggacatccaggtgatgccggcggcggtggtggaggcgcgcggaaagtcacggacgcggttccagatgttgcgcagcggcaaaaagtgctccatggtcgggacgctctggccggtcaggcgcgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggatgctgcgctagcttttttggccactggccgcgcgcggcgtaagcggttaggctggaaagcgaaagcattaagtggctcgctccctgtagccggagggttattttccaagggttgagtcgcgggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagtcgcggccgcgatatcgctagcgaagttcctattctctagaaagtataggaacttcggatcctctagagtcgaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccgccagtgtccaccgtggacgcggccattcagaccgtggtgcgcggagcccggcgctacgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgagccgctcgaaggctggccgcgggtattgtcactgtgccccccaggtccaggcgacgagcggccgccgcagcagccgcggccattagtgttatgactcagggtcgcaggggcaacgtgtactgggtgcgcgactcggttagcggcctgcgcgtgcccgtgcgcacccgccccccgcgcaactagattgcaataaaaaactacttagactcgtactgttgtatgtatccagcggcggcggcgcgcatcgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgatgaacttgacgacgaggtggaactgttgcacgcgaccgcgcccaggcgacgggtacagtggaaaggtcgacgcgtaagacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtgacactgcagcaggtgctgcccgcgcttgcaccgtccgaagaaaagcgcggcctaaagcgcgagtctggtgacttggcacccaccgtgcagctgatggtacccaagcgtcagcgactggaagatgtcttggaaaaaatgaccgtggagcctgggctggagcccgaggtccgcgtgcggccaatcaagcaggtggcaccgggactgggcgtgcagaccgtggacgttcagatacccaccaccagtagcactagtattgccactgccacagagggcatggagacacaaacgtccccggttgcctcggcggtggcagatgccgcggtgcaggcggccgctgcggccgcgtccaagacctctacggaggtgcaaacggacccgtggatgtttcgtgtttcagccccccggcgtccgcgccgttcaaggaagtacggcgccgccagcgcgctactgcccgaatatgccctacatccttccatcgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcgccggtatcctgcccctccttattccactgatcgccgcggcgattggcgccgtgcccggaattgcatccgtggccttgcaggcgcagagacactgattaaaaacaagttacatgtggaaaaatcaaaataaaagtctggactctcacgctcgcttggtcctgtaactattttgtagaatggaagacatcaactttgcgtcactggccccgcgacacggctcgcgcccgttcatgggaaactggcaagatatcggcaccagcaatatgagcggtggcgccttcagctggggctcgctgtggagcggcattaaaaatttcggttccgccgttaagaactatggcagcaaagcctggaacagcagcacaggccagatgctgagggacaagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgacccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgtccgccgttgttgtaacccgtcctagccgcgggtccctgcgccgcgccgccagcggtccgcgatcgttgcggcccgtagccagtggcaactggcaaagcacactgaacagcatcgtgggtttgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagttcgcccgcgccaccgagacgtacttcagcctgaataacaagtttagaaaccccacggtggcgcctacgcacgacgtgaccacagaccggtctcagcgtttgacgctgcggttcatccccgtggaccgcgaggatactgcgtactcgtacaaggcgcggttcaccctagctgtgggtgataaccgtgtgctagacatggcttccacgtactttgacatccgcggcgtgctggacaggggccctacttttaagccctactctggcactgcctacaacgcactggcccccaagggtgcccccaactcgtgcgagtgggaacaaaatgaaactgcacaagtggatgctcaagaacttgacgaagaggagaatgaagccaatgaagctcaggcgcgagaacaggaacaagctaagaaaacccatgtatatgcccaggctccactgtccggaataaaaataactaaagaaggtctacaaataggaactgccgacgccacagtagcaggtgccggcaaagaaattttcgcagacaaaacttttcaacctgaaccacaagtaggagaatctcaatggaacgaagcggatgccacagcagctggtggaagggttcttaaaaagacaactcccatgaaaccctgctatggctcatacgctagacccaccaattccaacggcggacagggcgttatggttgaacaaaatggtaaattggaaagtcaagtcgaaatgcaatttttttccacatccacaaatgccacaaatgaagttaacaatatacaaccaacagttgtattgtacagcgaagatgtaaacatggaaactccagatactcatctttcttataaacctaaaatgggggataaaaatgccaaagtcatgcttggacaacaagcaatgccaaacagaccaaattacattgcttttagagacaattttattggtctcatgtattacaacagcacaggtaacatgggtgtccttgctggtcaggcatcgcagttgaacgctgttgtagatttgcaagacagaaacacagagctgtcctaccagcttttgcttgattcaattggcgacagaacaagatacttttcaatgtggaatcaagctgttgacagctatgatccagatgtcagaattattgagaaccatggaactgaggatgagttgccaaattattgctttcctcttggtggaattgggattactgacacttttcaagctgttaaaacaactgctgctaacggggaccaaggcaatactacctggcaaaaagattcaacatttgcagaacgcaatgaaataggggtgggaaataactttgccatggaaattaacctgaatgccaacctatggagaaatttcctttactccaatattgcgctgtacctgccagacaagctaaaatacaaccccaccaatgtggaaatatctgacaaccccaacacctacgactacatgaacaagcgagtggtggctcctgggcttgtagactgctacattaaccttggggcgcgctggtctctggactacatggacaacgttaatccctttaaccacccccgccatgcgggcctgcgttaccgctccatgttgttgggaaacggccgctacgtgccctttcacattcaggtgccccaaaagttttttgccattaaaaacctcctcctcctgccaggctcatacacatatgaatggaacttcaggaaggatgttaacatggttctgcagagctctctgggaaacgaccttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctatatcccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacttttgactcttctgttagctggccgggcaacgaccgcctgcttactcccaatgagtttgagattaagcgctcagttgacggggagggctataacgtagctcagtgcaacatgacaaaggactggttcctagtgcagatgttggccaactacaatattggctaccagggcttctacattccagaaagctacaaagaccgcatgtactcgttcttcagaaacttccagcccatgagccggcaagtggtggacgatactaaatacaaagattatcagcaggttggaattatccaccagcataacaactcaggcttcgtaggctacctcgctcccaccatgcgcgagggacaagcttaccccgctaatgttccctacccactaataggcaaaaccgcggttgatagtattacccagaaaaagtttctttgcgaccgcaccctgtggcgcatccccttctccagtaactttatgtccatgggtgcgctcacagacctgggccaaaaccttctctacgcaaactccgcccacgcgctagacatgacctttgaggtggatcccatggacgagcccacccttctttatgttttgtttgaagtctttgacgtggtccgtgtgcaccagccgcaccgcggcgtcatcgagaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgcttcccaggctttgtttccccacacaagctcgcctgcgccatagttaacacggccggtcgcgagactgggggcgtacactggatggcctttgcctggaacccgcgctcaaaaacatgctacctctttgagccctttggcttttctgaccaacgtctcaagcaggtttaccagtttgagtacgagtcactcctgcgccgtagcgccattgcctcttcccccgaccgctgtataacgctggaaaagtccacccaaagcgtgcaggggcccaactcggccgcctgtggcctattctgctgcatgtttctccacgcctttgccaactggccccaaactcccatggatcacaaccccaccatgaaccttattaccggggtacccaactccatgcttaacagtccccaggtacagcccaccctgcgccgcaaccaggaacagctctacagcttcctggagcgccactcgccctacttccgcagccacagtgcgcaaattaggagcgccacttctttttgtcacttgaaaaacatgtaaaaataatgtactaggagacactttcaataaaggcaaatgtttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttacagcactggaacactatcagcgccgggtggtgcacgctggccagcacgctcttgtcggagatcanatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccagtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcatccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgagacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaagatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaagcgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctgtgctacacctggcaaacggccatgggcgtgtggcagcagtgcctggaggagcgcaacctgaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttntgcagctagccaactaccttgcctaccactccgacatcatggaagacgtgagcggtgacggcctactggagtgtcactgtcgctgcaacctatgcaccccgcaccgctccctggtctgcaattcacaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccattaacaaagcccgccaagagtttctgctacgaaagggacggggggtttacttggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaggaagcttccgaggccgaagaggtgtcagacgaaacaccgtcaccctcggtcgcattcccctcgccggcgccccagaaatcggcaaccgttcccagcattgctacaacctccgctcctcaggcgccgccggcactgcccgttcgccgacccaaccgtagatgggacaccactggaaccagggccggtaagtctaagcagccgccgccgttagcccaagagcaacaacagcgccaaggctaccgctcgtggcgcgtgcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcaacagcagcggccacgcagaagcaaaggcgaccggatagcaagactctgacaaagcccaagaaatccacagcggcggcagcagcaggaggaggagcactgcgtctggcgcccaacgaacccgtatcgacccgcgagcttagaaacaggatttttcccactctgtatgctatatttcaacagagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgaaaatcagagggcgaggtattcagctcaacgacgagtcggtgagctcctctcttggtctccgtccggacgggacatttcagatcggcggcgctggccgctcttcatttacgccccgtcaggcgatcctaactctgcagacctcgtcctcggagccgcgctccggaggcattggaactctacaatttattgaggagttcgtgccttcggtttacttcaaccccttttctggacctcccggccactacccggaccagtttattcccaactttgacgcggtaaaagactcggcggacggctacgactgacagatctgagctcgcggccgcgatatcgctagcgaagttcctattctctagaaagtataggaacttcgatcctctagagtcgacctgcaggcatgcaagcttggcactgcaataaattacttacttaaaatcagtcagcaaatctttgtccagcttattcagcatcacctcctttccctcctcccaactctggtatttcagcagccttttagctgcgaactttctccaaagtctaaatgggatgtcaaattcctcatgttcttgtccctccgcacccactatcttcatattgttgcagatgaaacgcgccagaccgtctgaagacaccttcaaccctgtgtacccatatgacacggaaaccggccctccaactgtgcctttccttacccctccctttgtgtcgccaaatgggttccaagaaagtccccccggagtgctttctttgcgtctttcagaacctttggttacctcacacggcatgcttgcgctaaaaatgggcagcggcctgtccctggatcaggcaggcaaccttacatcaaatacaatcactgtttctcaaccgctaaaaaaaacaaagtccaatataactttggaaacatccgcgccccttacagtcagctcaggcgccctaaccatggccacaacttcgcctttggtggtctctgacaacactcttaccatgcaatcacaagcaccgctaaccgtgcaagactcaaaacttagcattgctaccaaagagccacttacagtgttagatggaaaactggccctgcagacatcagcccccctctctgccactgataacaacgccctcactatcactgcctcacctcctcttactactgcaaatggtagtctggctgttaccatggaaaacccactttacaacaacaatggaaaacttgggctcaaaattggcggtcctttgcaagtggccaccgactcacatgcactaacactaggtactggtcagggggttgcagttcataacaatttgctacatacaaaagttacaggcgcaatagggtttgatacatctggcaacatggaacttaaaactggagatggcctctatgtggatagcgccggtcctaaccaaaaactacatattaatctaaataccacaaaaggccttgcttttgacaacaccgcaataacaattaacgctggaaaagggttggaatttgaaacagactcctcaaacggaaatcccataaaaacaaaaattggatcaggcatacaatataataccaatggagctatggttgcaaaacttggaacaggcctcagttttgacagctccggagccataacaatgggcagcataaacaatgacagacttactctttggacaacaccagacccatccccaaattgcagaattgcttcagataaagactgcaagctaactctggcgctaacaaaatgtggcagtcaaattttgggcactgtttcagctttggcagtatcaggtaatatggcctccatcaatggaactctaagcagtgtaaacttggttcttagatttgatgacaacggagtgcttatgtcaaattcatcactggacaaacagtattggaactttagaaacggggactccactaacggtcaaccatacacttatgctgttgggtttatgccaaacctaaaagcttacccaaaaactcaaagtaaaactgcaaaaagtaatattgttagccaggtgtatcttaatggtgacaagtctaaaccattgcattttactattacgctaaatggaacagatgaaaccaaccaagtaagcaaatactcaatatcattcagttggtcctggaacagtggacaatacactaatgacaaatttgccaccaattcctataccttctcctacattgcccaggaataaagaatcgtgaacctgttgcatgttatgtttcaacgtgtttatttttcaattcgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccaccatgtttgtctttctcgtgctgctgcccctcgtgagcagccagtgcgtcaatctgacaacaaggacccagctgccccccgcctacaccaactccttcacaagaggcgtgtattaccccgataaggtcttcagatccagcgtcctccacagcacccaagatttgtttctgcctttcttcagcaacgtgacatggttccacgccattcatgtcagcggcacaaacggcacaaagaggtttgacaaccccgtgctccccttcaacgacggcgtgtacttcgccagcacagagaaatccaatatcattaggggctggatcttcggcacaacactggattccaagacccagtctctgctcattgtgaataacgccaccaacgtggtgattaaggtctgtgagtttcagttctgcaacgacccctttctgggagtctactaccacaagaataataagagctggatggagtccgagtttagggtgtacagctccgccaacaactgtaccttcgaatacgtgtcccagcctttcctcatggatctggagggcaagcaaggcaatttcaaaaatctgagagagttcgtgttcaaaaacattgatggatacttcaaaatctacagcaagcatacccccattaatctggtgagggatctgccccaaggattctccgctctggaacctctggtggatctgcccattggcattaacatcacaagattccagaccctcctcgccctccatagatcctatctgacccccggcgactcctccagcggatggacagccggagctgccgcctactacgtgggctatctgcagccaagaacctttctgctgaagtacaacgagaacggcaccatcacagacgctgtcgattgcgctctcgaccctctgagcgagaccaaatgcacactgaagagcttcaccgtggaaaagggcatctatcagaccagcaacttcagagtgcagcctaccgagagcattgtgaggtttcccaacatcaccaatctgtgtcctttcggcgaggtctttaatgccacaaggttcgcttccgtgtatgcttggaataggaagaggatcagcaattgcgtcgccgactattccgtcctctataacagcgcctccttctccaccttcaaatgttatggcgtgtcccccaccaagctcaacgacctctgcttcaccaatgtgtacgctgactccttcgtcattaggggcgacgaggtgaggcaaattgcccccggccagaccggcaagattgctgattacaactacaaactgcccgacgattttaccggctgcgtgatcgcttggaactccaacaatctggactccaaagtgggcggaaactacaattacctctacagactctttagaaaaagcaatctgaagcccttcgagagagacatctccaccgaaatctaccaagccggaagcacaccttgcaatggcgtcgagggatttaactgctacttccctctgcagagctacggctttcaacctaccaacggcgtcggatatcaaccctatagggtggtcgtgctgagctttgaactgctgcatgctcccgccaccgtctgcggacctaagaagagcaccaatctcgtcaaaaacaagtgcgtgaacttcaacttcaatggactgaccggcaccggcgtgctgaccgagagcaataagaagtttctgcccttccagcagttcggaagggatattgccgataccacagatgctgtgagggacccccaaaccctcgagattctggatatcaccccttgcagcttcggaggagtgtccgtgatcacccccggaacaaacacctccaatcaagtggctgtgctgtaccaagacgtgaactgcacagaagtccccgtggccatccatgccgaccagctgacccctacatggagagtgtactccaccggcagcaatgtgttccagacaagagccggatgcctcattggagctgaacacgtcaacaacagctacgagtgcgacattcccatcggcgccggcatttgtgcctcctatcagacccagaccaacagcccaagaagggctagaagcgtcgcttcccaatccatcattgcctacaccatgtctctgggagccgaaaactccgtcgcctactccaacaatagcatcgccatccccaccaattttaccatctccgtgaccacagagattctgcccgtgtccatgacaaagacatccgtggactgcaccatgtacatctgtggcgacagcaccgagtgtagcaatctgctgctgcaatatggcagcttctgcacccagctgaacagagccctcaccggcatcgccgtcgaacaagacaagaacacccaagaggtgttcgcccaagtgaagcaaatctacaagaccccccctatcaaagatttcggaggattcaactttagccagattctgcccgatcctagcaagccttccaagaggagcttcatcgaggatctgctgtttaataaggtgacactggccgacgctggcttcattaaacagtacggcgattgtctgggcgacatcgctgctagggatctgatctgcgctcagaagttcaacggactgacagtcctccctcctctgctgaccgacgagatgatcgctcagtataccagcgctctgctggctggaaccattaccagcggctggacattcggcgctggagccgccctccaaattccctttgccatgcagatggcctatagattcaacggcattggcgtcacccaaaatgtgctgtatgaaaatcagaagctgattgctaaccaattcaatagcgccattggcaagatccaagactctctgagctccacagccagcgccctcggaaagctgcaagacgtggtgaatcaaaacgcccaagctctgaacacactggtgaaacagctcagcagcaactttggagccatcagcagcgtgctcaatgatatcctctctaggctggacaaagtggaggccgaagtccagatcgatagactcatcaccggcagactccaatctctgcagacatacgtcacccaacagctcattagagctgccgaaatcagagcctccgccaatctggccgccaccaagatgtccgagtgcgtgctgggacagagcaagagagtggacttctgtggcaagggataccatctgatgagcttcccccagagcgctccccatggagtggtctttctgcatgtcacatacgtgcccgcccaagagaagaacttcaccaccgctcccgccatttgccacgatggaaaggcccactttcccagagaaggagtgttcgtgagcaacggcacacactggtttgtcacccagagaaatttttacgagccccagattatcaccaccgacaacaccttcgtgtccggaaactgcgatgtcgtgattggcatcgtgaacaacacagtctacgaccctctgcagcccgaactcgacagcttcaaggaagagctggacaagtacttcaagaatcacacatcccccgacgtggatctgggcgacattagcggcattaatgcctccgtcgtcaacattcagaaggagattgatagactgaatgaagtcgccaagaacctcaatgagtctctgattgatctgcaagagctgggcaagtacgagcaatacatcaaatggccttggtacatctggctgggattcatcgctggactcatcgccatcgtgatggtcaccattatgctgtgttgcatgaccagctgctgcagctgtctgaagggctgctgcagctgcggaagctgctgcaagtttgacgaagacgactccgagcccgtgctgaagggcgtcaagctgcattatacataaactagtgctggaattcgcccttatagagtgctggaattcgcccttatagagtgctggaattcgcccttatatctagtaacggccgccagtgtgctggaattcgcccttataacttcgtatagcatacattatacgaagttattgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaataacttcgtatagcatacattatacgaagttataagggcgaattctgcagatatccatcctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacaacgtgtttatttttcaattgcagaaagaattgcagaaaatttcaagtcatttttcattcagtagtatagccccaccaccacatagcttatactaatcaccgtaccttaatcaaactcacagaaccctagtattcaacctgccacctccctcccaacacacagagtacacagtcctttctccccggctggccttaaacagcatcatatcatgggtaacagacatattcttaggtgttatattccacacggtctcctgtcgagccaaacgctcatcagtgatgttaataaactccccgggcagctcgcttaagttcatgtcgctgtccagctgctgagccacaggctgctgtccaacttgcggttgctcaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaagtcagcacagtaactgcagcacagtaccacaatattgtttaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgtgtctctgtctcaaaaggaggtaggcgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgtcgcttagctcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaactgccctcacgtccaagtggacgtaaaggctaaacccttcanggtgaatctcctctataaacattccagcaccttcaaccatgcccaaataattttcatctcgccaccttatcaatatgtctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtactgctcaaaaaatcaggcaaagcctcgcgcaaaaaagcaagcacatcgtagtcatgctcatgcagataaaggcaggtaagttccggaaccaccacagaaaaagacaccatttttctctcaaacatgtctgcgggttcctgcataaacacaaaataaaataacaaaaaaaaaaaaacatttaaacattagaagcctgtcttacaacaggaaaaacaacccttataagcataagacggactacggccatgccggcgtgaccgtaaaaaaactggtcaccgtgattaaaaagcaccaccgacagttcctcggtcatgtccggagtcataatgtaagactcggtaaacacatcaggttggttaacatcggtcagtgctaaaaagcgaccgaaatagcccgggggaatacatacccgcaggcgtagagacaacattacagcccccataggaggtataacaaaattaataggagagaaaaacacataaacccctgaaaaaccctcctgcccctaggcaaaatagcaccctcccgctccagaacaacatacagcgcttccacagcggcagccataacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaccacccagaaaaccgcacgcgaacctacgcccagaaacgaaagccaaaaaacccacaacttcctcaaatcttcacttccgttttcccacgatacgtcacttcccattttaaaaaaaaactacaattcccaatacatgcaagttactccgccctaaaacctacgtcacccgccccgttcccacgccccgcgccacgtcacaaactccaccccctcattatcatattggcttcaatccaaaataaggtatattattgatgatggcgat

(19) United States
(12) Patent Application Publication
(10) Pub. No.: US 2026/0335430 A1
(43) Pub. Date: Oct. 29, 2026

(54) METHODS AND MATERIALS FOR USING ADENOVIRUS VECTORS TO IMMUNIZE MAMMALS

(71) Applicants: Mayo Foundation for Medical Education and Research, Rochester, MN (US); Moat Biotechnology Corporation, Oklahoma City, OK (US)

(72) Inventors: Michael A. Barry, Rochester, MN (US); Russell Rother, Steamboat Springs, CO (US)

(21) Appl. No.: 19/113,058

(22) PCT Filed: Sep. 19, 2023
(86) PCT No.: PCT/US2023/033145

§ 371 Date: Mar. 19, 2025

Related U.S. Application Data
(60) Provisional application No. 63/407,984, filed on Sep. 19, 2022.

(51) Int. Cl. A61K 39/215; A61K 39/00; A61K 39/39; A61P 31/14; C12N 7/00; C12N 15/861

(52) U.S. Cl. CPC A61K 39/215; A61K 39/39; A61P 31/14; C12N 7/00; C12N 15/861; A61K 2039/53; A61K 2039/543; A61K 2039/545; A61K 2039/55516; A61K 2039/575; C12N 2710/10043; C12N 2770/20034

(57) ABSTRACT

This document provides methods and materials related to using adenovirus vectors to immunize mammals (e.g., humans). For example, methods and materials for intranasally administering adenovirus vectors (e.g., single-cycle adenovirus vectors) as a heterologous vaccination boost to induce IgG, IgA, and T cell immune responses within a mammal (e.g., a human) that received at least one prior vaccine (e.g., one or more prior mRNA-based vaccines) against the targeted pathogen are provided.

### CROSS-REFERENCE TO RELATED APPLICATIONS

This application claims the benefit of U.S. Patent Application Ser. No. 63/407,984, filed on Sep. 19, 2022. The disclosure of the prior application is considered part of, and is incorporated by reference in, the disclosure of this application.

### SEQUENCE LISTING

This application contains a Sequence Listing that has been submitted electronically as an XML file named “07039-2163WO1_SL.xml.” The XML file, created on Sep. 15, 2023, is 38000 bytes in size. The material in the XML file is hereby incorporated by reference in its entirety.

### TECHNICAL FIELD

This document relates to methods and materials for using adenovirus vectors to immunize mammals. For example, adenovirus vectors can be used to intranasally deliver one or more immunogens (e.g., one or more immunogens associated with a pathogen causing an infection) to cells within a mammal (e.g., a human) such that the mammal produces an effective IgG, IgA, and T cell immune response against the immunogen(s).

### BACKGROUND INFORMATION

An infectious disease caused by a coronavirus known as COVID-19 was first reported to the World Health Organization (WHO) Country Office in China on Dec. 31, 2019. As of Jun. 3, 2020, approximately 612,200,000 confirmed cases of COVID-19, including 6,500,000 deaths, have been reported to the WHO (covid19.who.int/).

### SUMMARY

This document provides methods and materials for using adenovirus vectors to immunize mammals. For example, this document provides methods and materials for using adenovirus vectors to deliver nucleic acid intranasally to cells within a mammal (e.g., a human). This document also provides methods and materials for using adenovirus vectors to induce IgG IgA, and T cell immune responses within a mammal (e.g., a human). The adenovirus vectors may be used as a vaccination boost to a prior vaccine, in a manner that triggers IgG, IgA, and T cell immune responses within the recipient. In some cases, adenovirus vectors (e.g., single-cycle adenovirus (SC-Ad) vectors) described herein can be used as a heterologous vaccination boost to deliver nucleic acid encoding one or more (e.g., one, two, three, four, five, six, seven, eight, nine, ten, or more) immunogens intranasally in a manner that triggers IgG, IgA, and T cell immune responses within a mammal (e.g., a human) that received at least one prior vaccine (e.g., one or more prior mRNA vaccines) against the targeted pathogen.

As demonstrated herein, SC-Ads engineered to express one or more immunogens derived from a coronavirus (e.g., SARS-CoV-2) can be administered intranasally to mammals (e.g., humans) who received a prior mRNA-based or DNA-based SARS-CoV-2 vaccine (e.g., a Pfizer® vaccine such as Comirnaty® or an AstraZeneca® vaccine such as Vaxzevria®) to induce effective IgG, IgA, and T cell immune responses against the coronavirus. For example, intranasal administration of SC-Ads designed to express a SARS-CoV-2 polypeptide such as a spike polypeptide (or a fragment thereof) as a heterologous boost to a human who received a prior mRNA-based or DNA-based SARS-CoV-2 vaccine (e.g., a Pfizer® vaccine such as Comirnaty® or an AstraZeneca® vaccine such as Vaxzevria®) can induce the human's immune system to produce IgG antibodies that can bind to and neutralize the coronavirus target, to produce IgA antibodies that can bind to and neutralize the coronavirus target, and/or Th1 T cell immune responses targeted against the coronavirus target.

Having the ability to perform heterologous vaccination boosts produce immune responses effectively against viral and/or bacterial pathogens (e.g., a coronavirus) in mammals (e.g., humans) can improve survival and minimize the impact of the infection. Adenovirus vectors encoding one or more immunogens can be used to provide a mammal with sustained, long-term immunity against an infectious pathogen (e.g., a coronavirus). For example, adenovirus vectors encoding one or more immunogens associated with COVID-19 (e.g., one or more immunogens derived from SARS-CoV-2) can be used as a robust heterologous vaccination boost in the COVID-19 pandemic to generate humoral immunity against infections.

In general, one aspect of this document features methods for providing a human who received a prior coronavirus vaccine with a heterologous vaccination boost. The methods can include, or consist essentially of, intranasally administering a SC-Ad to a human, where the SC-Ad includes a genome lacking at least a portion of a nucleic acid sequence that encodes an adenovirus polypeptide, where the SC-Ad includes the adenovirus polypeptide, where the SC-Ad includes a nucleic acid sequence encoding an immunogen of a coronavirus, and where the prior coronavirus vaccine did not comprise the SC-Ad. This aspect of the invention also provides a SC-Ad comprising a genome lacking at least a portion of a nucleic acid sequence that encodes an adenovirus polypeptide, where the SC-Ad includes the adenovirus polypeptide, and where the SC-Ad includes a nucleic acid sequence encoding an immunogen of a coronavirus, for use in a method of providing a human, who received a prior coronavirus vaccine that did not comprise the SC-Ad, with a heterologous vaccination boost, the method comprising intranasally administering the SC-Ad to the human. The adenovirus polypeptide can be a fiber polypeptide, a V polypeptide, a hexon polypeptide, a penton base polypeptide, or a pIIIa polypeptide. The immunogen can include a coronavirus Spike polypeptide or an immunogenic fragment thereof. The immunogen can consist of or consist essentially of an amino acid sequence set forth in any one of SEQ ID NOs:1-4 of International PCT Patent Publication No. WO 2022/040204. The immunogen can consist of or consist essentially of an amino acid sequence set forth in any one of SEQ ID NOs: 12, 42, 43, 44, 47, and 48 of International PCT Patent Publication No. WO 2022/040204. The SC-Ad also can include a nucleic acid sequence encoding an adjuvant polypeptide. The adjuvant polypeptide can be a granulocyte-macrophage colony-stimulating factor (GM-CSF) polypeptide, an interleukin 4 (IL-4) polypeptide, an interleukin 21 (IL-21) polypeptide, a CD40 ligand (CD40L) polypeptide, a 4-1BB ligand (4-1BBL) polypeptide, a transforming growth factor beta (TGF-β) polypeptide, a Clostridium difficile TcdA polypeptide, a C. difficile TcdB polypeptide, or biologically active fragments thereof. The coronavirus Spike polypeptide can be fused to the adjuvant polypeptide. The coronavirus Spike polypeptide fused to the adjuvant polypeptide can consist essentially of or consist of an amino acid sequence set forth in SEQ ID NO:5 of International PCT Patent Publication No. WO 2022/040204. The SC-Ad also can include a nucleic acid sequence encoding a chaff polypeptide. The chaff polypeptide can be a fragment of an ACE2 polypeptide. The fragment of an ACE2 polypeptide can include the extracellular region of an ACE2 polypeptide and can lack a transmembrane domain. The chaff polypeptide can consist essentially of or consist of an amino acid sequence set forth in SEQ ID NO:8 of International PCT Patent Publication No. WO 2022/040204 or SEQ ID NO:9 of International PCT Patent Publication No. WO 2022/040204. The coronavirus Spike polypeptide can be fused to the chaff polypeptide. The coronavirus can be a betacoronavirus. The betacoronavirus can be SARS-CoV-2. The human can be a human who received the prior coronavirus vaccine at least 90 days before the intranasally administering. The human can be a human who received the prior coronavirus vaccine at least 120 days before the intranasally administering. The human can be a human who received the prior coronavirus vaccine at least 150 days before the intranasally administering. The human can be a human who received the prior coronavirus vaccine at least 180 days before the intranasally administering. The prior coronavirus vaccine can have been an mRNA-based vaccine. The SC-Ad can infect a cell of the human, and expression of the immunogen in the cell can lead to an increase in the amount of serum IgG antibodies that bind to the immunogen. The SC-Ad can infect a cell of the human, and expression of the immunogen in the cell can lead to an increase in the amount of mucosal IgA antibodies that bind to the immunogen. The SC-Ad can infect a cell of the human, and expression of the immunogen in the cell can lead to an increase in the amount of neutralizing antibodies in the human that bind to the immunogen. The SC-Ad can infect a cell of the human, and expression of the immunogen in the cell can lead to an increase in the number of Th1 T cells within the human that target the immunogen.

In another aspect, this document features methods for providing a human who received a prior vaccine against a virus causing respiratory disease with a vaccination boost, where the method comprises intranasally administering a SC-Ad to the human, where the SC-Ad comprises a genome lacking at least a portion of a nucleic acid sequence that encodes an adenovirus polypeptide, where the SC-Ad comprises the adenovirus polypeptide, and where the SC-Ad comprises a nucleic acid sequence encoding an immunogen of the virus causing respiratory disease. This aspect of the invention also provides a SC-Ad comprising a genome lacking at least a portion of a nucleic acid sequence that encodes an adenovirus polypeptide, where the SC-Ad comprises the adenovirus polypeptide, and where the SC-Ad comprises a nucleic acid sequence encoding an immunogen of a virus causing respiratory disease, for use in a method of providing a human who received a prior vaccine against the virus causing respiratory disease with a vaccination boost, where the method comprises intranasally administering the SC-Ad to the human. Except for where context requires otherwise, a SC-Ad for use in such methods or medical uses may be substantially or entirely as described in connection with the other aspects of the invention, or any embodiment thereof. The benefits of such methods and medical uses may be as described elsewhere in the present disclosure. The virus causing the respiratory disease can be a coronavirus; an influenza virus; a respiratory syncytial virus (RSV); a metapneumovirus (MPV); a rhinovirus; a bocavirus; or a parainfluenza virus (PIV). The immunogen of the virus causing the respiratory disease can be a coronavirus Spike polypeptide or an immunogenic fragment thereof, an influenza haemagglutinin polypeptide; or an immunogenic fragment thereof; an RSV fusion (F) protein polypeptide, or an immunogenic fragment thereof; an MPV fusion (F) protein polypeptide, or an immunogenic fragment thereof, rhinovirus VP1 protein polypeptide, or an immunogenic fragment thereof; or a PIV fusion (F) protein polypeptide, or an immunogenic fragment thereof. The immunogen can include a coronavirus Spike polypeptide or an immunogenic fragment thereof. The immunogen can consist of or consist essentially of an amino acid sequence set forth in any one of SEQ ID NOs:1-4 of International PCT Patent Publication No. WO 2022/040204. The immunogen can consist of or consist essentially of an amino acid sequence set forth in any one of SEQ ID NOs: 12, 42, 43, 44, 47, and 48 of International PCT Patent Publication No. WO 2022/040204. The SC-Ad can include a nucleic acid sequence encoding an adjuvant polypeptide. The adjuvant polypeptide can be a GM-CSF polypeptide, an IL-4 polypeptide, an IL-21 polypeptide, a CD40L polypeptide, a 4-1BBL polypeptide, a TGF-β polypeptide, a Clostridium difficile TcdA polypeptide, a C. difficile TcdB polypeptide, or biologically active fragments thereof. The immunogen can be fused to the adjuvant polypeptide. The immunogen fused to the adjuvant polypeptide can consist essentially of or consist of an amino acid sequence set forth in SEQ ID NO:5 of International PCT Patent Publication No. WO 2022/040204. The SC-Ad further can include a nucleic acid sequence encoding a chaff polypeptide. The chaff polypeptide can be a fragment of an ACE2 polypeptide. The fragment of an ACE2 polypeptide can include the extracellular region of an ACE2 polypeptide and can lack a transmembrane domain. The chaff polypeptide can consist essentially of or consist of an amino acid sequence set forth in SEQ ID NO:8 of International PCT Patent Publication No. WO 2022/040204 or SEQ ID NO:9 of International PCT Patent Publication No. WO 2022/040204. The immunogen can be fused to the chaff polypeptide. The human can be a human who received the prior coronavirus vaccine at least 90 days before the intranasally administering. The human can be a human who received the prior coronavirus vaccine at least 120 days before the intranasally administering. The human can be a human who received the prior coronavirus vaccine at least 150 days before the intranasally administering. The human can be a human who received the prior coronavirus vaccine at least 180 days before the intranasally administering. The prior vaccine can have been an mRNA-based vaccine. The SC-Ad can infect a cell of the human, and expression of the immunogen in the cell can lead to an increase in the amount of serum IgG antibodies that bind to the immunogen. The SC-Ad can infect a cell of the human, and expression of the immunogen in the cell can lead to an increase in the amount of mucosal IgA antibodies that bind to the immunogen. The SC-Ad can infect a cell of the human, and expression of the immunogen in the cell can lead to an increase in the amount of neutralizing antibodies in the human that bind to the immunogen. The SC-Ad can infect a cell of the human, and expression of the immunogen in the cell can lead to an increase in the number of Th1 T cells within the human that target the immunogen.

In another aspect, this document features methods for providing a mammal who received a prior vaccine targeting a pathogen with a vaccination boost against the pathogen, where the method comprises intranasally administering a SC-Ad to the mammal, where the SC-Ad comprises a genome lacking at least a portion of a nucleic acid sequence that encodes an adenovirus polypeptide, where the SC-Ad comprises the adenovirus polypeptide, where the SC-Ad comprises a nucleic acid sequence encoding an immunogen of the pathogen. This aspect of the invention also provides a SC-Ad comprising a genome lacking at least a portion of a nucleic acid sequence that encodes an adenovirus polypeptide, where the SC-Ad comprises the adenovirus polypeptide, where the SC-Ad comprises a nucleic acid sequence encoding an immunogen of a pathogen, for use in a method of providing a mammal who received a prior vaccine targeting a pathogen with a vaccination boost against the pathogen. Except for where context requires otherwise, a SC-Ad for use in this aspect of the invention may be substantially or entirely as described in connection with the other aspects of the invention, or any embodiment thereof. The benefits of such methods and medical uses may be as described elsewhere in the present disclosure. The mammal can be a human. The pathogen can cause a respiratory disease. The pathogen causing the respiratory disease can be a coronavirus; an influenza virus; a RSV; a MPV; a rhinovirus; a bocavirus; or a PIV. The adenovirus polypeptide can be a fiber polypeptide, a V polypeptide, a hexon polypeptide, a penton base polypeptide, or a pIIIa polypeptide. The mammal can be a mammal that received the prior vaccine at least 90 days before the intranasally administering. The mammal can be a mammal that received the prior vaccine at least 120 days before the intranasally administering. The mammal can be a mammal that received the prior vaccine at least 150 days before the intranasally administering. The mammal can be a mammal that received the prior vaccine at least 180 days before the intranasally administering. The prior vaccine can have been an mRNA-based vaccine. The SC-Ad can infect a cell of the mammal, and expression of the immunogen in the cell can lead to an increase in the amount of serum IgG antibodies that bind to the immunogen. The SC-Ad can infect a cell of the mammal, and expression of the immunogen in the cell can lead to an increase in the amount of mucosal IgA antibodies that bind to the immunogen. The SC-Ad can infect a cell of the mammal, and expression of the immunogen in the cell can lead to an increase in the amount of neutralizing antibodies in the human that bind to the immunogen. The SC-Ad can infect a cell of the mammal, and expression of the immunogen in the cell can lead to an increase in the number of Th1 T cells within the mammal that target the immunogen.

In another aspect, this document features methods for providing a mammal who received a prior vaccine targeting a pathogen with a heterologous vaccination boost against the pathogen. The methods can include, or consist essentially of, intranasally administering a SC-Ad to a mammal, where the SC-Ad includes a genome lacking at least a portion of a nucleic acid sequence that encodes an adenovirus polypeptide, where the SC-Ad includes the adenovirus polypeptide, where the SC-Ad includes a nucleic acid sequence encoding an immunogen of the pathogen, and where the prior vaccine did not include the SC-Ad. This aspect of the invention also provides a SC-Ad comprising a genome lacking at least a portion of a nucleic acid sequence that encodes an adenovirus polypeptide, where the SC-Ad includes the adenovirus polypeptide, where the SC-Ad includes a nucleic acid sequence encoding an immunogen of a pathogen, for use in a method of providing a mammal, who received a prior vaccine that did not include the SC-Ad, with a heterologous vaccination boost against the pathogen. The mammal can be a human. The adenovirus polypeptide can be a fiber polypeptide, a V polypeptide, a hexon polypeptide, a penton base polypeptide, or a pIIIa polypeptide. The pathogen can be a coronavirus. The coronavirus can be a betacoronavirus. The betacoronavirus can be SARS-CoV-2. The immunogen can include a coronavirus Spike polypeptide or an immunogenic fragment thereof. The mammal can be a mammal that received the prior vaccine at least 90 days before the intranasally administering. The mammal can be a mammal that received the prior vaccine at least 120 days before the intranasally administering. The mammal can be a mammal that received the prior vaccine at least 150 days before the intranasally administering. The mammal can be a mammal that received the prior vaccine at least 180 days before the intranasally administering. The prior vaccine can have been an mRNA-based vaccine. The SC-Ad can infect a cell of the mammal, and expression of the immunogen in the cell can lead to an increase in the amount of serum IgG antibodies that bind to the immunogen. The SC-Ad can infect a cell of the mammal, and expression of the immunogen in the cell can lead to an increase in the amount of mucosal IgA antibodies that bind to the immunogen. The SC-Ad can infect a cell of the mammal, and expression of the immunogen in the cell can lead to an increase in the amount of neutralizing antibodies in the human that bind to the immunogen. The SC-Ad can infect a cell of the mammal, and expression of the immunogen in the cell can lead to an increase in the number of Th1 T cells within the mammal that target the immunogen.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagrammatic representation of dose administration and follow-up assessment schema.

FIG. 2 is a table providing the human subject number and number of days since the indicated final COVID-19 vaccination prior to the intranasal administration of a heterologous SC-Ad6-1 boost.

FIG. 3 is a bar graph plotting the fold increase over previous COVID-19 vaccine baseline of anti-coronavirus spike protein IgG antibodies detected in serum from humans on the indicated days following an intranasal administration of a heterologous SC-Ad6-1 boost. Strong anti-spike IgG immune responses induced by the intranasal heterologous boost were detected.

FIG. 4 is a bar graph plotting the fold increase of anti-coronavirus spike protein IgA antibodies detected in nasal mucosal secretions from humans on the indicated days following an intranasal administration of a heterologous SC-Ad6-1 boost. Strong anti-spike IgA immune responses induced by the intranasal heterologous boost were detected.

FIG. 5 is a bar graph plotting the fold increase of live SARS-CoV-2 neutralization by antibodies in serum from humans on the indicated days following an intranasal administration of a heterologous SC-Ad6-1 boost. Strong anti-SARS-CoV-2 neutralizing immune responses induced by the intranasal heterologous boost were detected.

FIG. 6 is a bar graph plotting the spot forming units (SFUs) per million peripheral blood mononuclear cells (PBMCs) for IFN-γ from humans on the indicated days following an intranasal administration of a heterologous SC-Ad6-1 boost. The T cell immune responses were strongly skewed to Th1 (critical for pathogen immunity) which was induced by the intranasal heterologous boost.

FIG. 7 is a bar graph plotting SFUs per million PBMCs for IL-5 from humans on the indicated days following an intranasal administration of a heterologous SC-Ad6-1 boost. The T cell immune responses were skewed toward Th1 (IFN-γ), not Th2 (IL-5), which was induced by the intranasal heterologous boost were detected.

FIG. 8 is a schematic of genomic map of SC-Ad6-1.

### DETAILED DESCRIPTION

This document provides methods and materials for using adenovirus vectors to immunize mammals. In some cases, adenovirus vectors provided herein can be used to deliver intranasally nucleic acid encoding one or more (e.g., one, two, three, four, five, six, seven, eight, nine, ten, or more) immunogens to a mammal (e.g., a human) such that the mammal produces effective immune responses (e.g., IgG, IgA, and T cell immune responses) against those immunogen(s). In some embodiments, the adenovirus vectors can be used to provide a vaccination boost. For example, adenovirus vectors (e.g., single-cycle adenovirus (SC-Ad) vectors) described herein can be used as a heterologous boost to deliver nucleic acid encoding one or more (e.g., one, two, three, four, five, six, seven, eight, nine, ten, or more) immunogens intranasally in a manner that triggers IgG, IgA, and T cell immune responses within a mammal (e.g., a human) that received at least one prior vaccine (e.g., one or more prior mRNA-based and/or DNA-based vaccines) against the targeted pathogen.

Except for where the context requires otherwise, the adenovirus vectors described herein may be used in any of the aspects of the present invention.

An adenovirus vector provided herein (e.g., an adenovirus vector encoding one or more immunogens) can be derived from any adenovirus. An adenovirus used to create an adenovirus vector provided herein can be any appropriate serotype (e.g., Ad1-Ad57). In some cases, an adenovirus can be a replication competent adenovirus. In some cases, an adenovirus can be a replication defective adenovirus. In some cases, an adenovirus can be capable of infecting a human cell (e.g., can be a human adenovirus). In some cases, an adenovirus can be capable of infection a non-human cell (e.g., can be a non-human adenovirus) such as a chimpanzee cell. Examples of adenoviruses that can be used to make an adenovirus vector provided herein include, without limitation, Ad5 adenoviruses, Ad6 adenoviruses, ChAdOx1, and ChAdOx2.

In some cases, an adenovirus vector provided herein (e.g., an adenovirus vector encoding one or more immunogens) can be a SC-Ad. A SC-Ad can have a genome which lacks all or a portion of at least of one of the following adenovirus nucleic acid sequences: fiber protein-encoding sequence, V protein-encoding sequence, hexon-encoding sequence, penton base-encoding sequence (also referred to as a pIII-encoding sequence), VA RNA-encoding sequence, pIIIa protein-encoding sequence (also referred to as a minor capsid protein-encoding sequence), or other early or late gene product-encoding sequences. Examples of nucleic acid sequences that encode adenoviral polypeptides include, without limitation, those set forth in GenBank gi numbers 209842, 58478, or 2935210, and/or annotated in GenBank accession numbers M73260, X17016, or AF030154. In some cases, a deletion of all or a portion of the nucleic acid encoding one or more of the following polypeptides can be engineered into a nucleic acid encoding an adenovirus such that the adenovirus vector does not encode that full-length adenovirus polypeptide or a fully functional version of that adenovirus polypeptide: fiber protein-encoding sequence, V protein-encoding sequence, hexon-encoding sequence, penton base-encoding sequence, VA RNA-encoding sequence, pIIIa protein-encoding sequence, or other early or late gene product-encoding sequences. Such deletions can be any length that results in the deletion of one or more encoded amino acids and in a reduction or elimination of the normal function of that polypeptide. For example, portions of a nucleic acid sequence of an adenovirus can be removed such that the otherwise encoded polypeptide lacks 5, 6, 7, 8, 9, 10, 15, 20, 25, 30, or more amino acid residues and lacks its normal activity. The portion or portions to be deleted can be removed from any location along the length of the sequence. For example, a portion of an adenovirus nucleic acid sequence can be removed at the 5′ end, the 3′ end, or an internal region of an adenovirus nucleic acid such as a fiber protein-encoding sequence, V protein-encoding sequence, hexon-encoding sequence, penton base-encoding sequence, VA RNA-encoding sequence, pIIIa protein-encoding sequence, or other early or late gene product-encoding sequences. In some cases, a SC-Ad can be as described elsewhere (see, e.g., Matchett et al., J. Virol., 93(10):e02016-18 (2019); International PCT Patent Application Publication No. WO 2009/111738; or International PCT Patent Application Publication No. WO 2022/040204).

An adenovirus vector provided herein (e.g., an adenovirus vector encoding one or more immunogens) can include a nucleic acid sequence encoding any appropriate immunogen (e.g., a nucleic acid that drives expression of any appropriate immunogen). In some cases, an immunogen can be an antigen. An immunogen can be a full-length immunogenic polypeptide or a portion thereof (e.g., can be derived from an immunogenic polypeptide).

When an immunogenic polypeptide is from a pathogen, the immunogenic polypeptide can be from any type of pathogen (e.g., a virus, a bacterium, a protozoan, a prion, a viroid, or a fungus). In some cases, an immunogenic polypeptide can be a polypeptide expressed by a virus (e.g., a viral polypeptide). For example, an immunogenic polypeptide can be a polypeptide expressed by a coronavirus (e.g., a beta-coronavirus). Examples of viruses that can express an immunogenic polypeptide include, without limitation, SARS-CoV, HCoV NL63, HKU1, MERS-COV, SARS-CoV-2, HIV-1, hepatitis B virus, hepatitis C virus, hepatitis D virus, hepatitis E virus, influenza, Ebola virus, Chiningunya virus, Zika virus, cytomegalovirus, West Nile virus, respiratory syncytial virus (RSV), metapneumovirus (MPV), a rhinovirus, a bocavirus, parainfluenza virus (PIV), and those described in Table 3-1 of “Learning from SARS: Preparing for the Next Disease Outbreak: Workshop Summary.” Institute of Medicine (US) Forum on Microbial Threats; Knobler S, Mahmoud A, Lemon S, et al., editors. Washington (DC): National Academies Press (US); 2004. In some cases, an immunogen can be derived from a polypeptide expressed by a bacterium (e.g., a bacterial polypeptide). Examples of bacteria that express polypeptides from which an immunogen can be derived include, without limitation, Clostridium (e.g., C. difficile), Staphylococcus aureus (e.g. methicillin-resistant S. aureus), Campylobacter (e.g. Campylobacter jejuni), Mycobacteria (e.g. M. tuberculosis), and Borrelia (B. burgdorferi). Examples of immunogenic polypeptides that can be expressed by a pathogen include, without limitation, C. difficile Toxin A (TcdA) polypeptides, C. difficile Toxin B (TcdB) polypeptides, coronavirus Spike polypeptides, the amino acid sequence set forth in SEQ ID NO:1 of International PCT Patent Publication No. WO 2022/040204, coronavirus nucleoproteins, coronavirus membrane proteins, coronavirus envelope proteins, coronavirus non-structural proteins (e.g., coronavirus non-structural proteins 1-16), influenza haemagglutinin polypeptides, RSV fusion (F) protein polypeptides, MPV F protein polypeptides, rhinovirus VP1 protein polypeptides, and PIV F protein polypeptides. For example, an immunogenic polypeptide associated with a pathogen can have, or can be encoded by, a sequence set forth in, for example, National Center for Biotechnology Information (NCBI) Accession Nos: MN938384 and AY772062.

When an immunogenic polypeptide is from an allergen, the immunogenic polypeptide can be from any type of allergen (e.g., a substance capable of triggering an immune response that results in an allergic reaction). Examples of allergens that can express and/or shed an immunogenic polypeptide include, without limitation, Fel d 7, Can fl, beta-lactoglobulin, prolamin, parvalbumin, gliadin, Fel d1, chitinase, glutenin, cupin, prolamin, profilins, polcalcins, bet v-1-related proteins, 2S albumins, vicilins, legumins, nsLTPs, and Aed a 2. For example, adenovirus vectors encoding one or more immunogens described herein can be used to deliver intranasally immunogens to a mammal (e.g., a human) such that the mammal produces antibodies (e.g., IgG and IgA antibodies) against an allergen associated with those immunogens. For example, nucleic acid molecules that can encode an adenovirus vector encoding one or more immunogens can be used to deliver intranasally immunogens to a mammal (e.g., a human) such that the mammal produces antibodies (e.g., IgG and IgA antibodies) against an allergen associated with those immunogens. For example, an immunogenic polypeptide associated with an allergen can have, or can be encoded by, a sequence set forth in, for example, NCBI Accession Nos: NP_001363134.1, AAD56719, NP_001363136.1, NP_001363139.1, P27762.1, P10414.2, P15494.2, P43176.2, NP_001191706.1, NP_001003190.1, XP_030099003.1, or XP 001657779.1.

When an immunogenic polypeptide is from a cancer cell (e.g., a cancer cell within a mammal having cancer), the immunogenic polypeptide can be expressed by any cancer cell. For example, an immunogenic polypeptide expressed by a cancer cell can be a tumor antigen. In some cases, an immunogenic polypeptide expressed by a cancer cell can be a cell surface tumor antigen. In some cases, an immunogenic polypeptide expressed by a cancer cell can be a tumor-associated antigen (TAA; e.g., an antigen, such as an abnormal protein, present on tumor cells). In some cases, an immunogenic polypeptide can be a tumor-specific antigen (TSA; e.g., an antigen present only on tumor cells). Examples of immunogenic polypeptides that can be expressed by a cancer cell and used as described herein include, without limitation, folate receptor alpha, mucin 1 (MUC-1), human epidermal growth factor receptor 2 (HER-2), estrogen receptor (ER), epidermal growth factor receptor (EGFR), folate receptor alpha, mesothelin, alphafetoprotein (AFP), carcinoembryonic antigen (CEA), CA-125, epithelial tumor antigen (ETA), melanoma-associated antigen (MAGE), antigens produced by Epstein-Barr Viruses, and antigens produced by human papilloma viruses. For example, adenovirus vectors encoding one or more immunogens as described herein can be used to deliver intranasally immunogens to a mammal (e.g., a human) such that the mammal produces antibodies (IgG and IgA antibodies) against cancer cells associated with those immunogens. For example, nucleic acid molecules that can encode an adenovirus vector encoding one or more immunogens as described herein can be used to deliver intranasally immunogens to a mammal (e.g., a human) such that the mammal produces antibodies (e.g., IgG and IgA antibodies) against cancer cells associated with those immunogens. For example, an immunogenic polypeptide associated with a cancer cell can have, or can be encoded by, a sequence set forth in, for example, NCBI Accession Nos: XP_002754883.1, AAA03229.1, Q02496.2, AAD33253.1, CEQ32409.1, YP_401631.1, YP_401632.1, or QAR15051.1.

When an immunogenic polypeptide is from a cancer cell (e.g., a cancer cell within a mammal having cancer), the immunogenic polypeptide can be expressed by any type of cancer cell. Examples of such cancers include, without limitation, lung cancers, breast cancers, prostate cancers, liver cancer, kidney cancers, brain cancers, B cell cancers, T cell cancers, ovarian cancers, and skin cancers.

An immunogen can be a full-length immunogenic polypeptide or a portion thereof (e.g., can be derived from an immunogenic polypeptide). For example, a nucleic acid sequence encoding an immunogenic polypeptide can be modified to remove portions of nucleic acid such that the encoded polypeptide lacks any number of amino acids (e.g., 5, 10, 15, 20, 30 amino acids, or all amino acids of the immunogenic polypeptide). In some cases, portions of a nucleic acid sequence encoding an immunogenic polypeptide can be removed from anywhere along the length of the sequence. For example, portions of the nucleic acid sequence can be removed at the 5′ end, the 3′ end, or an internal region of the target nucleic acid. In some cases, an immunogen can be designed to be secreted from cells infected with the adenovirus vector encoding the immunogen. For example, a nucleic acid sequence encoding an ER retention sequence can be removed from a nucleic acid sequence encoding an immunogen (e.g., such that the encoded immunogen lacks an ER retention sequence). In some cases, an immunogen can be designed to extend from cells infected with the adenovirus vector encoding the immunogen into the extracellular space. For example, an immunogen can include an ectodomain of an immunogenic polypeptide. In some cases, an immunogen can bind (e.g., can be designed to bind) to viral receptor (e.g., an ACE2 polypeptide). For example, an immunogen can include a receptor binding domain of an immunogenic polypeptide.

In some cases, an immunogen can include (e.g., can be a fusion polypeptide of) two or more immunogenic polypeptides described herein. For example, an immunogen can include a first immunogenic polypeptide and a second immunogenic polypeptide. In some cases, a first immunogenic polypeptide and a second immunogenic polypeptide can be different polypeptides. For example, an immunogen can include a tcdA polypeptide and a tcdB polypeptide (e.g., a tcdA/B fusion polypeptide). In some cases, a first immunogenic polypeptide and a second immunogenic polypeptide can be the same polypeptide. For example, an immunogen can include a tcdB polypeptide and a tcdB polypeptide (e.g., a tcdB/B fusion polypeptide). When an immunogen includes a first immunogenic polypeptide and a second immunogenic polypeptide, the first immunogenic polypeptide can be derived from a first pathogen, and the second immunogenic polypeptide can be derived from a second pathogen. The first pathogen and the second pathogen can be the same pathogen or different pathogens. When the first pathogen and the second pathogen are the same pathogen, the first immunogenic polypeptide and the second immunogenic polypeptide can be derived from the different strains of that pathogen. For example, a first immunogenic polypeptide and a second immunogenic polypeptide can be derived from different strains of the same bacterium (e.g., C. difficile).

Examples of immunogens derived from immunogenic polypeptides that can be used as described herein include, without limitation, the amino acid sequence set forth in SEQ ID NO: 2 of International PCT Patent Publication No. WO 2022/040204, the amino acid sequence set forth in SEQ ID NO:3 of International PCT Patent Publication No. WO 2022/040204, the amino acid sequence set forth in SEQ ID NO:4 of International PCT Patent Publication No. WO 2022/040204, the amino acid sequence set forth in SEQ ID NO:11 of International PCT Patent Publication No. WO 2022/040204, the amino acid sequence set forth in SEQ ID NO: 12 of International PCT Patent Publication No. WO 2022/040204, the amino acid sequence set forth in SEQ ID NO:13 of International PCT Patent Publication No. WO 2022/040204, the amino acid sequence set forth in SEQ ID NO: 14 of International PCT Patent Publication No. WO 2022/040204, the amino acid sequence set forth in SEQ ID NO: 15 of International PCT Patent Publication No. WO 2022/040204, the amino acid sequence set forth in SEQ ID NO: 16 of International PCT Patent Publication No. WO 2022/040204, the amino acid sequence set forth in SEQ ID NO:17 of International PCT Patent Publication No. WO 2022/040204, the amino acid sequence set forth in SEQ ID NO: 18 of International PCT Patent Publication No. WO 2022/040204, the amino acid sequence set forth in SEQ ID NO: 19 of International PCT Patent Publication No. WO 2022/040204, the amino acid sequence set forth in SEQ ID NO:42 of International PCT Patent Publication No. WO 2022/040204, the amino acid sequence set forth in SEQ ID NO: 43 of International PCT Patent Publication No. WO 2022/040204, the amino acid sequence set forth in SEQ ID NO:44 of International PCT Patent Publication No. WO 2022/040204, the amino acid sequence set forth in SEQ ID NO:45 of International PCT Patent Publication No. WO 2022/040204, the amino acid sequence set forth in SEQ ID NO: 46 of International PCT Patent Publication No. WO 2022/040204, the amino acid sequence set forth in SEQ ID NO:47 of International PCT Patent Publication No. WO 2022/040204, and the amino acid sequence set forth in SEQ ID NO:48 of International PCT Patent Publication No. WO 2022/040204.

In some cases, an immunogen described herein can be a variant of a wild-type immunogen. For example, a variant of a coronavirus Spike polypeptide (e.g., a SARS-CoV-2 Spike polypeptide) can comprise or consist essentially of an amino acid sequence set forth in any one of SEQ ID NOs:1-4 of International PCT Patent Publication No. WO 2022/040204 with one or more (e.g., one, two, three, four, five, six, seven, eight, nine, ten, or more) amino acid deletions, additions, substitutions, or combinations thereof. Examples of amino acid deletions that can be present in a variant of a coronavirus Spike polypeptide (e.g., as numbered in any one of SEQ ID NOs:1-4 of International PCT Patent Publication No. WO 2022/040204) include, without limitation, a deletion of residues 69-70, a deletion of residue 144, a deletion of residues 156-157, a deletion of residues 241-243, and a deletion of residues 246-252. Examples of amino acid substitutions that can be present in a variant of a coronavirus Spike polypeptide (e.g., as numbered in any one of SEQ ID NOs:1-4 of International PCT Patent Publication No. WO 2022/040204), include, without limitation, a L5F amino acid substitution, a S13I amino acid substitution, a L18F amino acid substitution, a T19R amino acid substitution, a T20N amino acid substitution, a P26S amino acid substitution, a G75I amino acid substitution, a A67V amino acid substitution, a V70F amino acid substitution, a T76I amino acid substitution, a D80A amino acid substitution, a D80G amino acid substitution, a T95I amino acid substitution, a D138Y amino acid substitution, a G142D amino acid substitution, a W152C amino acid substitution, a E154K amino acid substitution, a F157S amino acid substitution, a R158G amino acid substitution, a R190S amino acid substitution, a D215G amino acid substitution, a A222V amino acid substitution, a D253G amino acid substitution, a W258L amino acid substitution, a K417N amino acid substitution, a K417T amino acid substitution, a L452R amino acid substitution, a L452Q amino acid substitution, a Y453F amino acid substitution, a S477N amino acid substitution, a T478K amino acid substitution, a E484Q amino acid substitution, a E484K amino acid substitution, a F490S amino acid substitution, a E484K amino acid substitution, a S494P amino acid substitution, a N501Y amino acid substitution, a A570D amino acid substitution, a D614G amino acid substitution, a H655Y amino acid substitution, a Q677H amino acid substitution, a P681H amino acid substitution, a P681R amino acid substitution, a A701V amino acid substitution, a T716I amino acid substitution, a T859N amino acid substitution, a F888L amino substitution, a D950N amino acid substitution, a Q957R amino acid substitution, a S982A amino acid substitution, a K986P amino acid substitution, a V987P amino acid substitution, a T1027I amino acid substitution, a Q1071H amino acid substitution, a D1118H amino acid substitution, and a K119IN amino acid substitution. For example, a variant of a coronavirus Spike polypeptide can include a K986P amino acid substitution and a V987P amino acid substitution (e.g., a PP substitution). In some cases, a variant of a coronavirus Spike polypeptide can be a gamma mink variant of a coronavirus Spike polypeptide. In some cases, a variant of a coronavirus Spike polypeptide can be a delta variant of a coronavirus Spike polypeptide. In some cases, a variant of a coronavirus Spike polypeptide can be a lambda variant of a coronavirus Spike polypeptide. In some cases, a variant of a coronavirus Spike polypeptide can be an epsilon variant of a coronavirus Spike polypeptide. In some cases, a variant of a coronavirus Spike polypeptide can be a delta plus variant of a coronavirus Spike polypeptide.

In some cases, an immunogen described herein can have an amino acid sequence with at least 85% sequence identity (e.g., at least 88% sequence identity, at least 90% sequence identity, at least 93% sequence identity, at least 95% sequence identity, at least 97% sequence identity, at least 98% sequence identity, or at least 99% sequence identity) to the amino acid sequence set forth in any one of SEQ ID NOs:1-4 of International PCT Patent Publication No. WO 2022/040204.

The percent sequence identity between a particular amino acid sequence and a sequence referenced by a particular sequence identification number is determined as follows. First, an amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1-r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. A matched position refers to a position in which identical amino acid occur at the same position in aligned sequences. The percent sequence identity is determined by dividing the number of matches by the length of the sequence set forth in the identified sequence (e.g., SEQ ID NO: 1 of International PCT Patent Publication No. WO 2022/040204, SEQ ID NO:2 of International PCT Patent Publication No. WO 2022/040204, SEQ ID NO:3 of International PCT Patent Publication No. WO 2022/040204, or SEQ ID NO:4 of International PCT Patent Publication No. WO 2022/040204), followed by multiplying the resulting value by 100. For example, an amino acid sequence that has 220 matches when aligned with the sequence set forth in SEQ ID NO:2 of International PCT Patent Publication No. WO 2022/040204 is 93.2 percent identical to the sequence set forth in SEQ ID NO:2 of International PCT Patent Publication No. WO 2022/040204 (i.e., 220÷236×100=93.2). It is noted that the percent sequence identity value is rounded to the nearest tenth. For example, 75.1, 75.2, 75.3, and 75.4 is rounded down to 75, while 75.5, 75.6, 75.7, 75.8, and 75.9 is rounded up to 76. It also is noted that the length value will always be an integer.

In some cases, a coronavirus Spike polypeptide variant can contain the entire amino acid sequence set forth in any one of SEQ ID NOs:1-4 of International PCT Patent Publication No. WO 2022/040204, except that the amino acid sequence contains from one to ten (e.g., one to nine, two to nine, one to eight, two to eight, one to seven, one to six, one to five, one to four, one to three, two, or one) amino acid additions, deletions, substitutions, or combinations thereof, provided that the coronavirus Spike polypeptide variant has the ability to induce an immune response against a coronavirus within a mammal (e.g., a human). In some cases, a coronavirus Spike polypeptide variant can consist essentially of the amino acid sequence set forth in any one of SEQ ID NOs:1-4 of International PCT Patent Publication No. WO 2022/040204 except that the amino acid sequence contains one, two, three, four, or five amino acid residues preceding the articulated sequence of the sequence identifier (e.g., SEQ ID NO:1 of International PCT Patent Publication No. WO 2022/040204), and/or has one, two, three, four, or five amino acid residues following the articulated sequence of the sequence identifier (e.g., SEQ ID NO:1 of International PCT Patent Publication No. WO 2022/040204), provided that the coronavirus Spike polypeptide has the ability to induce an immune response against a coronavirus within a mammal (e.g., a human).

In some cases, an adenovirus vector provided herein (e.g., an adenovirus vector encoding one or more immunogens) can include nucleic acid sequence encoding two or more (e.g., two, three, four, five, six, seven, eight, nine, ten, or more) immunogens from different immunogenic polypeptides. For example, an adenovirus vector provided herein can include nucleic acid sequence encoding an immunogen derived from a first pathogen (e.g., an immunogen derived from an immunogenic polypeptide expressed by SARS-CoV-2) and can include nucleic acid sequence encoding an immunogen derived from a second pathogen (e.g., an immunogen derived from an immunogenic polypeptide expressed by a pathogen other than SARS-CoV-2). In some cases, an adenovirus vector provided herein that includes a nucleic acid sequence encoding two or more immunogens derived from an immunogenic polypeptide expressed by different pathogens can include nucleic acid sequence encoding a polypeptide comprising, consisting of, or consisting essentially of the amino acid sequence set forth in any one of SEQ ID NOs:1-4 of International PCT Patent Publication No. WO 2022/040204 and can include nucleic acid sequence encoding a polypeptide comprising, consisting of, or consisting essentially of the amino acid sequence set forth in any one of SEQ ID NOs: 20-21 of International PCT Patent Publication No. WO 2022/040204. When an adenovirus vector provided herein includes nucleic acid sequence encoding two or more immunogens from immunogenic polypeptides expressed by different pathogens, the adenovirus vector can be used to induce an immune response against two or more pathogens.

In some cases, an adenovirus vector provided herein (e.g., an adenovirus vector encoding one or more immunogens) can include nucleic acid sequence encoding two or more (e.g., two, three, four, five, six, seven, eight, nine, ten, or more) immunogens from the same immunogenic polypeptide and/or the same pathogen. For example, an adenovirus vector provided herein can include nucleic acid sequence encoding two or more immunogens derived from the same pathogen. In some cases, an adenovirus vector provided herein that includes a nucleic acid sequence encoding two or more immunogens derived from an immunogenic polypeptide expressed by influenza can include nucleic acid sequence encoding a polypeptide comprising, consisting of, or consisting essentially of the amino acid sequence set forth in SEQ ID NO:20 of International PCT Patent Publication No. WO 2022/040204 and can include nucleic acid sequence encoding a polypeptide comprising, consisting of, or consisting essentially of the amino acid sequence set forth in SEQ ID NO:21 of International PCT Patent Publication No. WO 2022/040204.

In some cases, an adenovirus vector provided herein (e.g., an adenovirus vector encoding one or more immunogens) that includes a nucleic acid sequence encoding one or more (e.g., one, two, three, four, five, six, seven, eight, nine, ten, or more) immunogens also can include one or more regulatory sequences (e.g., an enhancer or a promoter sequence such as a constitutive, inducible, and/or tissue-specific promoter sequence) to drive transcription of the immunogen(s). Examples of enhancers and promoters that can be used to drive expression of a nucleic acid sequence encoding one or more immunogens of an adenovirus provided herein include, without limitation, a CMV enhancer sequence, a CMV promoter sequence, a CAG enhancer sequence, a CAG promoter sequence, a RSV enhancer sequence, a RSV promoter sequence, a Ef1alpha enhancer sequence, a Ef1alpha promoter sequence, a ubiquitin enhancer sequence, a ubiquitin promoter sequence, adenovirus enhancer sequences, and adenovirus promoter sequences. Any appropriate method can be used to detect expression of an immunogen from adenovirus vector infected cells. For example, antibodies that recognize an immunogen can be used to detect the presence or absence of the immunogen.

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) adjuvant polypeptides (e.g., nucleic acid that drives expression of one or more adjuvant polypeptides). For example, an adenovirus vector can include a nucleic acid sequence encoding one or more polypeptides that can enhance an immune response within a mammal. In some cases, an adjuvant polypeptide can be a cytokine. In some cases, an adjuvant polypeptide can be an immune stimulator. In some cases, an adjuvant polypeptide can be a toxin. In some cases, an adjuvant polypeptide can accelerate a systemic T cell response against a pathogen present within a mammal. In some cases, an adjuvant polypeptide can increase a concentration of antibodies against a pathogen at a site where the pathogen can enter a mammal's body. For example, an adjuvant polypeptide can increase a concentration of antibodies against a virus (e.g., a coronavirus) at a mucosal site where the virus can enter a mammal's body. Examples of adjuvant polypeptides that can be encoded by an adenovirus vectors encoding one or more immunogens described herein include, without limitation, granulocyte-macrophage colony-stimulating factor (GM-CSF) polypeptides, interleukin 4 (IL-4) polypeptides, interleukin 21 (IL-21) polypeptides, CD40 ligand (CD40L) polypeptides, 4-1BB ligand (4-1BBL) polypeptides, transforming growth factor beta (TGF-β) polypeptides, C. difficile toxin polypeptides (e.g., C. difficile TcdA polypeptides (see, e.g., SEQ ID NO:22 of International PCT Patent Publication No. WO 2022/040204), C. difficile TcdB polypeptides (see, e.g., SEQ ID NO: 23 of International PCT Patent Publication No. WO 2022/040204), and/or the amino acid sequence set forth in SEQ ID NO: 10 of International PCT Patent Publication No. WO 2022/040204), and influenza polypeptides (e.g., N polypeptides, H polypeptides, M polypeptides, the amino acid sequence set forth in SEQ ID NO:20 of International PCT Patent Publication No. WO 2022/040204, and/or the amino acid sequence set forth in SEQ ID NO: 21 of International PCT Patent Publication No. WO 2022/040204). In some cases, an adjuvant polypeptide (e.g., SEQ ID NO:22 of International PCT Patent Publication No. WO 2022/040204) can be preceded by an AAT secretory sequence (e.g., SEQ ID NO:28 of International PCT Patent Publication No. WO 2022/040204). In some cases, a nucleic acid sequence encoding an adjuvant polypeptide (e.g., SEQ ID NO:23 of International PCT Patent Publication No. WO 2022/040204) can be preceded by a cleavage site such as a synthetic furin cleavage site (e.g., SEQ ID NO:29 of International PCT Patent Publication No. WO 2022/040204). Examples of nucleic acid sequences that can encoding an adjuvant polypeptide described herein include, without limitation, the nucleic acid sequence set forth in SEQ ID NO:24 of International PCT Patent Publication No. WO 2022/040204 and the nucleic acid sequence set forth in SEQ ID NO:25 of International PCT Patent Publication No. WO 2022/040204. In some cases, a nucleic acid sequence encoding an adjuvant polypeptide (e.g., SEQ ID NO:24 of International PCT Patent Publication No. WO 2022/040204) can be preceded by an AAT secretory sequence (e.g., SEQ ID NO:40 of International PCT Patent Publication No. WO 2022/040204). In some cases, a nucleic acid sequence encoding an adjuvant polypeptide (e.g., SEQ ID NO:25 of International PCT Patent Publication No. WO 2022/040204) can be preceded by a cleavage site such as a synthetic furin cleavage site (e.g., SEQ ID NO:41 of International PCT Patent Publication No. WO 2022/040204). An adjuvant polypeptide can be a full-length polypeptide or a fragment of an adjuvant polypeptide described herein provided that the fragment has the ability to enhance an immune response (e.g., a biologically active fragment). In some cases, an adjuvant polypeptide can be as described elsewhere (see, e.g., Matchett et al., Vaccines, 8(1):64 (2020)).

In some cases, an adenovirus vector provided herein (e.g., an adenovirus vector encoding one or more immunogens) can encode (e.g., can be designed to encode) a polypeptide that includes an immunogen fused to an adjuvant polypeptide. For example, a nucleic acid sequence encoding an immunogen can be fused to a nucleic acid sequence encoding an adjuvant polypeptide (e.g., such that the encoded immunogen is fused to the encoded adjuvant polypeptide). An example of an immunogen fused to an adjuvant polypeptide that can be encoded by an adenovirus vectors encoding one or more immunogens described herein include, without limitation, the amino acid sequence set forth in SEQ ID NO:5 of International PCT Patent Publication No. WO 2022/040204.

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) chaff polypeptides (e.g., nucleic acid that drives expression of one or more chaff polypeptides). A chaff polypeptide can be a full-length polypeptide or a fragment thereof provided that it reduces the rate of entry or inhibits entry of a pathogen into a cell within a mammal. In some cases, a chaff polypeptide can be a soluble polypeptide. For example, a soluble chaff polypeptide can be a full-length chaff polypeptide or a fragment of a chaff polypeptide that lacks a transmembrane domain. For example, a soluble chaff polypeptide can include an ectodomain of a chaff polypeptide. In some cases, a chaff polypeptide can target (e.g., target and bind to) a particular pathogen (e.g., a virus such as a coronavirus) to reduce the rate of entry or inhibit entry of the pathogen into a cell within a mammal. In some cases, a chaff polypeptide can target (e.g., target and bind to) two, three, four, five, six, or more different pathogens. In some cases, a chaff polypeptide can include one or more mutations (e.g., inactivating mutations). Examples of chaff polypeptides that can be encoded by an adenovirus vectors encoding one or more immunogens described herein include, without limitation, full-length ACE2 polypeptides and fragments thereof, full-length CD13 polypeptides and fragments thereof, full-length CEACAM1 polypeptides and fragments thereof, full-length sialylated polypeptides and fragments thereof, full-length CD46 polypeptides and fragments thereof, full-length nestin polypeptides and fragments thereof, the amino acid sequence set forth in SEQ ID NO:8 of International PCT Patent Publication No. WO 2022/040204, and the amino acid sequence set forth in SEQ ID NO:9 of International PCT Patent Publication No. WO 2022/040204.

In some cases, an adenovirus vector provided herein (e.g., an adenovirus vector encoding one or more immunogens) can encode (e.g., can be designed to encode) a polypeptide that includes an immunogen fused to a chaff polypeptide. For example, a nucleic acid sequence encoding an immunogen can include a nucleic acid sequence encoding a chaff polypeptide (e.g., such that the encoded immunogen is fused to the encoded chaff polypeptide).

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) marker polypeptides (e.g., nucleic acid that drives expression of one or more marker polypeptides). Examples of marker polypeptides that can be encoded by an adenovirus vector encoding one or more immunogens described herein include, without limitation, fluorescent polypeptides (e.g., GFP, RFP, CFP, and YFP), streptavidin polypeptides, Cre recombinase polypeptides, Cas polypeptides, luciferase polypeptides, betagalactosidase polypeptides, and sodium iodide symporter polypeptides.

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) polypeptides that can form a multimer (e.g., nucleic acid that drives expression of one or more polypeptides that can form a multimer). Examples of polypeptides that can form a multimer that can be encoded by an adenovirus vectors encoding one or more immunogens described herein include, without limitation, immunoglobulin constant region polypeptides (e.g., an Ig polypeptide), streptavidin polypeptides, and sigma coil polypeptides.

In some cases, an adenovirus vector provided herein (e.g., an adenovirus vector encoding one or more immunogens) can encode (e.g., can be designed to encode) a polypeptide that includes an immunogen fused to a polypeptide that can form a multimer. For example, a nucleic acid sequence encoding an immunogen can include a nucleic acid sequence encoding a polypeptide that can form a multimer (e.g., such that the encoded immunogen is fused to the encoded polypeptide that can form a multimer). Examples of immunogens fused to a polypeptide that can form a multimer that can be encoded by an adenovirus vectors encoding one or more immunogens described herein include, without limitation, the amino acid sequence set forth in SEQ ID NO:5 of International PCT Patent Publication No. WO 2022/040204, the amino acid sequence set forth in SEQ ID NO:6 of International PCT Patent Publication No. WO 2022/040204, and the amino acid sequence set forth in SEQ ID NO:7 of International PCT Patent Publication No. WO 2022/040204.

In some cases, an adenovirus vector provided herein (e.g., an adenovirus vector encoding one or more immunogens) can have a genome that is at least 85% percent identical (e.g., at least 88% sequence identical, at least 90% sequence identical, at least 93% sequence identical, at least 95% sequence identical, at least 97% sequence identical, at least 98% sequence identical, or at least 99% sequence identical) to a sequence set forth in any one of SEQ ID NOs: 28-39 of International PCT Patent Publication No. WO 2022/040204. For example, an adenovirus vector provided herein can have a genome that comprising, consisting of, or consisting essentially of the nucleic acid sequence set forth in SEQ ID NO: 28 of International PCT Patent Publication No. WO 2022/040204. For example, an adenovirus vector provided herein can have a genome that comprising, consisting of, or consisting essentially of the nucleic acid sequence set forth in SEQ ID NO:29 of International PCT Patent Publication No. WO 2022/040204. For example, an adenovirus vector provided herein can have a genome that comprising, consisting of, or consisting essentially of the nucleic acid sequence set forth in SEQ ID NO:30 of International PCT Patent Publication No. WO 2022/040204. For example, an adenovirus vector provided herein can have a genome that comprising, consisting of, or consisting essentially of the nucleic acid sequence set forth in SEQ ID NO:31 of International PCT Patent Publication No. WO 2022/040204. For example, an adenovirus vector provided herein can have a genome that comprising, consisting of, or consisting essentially of the nucleic acid sequence set forth in SEQ ID NO: 32 of International PCT Patent Publication No. WO 2022/040204. For example, an adenovirus vector provided herein can have a genome that comprising, consisting of, or consisting essentially of the nucleic acid sequence set forth in SEQ ID NO:33 of International PCT Patent Publication No. WO 2022/040204. For example, an adenovirus vector provided herein can have a genome that comprising, consisting of, or consisting essentially of the nucleic acid sequence set forth in SEQ ID NO:34 of International PCT Patent Publication No. WO 2022/040204. For example, an adenovirus vector provided herein can have a genome that comprising, consisting of, or consisting essentially of the nucleic acid sequence set forth in SEQ ID NO:35 of International PCT Patent Publication No. WO 2022/040204. For example, an adenovirus vector provided herein can have a genome that comprising, consisting of, or consisting essentially of the nucleic acid sequence set forth in SEQ ID NO: 36 of International PCT Patent Publication No. WO 2022/040204. For example, an adenovirus vector provided herein can have a genome that comprising, consisting of, or consisting essentially of the nucleic acid sequence set forth in SEQ ID NO:37 of International PCT Patent Publication No. WO 2022/040204. For example, an adenovirus vector provided herein can have a genome that comprising, consisting of, or consisting essentially of the nucleic acid sequence set forth in SEQ ID NO:38 of International PCT Patent Publication No. WO 2022/040204. For example, an adenovirus vector provided herein can have a genome that comprising, consisting of, or consisting essentially of the nucleic acid sequence set forth in SEQ ID NO:39 of International PCT Patent Publication No. WO 2022/040204.

In some cases, an adenovirus vector provided herein (e.g., an adenovirus vector encoding one or more immunogens) can have a genome that includes one or more of the coding regions set forth in any one of SEQ ID NOs: 28-39 of International PCT Patent Publication No. WO 2022/040204. For example, a SC-Ad can be designed to have a genome where all the encoded polypeptides of the SC-Ad have the same amino acid sequence as those polypeptides encoded by the nucleic acid set forth in any one of SEQ ID NOs: 28-39 of International PCT Patent Publication No. WO 2022/040204.

In some cases, nucleic acid molecules can be designed to encode an adenovirus vector provided herein (e.g., an adenovirus vector encoding one or more immunogens such as a SC-Ad described herein). The term “nucleic acid” as used herein encompasses both RNA and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

In some cases, cells (e.g., cell lines) can be designed to contain one or more adenovirus vectors described herein (e.g., one or more adenovirus vectors encoding one or more immunogens such as SC-Ads described herein). In cases where an adenovirus vector lacks all or a portion of at least of one adenovirus sequences, the cells containing the adenovirus vector can provide the missing adenovirus polypeptide. For example, when the adenovirus is designed to lack nucleic acid encoding the adenovirus fiber polypeptide, an adenovirus fiber polypeptide-expressing cell line can be used to generate the adenovirus such that the adenovirus contains the fiber polypeptide (e.g., the wild-type fiber polypeptide) while lacking the nucleic acid that encodes the fiber polypeptide (e.g., the wild-type fiber polypeptide). For example, when the adenovirus is designed to lack nucleic acid encoding the V polypeptide, an adenovirus V polypeptide-expressing cell line can be used to generate the adenovirus such that the adenovirus contains the V polypeptide (e.g., the wild-type V polypeptide) while lacking the nucleic acid that encodes the V polypeptide (e.g., the wild-type V polypeptide). For example, when the adenovirus is designed to lack nucleic acid encoding the pIIIa polypeptide, an adenovirus pIIIa polypeptide-expressing cell line can be used to generate the adenovirus such that the adenovirus contains the pIIIa polypeptide (e.g., the wild-type pIIIa polypeptide) while lacking the nucleic acid that encodes the pIIIa polypeptide (e.g., the wild-type pIIIa polypeptide). In some cases, cells containing adenovirus vectors described herein can increase the available number of copies of that virus by at least 100-fold (e.g., by 100-fold to 15,000-fold, by 500- to 10,000-fold, by 5,000- to 10,000-fold, or by 5,000- to 15,000-fold). A virus can be expanded until a desired concentration is obtained in standard cell culture media (e.g., DMEM or RPMI-1640 supplemented with 5-10% fetal bovine serum at 37 ° C in 5% CO₂). A viral titer typically is assayed by inoculating cells (e.g., A549 or 293 cells) in culture or by quantitating viral genomes by optical density or real-time PCR. In some cases, cells containing an adenovirus vector provided herein can be used to propagate the adenovirus vector (e.g., to establish a stock of the adenovirus vector). For example, a stock of the adenovirus vector can be produced by growth in mammalian cells. In some cases, a stock of the adenovirus vector can be aliquoted and frozen, and can be stored at −70 ° C to −80 ° C (e.g., at concentrations higher than the therapeutically effective dose). In some cases, a stock of the adenovirus vector can be stored in a stabilizing solution. Examples of stabilizing solutions include, without limitation, sugars (e.g., trehalose, dextrose, and glucose), amino acids, glycerol, gelatin, monosodium glutamate, Ca²⁺, and Mg²⁺.

In some cases, adenovirus vectors described herein encoding one or more immunogens (and/or nucleic acid molecules that can encode an adenovirus vector described herein encoding one or more immunogens) can be formulated into a composition (e.g., a pharmaceutical composition such as a vaccine composition) for nasal administration to a mammal (e.g., a human). For example, adenovirus vectors described herein encoding one or more immunogens (and/or nucleic acid molecules that can encode an adenovirus vector described herein encoding one or more immunogens) can be formulated together with one or more pharmaceutically acceptable carriers (additives), excipients, and/or diluents for nasal administration. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil. Suitable pharmaceutical formulations depend in part upon the use and the route of administration. Such forms should not prevent the composition or formulation from reaching target cells or from exerting its effect.

In some cases, a composition including adenovirus vectors described herein encoding one or more immunogens (and/or nucleic acid molecules that can encode an adenovirus vector described herein encoding one or more immunogens) can include a plurality of identical adenovirus vectors that are designed to encode one or more immunogens (and/or a plurality of identical nucleic acid molecules that can encode an adenovirus vector described herein encoding one or more immunogens).

In some cases, a composition including adenovirus vectors described herein encoding one or more immunogens (and/or nucleic acid molecules that can encode an adenovirus vector described herein encoding one or more immunogens) can include a population of two or more (e.g., two, three, four, five, or more) different adenovirus vectors. For example, a composition can be designed to include two populations of adenovirus vectors, with the first population containing one or more coronavirus immunogens (e.g., an amino acid sequence set forth in any one of SEQ ID NOs:1-4, 42-44, 47, and 48 of International PCT Patent Publication No. WO 2022/040204) and the second population containing one or more adjuvant polypeptides (e.g., a GM-CSF polypeptide, an IL-4 polypeptide, an IL-21 polypeptide, a CD40L polypeptide, a 4-1BBL polypeptide, a TGF-β polypeptide, a C. difficile TcdA polypeptide, a C. difficile TcdB polypeptide, and/or biologically active fragments thereof). For example, a composition can be designed to include two populations of adenovirus vectors, with the first population containing one or more coronavirus immunogens (e.g., an amino acid sequence set forth in any one of SEQ ID NOs:1-4, 42-44, 47, and 48 of International PCT Patent Publication No. WO 2022/040204) and the second population containing a chaff polypeptide (e.g., a fragment of an ACE2 polypeptide).

In some cases, a composition including adenovirus vectors described herein encoding one or more immunogens (and/or nucleic acid molecules that can encode an adenovirus vector described herein encoding one or more immunogens) can be designed to include populations of different adenovirus vectors where each population of adenovirus vectors of the composition contains a single immunogen (and/or nucleic acid molecules that can encode an adenovirus vector described herein encoding a single immunogen). For example, a composition of adenovirus vectors can be designed to include a first population of adenovirus vectors having a first coronavirus immunogen (e.g., an amino acid sequence set forth in any one of SEQ ID NOs:1-4, 42-44, 47, and 48 of International PCT Patent Publication No. WO 2022/040204) and a second population of adenovirus vectors having a second coronavirus immunogen (e.g., an amino acid sequence set forth in any one of SEQ ID NOs:1-4, 42-44, 47, and 48 of International PCT Patent Publication No. WO 2022/040204). For example, a composition of adenovirus vectors can be designed to include a first population of adenovirus vectors having a first coronavirus immunogen (e.g., an amino acid sequence set forth in any one of SEQ ID NOs:1-4, 42-44, 47, and 48 of International PCT Patent Publication No. WO 2022/040204), a second population of adenovirus vectors having a second coronavirus immunogen (e.g., an amino acid sequence set forth in any one of SEQ ID NOs:1-4, 42-44, 47, and 48 of International PCT Patent Publication No. WO 2022/040204), and a third population of adenovirus vectors having a third coronavirus immunogen (e.g., an amino acid sequence set forth in any one of SEQ ID NOs:1-4, 42-44, 47, and 48 of International PCT Patent Publication No. WO 2022/040204). In another example, a composition of adenovirus vectors can be designed to include a first population of adenovirus vectors having a first coronavirus immunogen (e.g., an amino acid sequence set forth in any one of SEQ ID NOs: 1-4, 42-44, 47, and 48 of International PCT Patent Publication No. WO 2022/040204), a second population of adenovirus vectors having a second coronavirus immunogen (e.g., an amino acid sequence set forth in any one of SEQ ID NOs:1-4, 42-44, 47, and 48 of International PCT Patent Publication No. WO 2022/040204), and a third population of adenovirus vectors an adjuvant polypeptide (e.g., a GM-CSF polypeptide, an IL-4 polypeptide, an IL-21 polypeptide, a CD40L polypeptide, a 4-1BBL polypeptide, a TGF-β polypeptide, a C. difficile TcdA polypeptide, a C. difficile TcdB polypeptide, and/or biologically active fragments thereof). In another example, a composition of adenovirus vectors can be designed to include a first population of adenovirus vectors having a first coronavirus immunogen (e.g., an amino acid sequence set forth in any one of SEQ ID NOs: 1-4, 42-44, 47, and 48 of International PCT Patent Publication No. WO 2022/040204), a second population of adenovirus vectors having a second coronavirus immunogen (e.g., an amino acid sequence set forth in any one of SEQ ID NOs:1-4, 42-44, 47, and 48 of International PCT Patent Publication No. WO 2022/040204), and a third population of adenovirus vectors having a chaff polypeptide (e.g., a fragment of an ACE2 polypeptide).

This document also provides methods for using adenovirus vectors described herein (e.g., adenovirus vectors encoding one or more immunogens) and/or nucleic acid molecules that can encode an adenovirus vector described herein.

In some cases, adenovirus vectors described herein and/or nucleic acid molecules that can encode an adenovirus vector described herein can be administered intranasally to a mammal (e.g., a human) as a vaccination boost to increase an immune response (e.g., to increase IgG and IgA antibody responses and/or to increase T cell responses) against a pathogen (e.g., a bacterial or a viral pathogen associated with an immunogen encoded by the adenovirus vectors).

In some cases, adenovirus vectors described herein and/or nucleic acid molecules that can encode an adenovirus vector described herein can be administered intranasally to a mammal (e.g., a human) as a heterologous vaccination boost to increase an immune response (e.g., to increase IgG and IgA antibody responses and/or to increase T cell responses) against a pathogen (e.g., a bacterial or a viral pathogen associated with an immunogen encoded by the adenovirus vectors). For example, adenovirus vectors described herein encoding one or more immunogens (and/or nucleic acid molecules that can encode an adenovirus vector described herein encoding one or more immunogens) can be administered intranasally to a mammal (e.g., a human) as a heterologous vaccination boost to provide the mammal with an immune response (e.g., an IgG, IgA, and T cells immune response) effective to reduce the severity of an infection caused by a pathogen associated with the immunogen(s) encoded by the adenovirus vectors. In some cases, adenovirus vectors described herein encoding one or more immunogens (and/or nucleic acid molecules that can encode an adenovirus vector encoding one or more immunogens) can be administered intranasally to a mammal (e.g., a human) as described herein as a heterologous vaccination boost to provide the mammal with an immune response (e.g., an IgG, IgA, and T cells immune response) effective to prevent the mammal from exhibiting symptoms of an infection caused by a pathogen associated with the immunogen(s) encoded by the adenovirus vectors. In some cases, adenovirus vectors described herein and/or nucleic acid molecules that can encode an adenovirus vector described herein can be administered intranasally to a mammal (e.g., a human) as a heterologous vaccination boost to increase IgG, IgA, and T cell immune responses within the mammal.

In some cases, adenovirus vectors described herein and/or nucleic acid molecules that can encode an adenovirus vector described herein can be administered intranasally as a heterologous vaccination boost to a mammal (e.g., a human) that previously received a vaccine targeting the same pathogen (e.g., a bacterial or a viral pathogen) to increase the number of IgG and IgA antibodies (e.g., IgG and IgA antibodies against the pathogen associated with the immunogen(s) encoded by the adenovirus vectors) within the mammal. For example, adenovirus vectors described herein encoding one or more immunogens (and/or nucleic acid molecules that can encode an adenovirus vector described herein encoding one or more immunogens) can be administered intranasally as a heterologous vaccination boost to a mammal (e.g., a human) that previously received a vaccine targeting the same pathogen (e.g., a bacterial or a viral pathogen) to increase the number of IgG and IgA antibodies within the mammal by, for example, 10, 20, 30, 40, 50, 60, 70, 80, 90, 95, or more percent. In some cases, adenovirus vectors described herein and/or nucleic acid molecules that can encode an adenovirus vector described herein can be administered intranasally as a heterologous vaccination boost to a mammal (e.g., a human) that previously received a vaccine targeting the same pathogen (e.g., a bacterial or a viral pathogen) to produce IgG and IgA antibodies against the immunogen(s) within the mammal starting from about 15 days post administration (or starting from about 16 days post administration, or starting from about 17 days post administration, or starting from about 18 days post administration, or starting from about 19 days post administration, or starting from about 20 days post administration, or starting from about 21 days post administration, or starting from about 22 days post administration) and lasting at least to about 40 days post administration (e.g., lasting at least to about 43 days post administration, lasting at least to about 45 days post administration, lasting at least to about 50 days post administration, lasting at least to about 55 days post administration, lasting at least to about 60 days post administration, lasting at least to about 65 days post administration, lasting at least to about 70 days post administration, lasting at least to about 75 days post administration, lasting at least to about 80 days post administration, lasting at least to about 85 days post administration, lasting at least to about 90 days post administration, lasting at least to about 95 days post administration, lasting at least to about 100 days post administration, lasting at least to about 105 days post administration, lasting at least to about 110 days post administration, lasting at least to about 115 days post administration, lasting at least to about 120 days post administration, lasting at least to about 125 days post administration, lasting at least to about 130 days post administration, or lasting at least to about 135 days post administration). The previously received vaccine can be one that the mammal (e.g., the human) received from about 21 days to about 120 months (e.g., from about 21 days to about 60 months, from about 21 days to about 36 months, from about 21 days to about 24 months, from about 21 days to about 12 months, from about 21 days to about 8 months, from about 21 days to about 6 months, from about 21 days to about 4 months, from about 21 days to about 3 months, from about 21 days to about 2 months, from about 1 month to about 120 months, from about 3 months to about 120 months, from about 6 months to about 120 months, from about 12 months to about 120 months, from about 18 months to about 120 months, from about 24 months to about 120 months, from about 36 months to about 120 months, from about 48 months to about 120 months, from about 1 month to about 60 months, from about 2 months to about 48 months, from about 3 months to about 36 months, from about 4 months to about 24 months, from about 6 months to about 18 months, from about 1 month to about 3 months, from about 3 months to about 6 months, from about 6 months to about 12 months, from about 12 months to about 24 months, from about 24 months to about 48 months, from about 36 months to about 72 months, from about 48 months to about 84 months, or from about 60 months to about 96 months) before the mammal is intranasally administered an adenovirus vector described herein (and/or nucleic acid molecules that can encode an adenovirus vector described herein).

In some cases, adenovirus vectors described herein and/or nucleic acid molecules that can encode an adenovirus vector described herein can be administered intranasally as a vaccination boost to a mammal (e.g., a human) that previously received a vaccine targeting the same pathogen (e.g., a bacterial or a viral pathogen) to produce IgG and IgA antibodies against the immunogen(s) within the mammal starting from about 15 days post administration (or starting from about 16 days post administration, or starting from about 17 days post administration, or starting from about 18 days post administration, or starting from about 19 days post administration, or starting from about 20 days post administration, or starting from about 21 days post administration, or starting from about 22 days post administration) and lasting at least to about 40 days post administration (e.g., lasting at least to about 43 days post administration, lasting at least to about 45 days post administration, lasting at least to about 50 days post administration, lasting at least to about 55 days post administration, lasting at least to about 60 days post administration, lasting at least to about 65 days post administration, lasting at least to about 70 days post administration, lasting at least to about 75 days post administration, lasting at least to about 80 days post administration, lasting at least to about 85 days post administration, lasting at least to about 90 days post administration, lasting at least to about 95 days post administration, lasting at least to about 100 days post administration, lasting at least to about 105 days post administration, lasting at least to about 110 days post administration, lasting at least to about 115 days post administration, lasting at least to about 120 days post administration, lasting at least to about 125 days post administration, lasting at least to about 130 days post administration, or lasting at least to about 135 days post administration). The previously received vaccine can be one that the mammal (e.g., the human) received from about 21 days to about 120 months (e.g., from about 21 days to about 60 months, from about 21 days to about 36 months, from about 21 days to about 24 months, from about 21 days to about 12 months, from about 21 days to about 8 months, from about 21 days to about 6 months, from about 21 days to about 4 months, from about 21 days to about 3 months, from about 21 days to about 2 months, from about 1 month to about 120 months, from about 3 months to about 120 months, from about 6 months to about 120 months, from about 12 months to about 120 months, from about 18 months to about 120 months, from about 24 months to about 120 months, from about 36 months to about 120 months, from about 48 months to about 120 months, from about 1 month to about 60 months, from about 2 months to about 48 months, from about 3 months to about 36 months, from about 4 months to about 24 months, from about 6 months to about 18 months, from about 1 month to about 3 months, from about 3 months to about 6 months, from about 6 months to about 12 months, from about 12 months to about 24 months, from about 24 months to about 48 months, from about 36 months to about 72 months, from about 48 months to about 84 months, or from about 60 months to about 96 months) before the mammal is intranasally administered an adenovirus vector described herein (and/or nucleic acid molecules that can encode an adenovirus vector described herein).

In some cases, adenovirus vectors described herein and/or nucleic acid molecules that can encode an adenovirus vector described herein can be administered intranasally as a heterologous vaccination boost to a mammal (e.g., a human) that previously received a vaccine targeting the same pathogen (e.g., a bacterial or a viral pathogen) to increase a T cell response within the mammal. For example, adenovirus vectors described herein encoding one or more immunogens (and/or nucleic acid molecules that can encode an adenovirus vector described herein encoding one or more immunogens) can be administered intranasally as a heterologous vaccination boost to a mammal that previously received a vaccine targeting the same pathogen (e.g., a bacterial or a viral pathogen) to increase the number of activated T cells (e.g., activated Th1 T cells) within the mammal by, for example, 10, 20, 30, 40, 50, 60, 70, 80, 90, 95, or more percent.

As described herein, a SC-Ad engineered to encode one or more immunogens (e.g., a coronavirus Spike polypeptide) can be intranasally administered as a heterologous vaccination boost to a mammal (e.g., a human) that previously received a vaccine targeting the same pathogen (e.g., a bacterial or a viral pathogen) to induce IgG antibodies, IgA antibodies, and/or Th1 T cell responses (e.g., skewed Th1 T cell responses) within the mammal. For example, a SC-Ad engineered to encode a SARS-CoV-2 Spike polypeptide can be intranasally administered as a heterologous vaccination boost to a human who previously received an mRNA-based or DNA-based SARS-CoV-2 vaccine (e.g., a Pfizer®) vaccine such as Comirnaty®, an AstraZeneca® vaccine such as Vaxzevria®, a Moderna® vaccine such as Spikevax®, a Novavax® vaccine such as Nuvaxovid®, and/or Sputnik) to induce IgG antibodies, IgA antibodies, and/or Th1 T cell responses against coronaviruses within the human. The previously received SARS-CoV-2 vaccine can be one that the human received from about 21 days to about 120 months (e.g., e.g., from about 21 days to about 60 months, from about 21 days to about 36 months, from about 21 days to about 24 months, from about 21 days to about 12 months, from about 21 days to about 8 months, from about 21 days to about 6 months, from about 21 days to about 4 months, from about 21 days to about 3 months, from about 21 days to about 2 months, from about 1 month to about 120 months, from about 3 months to about 120 months, from about 6 months to about 120 months, from about 12 months to about 120 months, from about 18 months to about 120 months, from about 24 months to about 120 months, from about 36 months to about 120 months, from about 48 months to about 120 months, from about 1 month to about 60 months, from about 2 months to about 48 months, from about 3 months to about 36 months, from about 4 months to about 24 months, from about 6 months to about 18 months, from about 1 month to about 3 months, from about 3 months to about 6 months, from about 6 months to about 12 months, from about 12 months to about 24 months, from about 24 months to about 48 months, from about 36 months to about 72 months, from about 48 months to about 84 months, or from about 60 months to about 96 months) before the human is intranasally administered a SC-Ad described herein (e.g., a SC-Ad engineered to encode a SARS-CoV-2 Spike polypeptide). The previously received SARS-CoV-2 vaccine can be one that the human received at least 90 days (e.g., at least 120 days, at least 150 days before said intranasally administering, or at least 180 days) before the human is intranasally administered a SC-Ad described herein (e.g., a SC-Ad engineered to encode a SARS-CoV-2 Spike polypeptide).

Adenovirus vectors described herein (e.g., adenovirus vectors encoding one or more immunogens) and/or nucleic acid molecules that can encode an adenovirus vector described herein can be administered intranasally to any appropriate mammal (e.g., to increase an immune response against a pathogen such as a bacterial or a viral pathogen associated with the immunogen(s) encoded by the adenovirus vectors within that mammal). In some cases, the mammal can be a mammal that has not had a previous infection with a pathogen associated with an immunogen encoded by an adenovirus vector provided herein. In some cases, the mammal can be a mammal that has had a previous infection with a pathogen closely related (e.g., genetically related) to a pathogen associated with an immunogen encoded by an adenovirus vector provided herein. In some cases, the mammal can be a mammal that has an infection (e.g., an ongoing infection) with a pathogen associated with an immunogen encoded by an adenovirus vector provided herein. Examples of mammals that can be intranasally administered adenovirus vectors described herein encoding one or more immunogens (and/or nucleic acid molecules that can encode an adenovirus vector described herein encoding one or more immunogens) include, without limitation, humans, non-human primates such as monkeys, dogs, cats, horses, cows, pigs, sheep, mice, rats, rabbits, hamsters, bats, raccoons, and ferrets. In some cases, a human can be intranasally administered one or more adenovirus vectors described herein and/or nucleic acid molecules that can encode an adenovirus vector described herein to increase an immune response against a pathogen (e.g., a bacterial or a viral pathogen associated with an immunogen encoded by the adenovirus vectors).

Adenovirus vectors described herein (e.g., adenovirus vectors encoding one or more immunogens) and/or nucleic acid molecules that can encode an adenovirus vector described herein (e.g., a composition such as a vaccine composition including adenovirus vectors provided herein and/or nucleic acid molecules that can encode an adenovirus vector provided herein) can be administered intranasally (e.g., administered intranasally as a heterologous vaccination boost) to a mammal (e.g., a human) in any appropriate amount (e.g., any appropriate dose). Effective amounts can vary depending on the age and general health condition of the subject, excipient usage, the possibility of co-usage with other therapeutic treatments such as use of other agents, and the judgment of the treating physician. An effective amount of a composition containing adenovirus vectors encoding one or more immunogens (and/or nucleic acid molecules that can encode an adenovirus vector encoding one or more immunogens) can be any amount that can induce an immune response in a mammal as described herein without producing significant toxicity to the mammal. For example, an effective amount adenovirus vectors encoding one or more immunogens can be, for example, from about 10⁸ viral particles (vp) to about 10¹⁴ vp (e.g., from about 10⁸ vp to about 10¹³ vp, from about 10⁸ vp to about 10¹² vp, from about 10⁸ vp to about 10¹¹ vp, from about 10⁸ vp to about 10¹⁰ vp, from about 10⁸ vp to about 10⁹ vp, from about 10⁹ vp to about 10¹⁴ vp, from about 10¹⁰ vp to about 10¹⁴ vp, from about 10¹¹ vp to about 10¹⁴ vp, from about 10¹² vp to about 10¹⁴ vp, from about 10¹³ vp to about 10¹⁴ vp, from about 10⁹ vp to about 10¹³ vp, from about 10¹⁰ vp to about 10¹² vp, from about 10⁹ vp to about 10¹¹ vp, from about 10¹⁰ vp to about 10¹² vp, or from about 10¹ vp to about 10¹³ vp). The effective amount can remain constant or can be adjusted as a sliding scale or variable dose depending on the mammal's response to treatment. Various factors can influence the actual effective amount used for a particular application. For example, the frequency of administration and/or use of multiple treatment agents may require an increase or decrease in the actual effective amount administered.

Adenovirus vectors provided herein (e.g., adenovirus vectors encoding one or more immunogens) and/or nucleic acid molecules that can encode an adenovirus vector provided herein (e.g., a composition such as a vaccine composition including adenovirus vectors provided herein and/or nucleic acid molecules that can encode an adenovirus vector provided herein) can be administered intranasally (e.g., administered intranasally as a heterologous vaccination boost) to a mammal (e.g., a human) in any appropriate frequency. The frequency of administration can be any frequency that can induce an immune response in a mammal without producing significant toxicity to the mammal. In some cases, adenovirus vectors provided herein and/or nucleic acid molecules that can encode an adenovirus vector provided herein can be administered intranasally (e.g., administered intranasally as a heterologous vaccination boost) to a mammal once (e.g., in a single administration). In some cases, adenovirus vectors provided herein and/or nucleic acid molecules that can encode an adenovirus vector provided herein can be administered intranasally (e.g., administered intranasally as a heterologous vaccination boost) to a mammal several times (e.g., as several administrations). For example, the frequency of administration can be from about once a day to about every three days, from about once a day to about once a week, from about once a week to about every 3 weeks, from about once a week to about every 6 weeks, from about once every three months to about once every 6 months, from once every 6 months to once every 18 months, or from once every 6 months to once every year. As with the effective amount, various factors can influence the actual frequency of administration used for a particular application. For example, the effective amount and/or use of multiple treatment agents may require an increase or decrease in administration frequency.

In some cases, one or more adenovirus vectors provided herein (e.g., an adenovirus vector encoding one or more immunogens) and/or a nucleic acid molecule that can encode an adenovirus vector provided herein (e.g., a composition such as a vaccine composition including adenovirus vectors provided herein and/or nucleic acid molecules that can encode an adenovirus vector provided herein) can be administered intranasally (e.g., administered intranasally as a heterologous vaccination boost) to a mammal (e.g., a human) as the sole active ingredient to increase an immune response (e.g., to increase IgG and IgA antibody responses and/or to increase Th1 responses) against a pathogen (e.g., a bacterial or a viral pathogen associated with an immunogen encoded by the adenovirus vectors). For example, a composition containing adenovirus vectors described herein encoding one or more coronavirus immunogens (and/or nucleic acid molecules that can encode an adenovirus vector described herein encoding one or more coronavirus immunogens) can be administered intranasally (e.g., administered intranasally as a heterologous vaccination boost) to a mammal (e.g., a human) as the sole active ingredient to increase an immune response (e.g., to increase IgG and IgA antibody responses and/or to increase Th1 T cell responses) against a coronavirus.

In some cases, one or more adenovirus vectors provided herein (e.g., an adenovirus vector encoding one or more immunogens) and/or a nucleic acid molecule that can encode an adenovirus vector provided herein (e.g., a composition such as a vaccine composition including adenovirus vectors provided herein and/or nucleic acid molecules that can encode an adenovirus vector provided herein) can be administered intranasally (e.g., administered intranasally as a heterologous vaccination boost) to a mammal (e.g., a human) together with one or more (e.g., one, two, three, four, five or more) additional agents/therapies used to increase an immune response (e.g., to increase IgG and IgA antibody responses and/or to increase Th1 T cell responses) against a pathogen (e.g., a bacterial or a viral pathogen associated with an immunogen encoded by the adenovirus vectors). For example, a composition containing adenovirus vectors described herein encoding one or more coronavirus immunogens (and/or nucleic acid molecules that can encode an adenovirus vector described herein encoding one or more coronavirus immunogens) can be administered intranasally (e.g., administered intranasally as a heterologous vaccination boost) to a mammal (e.g., a human) together with one or more (e.g., one, two, three, four, five or more) additional agents/therapies used to increase an immune response (e.g., to increase IgG and IgA antibody responses and/or to increase Th1 T cell responses) against a coronavirus. In cases where one or more adenovirus vectors provided herein (e.g., an adenovirus vector encoding one or more immunogens) and/or a nucleic acid molecule that can encode an adenovirus vector provided herein (e.g., a composition such as a vaccine composition including adenovirus vectors provided herein and/or nucleic acid molecules that can encode an adenovirus vector provided herein) are used in combination with one or more additional agents/therapies used to increase an immune response (e.g., to increase IgG and IgA antibody responses and/or to increase Th1 T cell responses), the one or more adenovirus vectors provided herein (and/or a nucleic acid molecule that can encode an adenovirus vector provided herein) and the one or more additional agents/therapies can be administered at the same time (e.g., in a single composition) or independently. For example, one or more adenovirus vectors provided herein (and/or a nucleic acid molecule that can encode an adenovirus vector provided herein) can be administered first, and the one or more additional agents/therapies administered second, or vice versa.

The invention will be further described in the following examples, which do not limit the scope of the invention described in the claims.

### EXAMPLES

### Example 1: A Replicating Single-Cycle Adenovirus Vaccine Against COVID-19

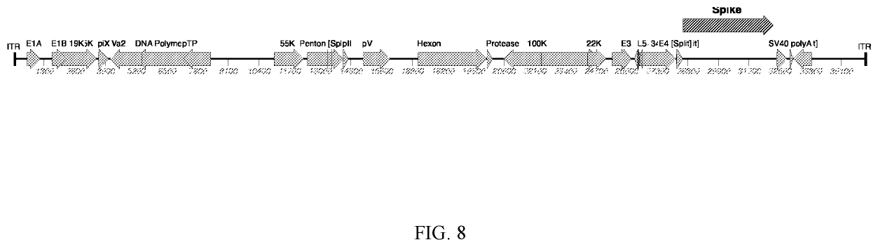

SC-Ad6-1 (a COVID-19 vaccine; FIGS. 8 and 9) was safe and well tolerated in the initial ascending dose cohorts. In addition, when SC-Ad6-1 was given as a heterologous boost of humans who received a previous mRNA COVID-19 vaccine, a strong mucosal and systemic neutralizing response against the SARS-CoV-2 spike protein was observed in a majority of the humans.

Ten healthy volunteers who had been fully vaccinated against COVID-19 for at least 3 months prior to Day 1 of SC-Ad6-1 dosing as a heterologous vaccination boost were enrolled (FIG. 2). One of the ten volunteers was removed from the analysis since that person became infected with SARS-CoV-2 two days into the study. The humans were dosed with 8.0e8 infectious units (IU) of SC-Ad6-1 via intranasal administration, and humoral and cellular immune responses were evaluated. The immunogenicity endpoints were humoral and cellular immune responses to SARS-CoV-2 as determined by (a) the presence of serum anti-SARS-CoV-2 neutralizing antibodies via a live virus assay, (b) the presence of serum IgG antibodies specific for the SARS-CoV-2 spike protein via an ELISA, (c) the presence of mucosal IgA antibodies specific for the SARS-CoV-2 spike protein via an ELISA, and (d) the presence of cytokine-producing Th1 T cells specific for the SARS-CoV-2 spike protein via or interferon-y secretion from stimulated PBMCs.

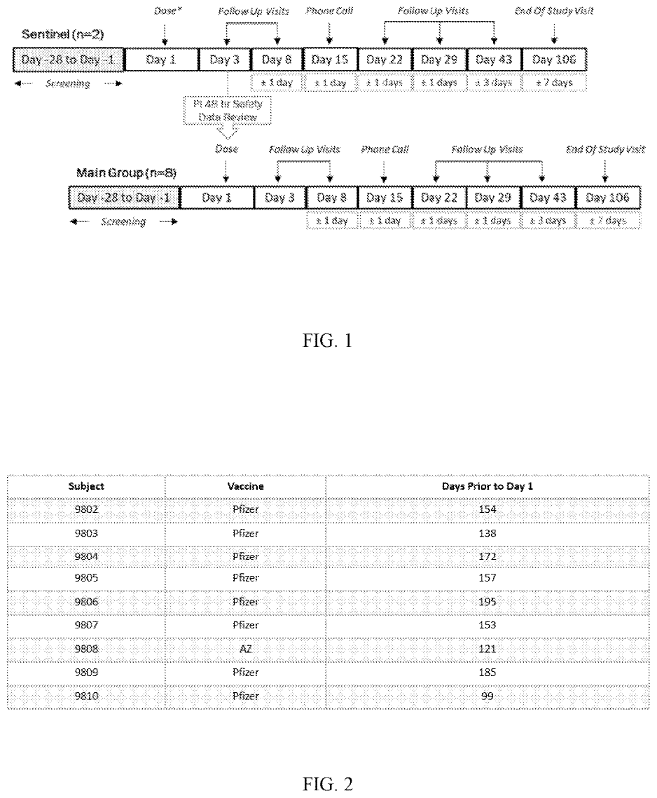

For safety reasons, the first two humans (sentinels) were dosed ahead of the remaining eight humans and at least one hour apart from each other (FIG. 1). The profile of adverse events in the sentinels was reviewed 48 hours post intranasal administration (study Day 3). No safety concerns were observed in the sentinels, and the remainder of human participants were dosed at the same dose level after at least 48 hours have elapsed following dosing of the sentinel participants (FIG. 1).

Each human participant visited the clinical facility on the day of dosing (Day 1) and on study Days 3, 8, 22, 29, and 43 for follow-up assessments of safety, immunogenicity, and pharmacodynamics. A follow-up phone call was conducted on study Day 15 to assess adverse events. The End-of-Study (EoS) visit was on study Day 106 (FIG. 1).

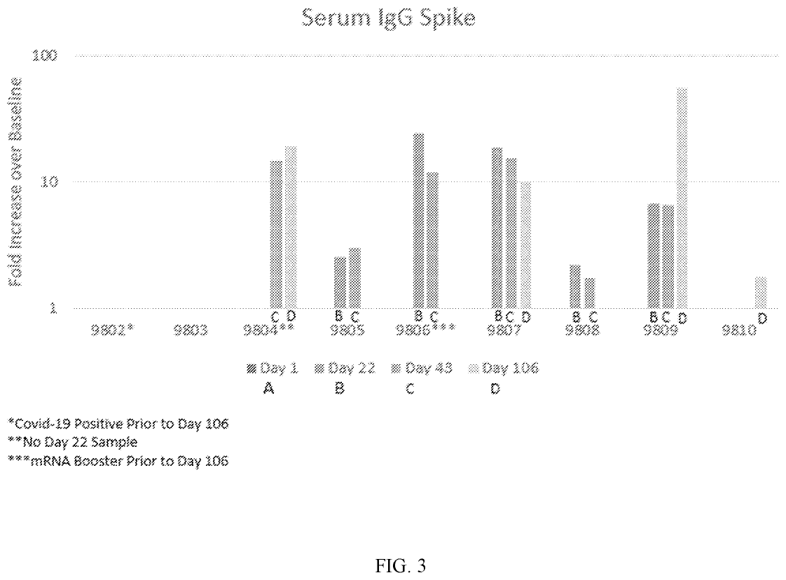

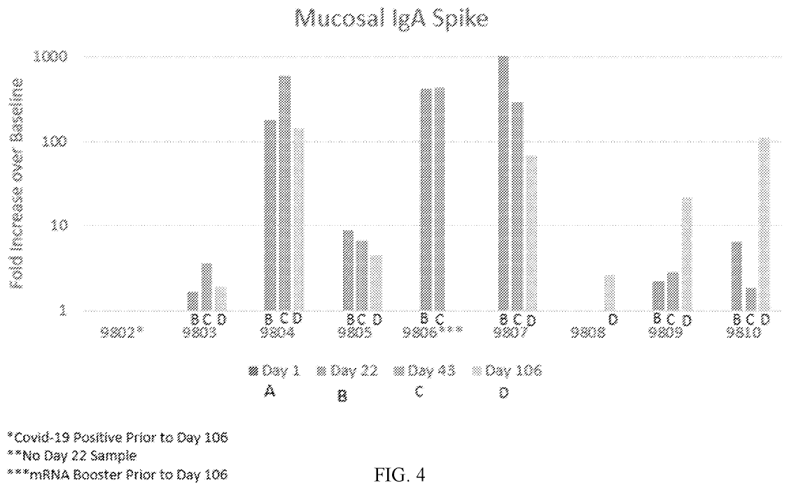

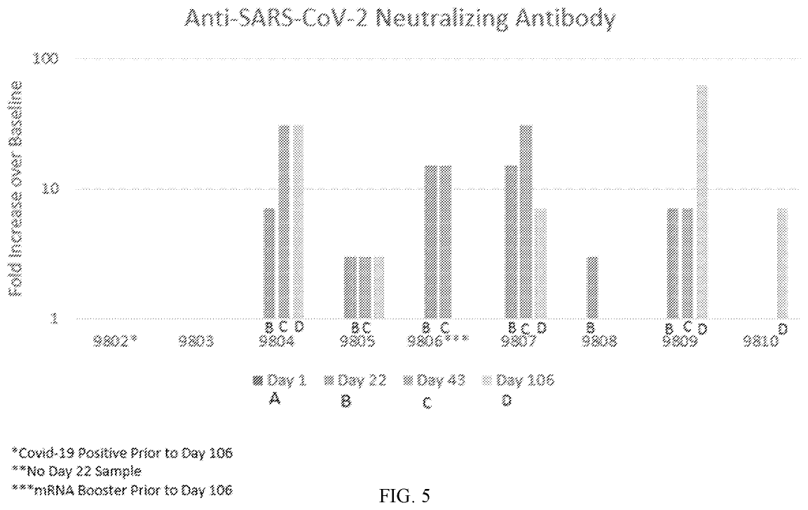

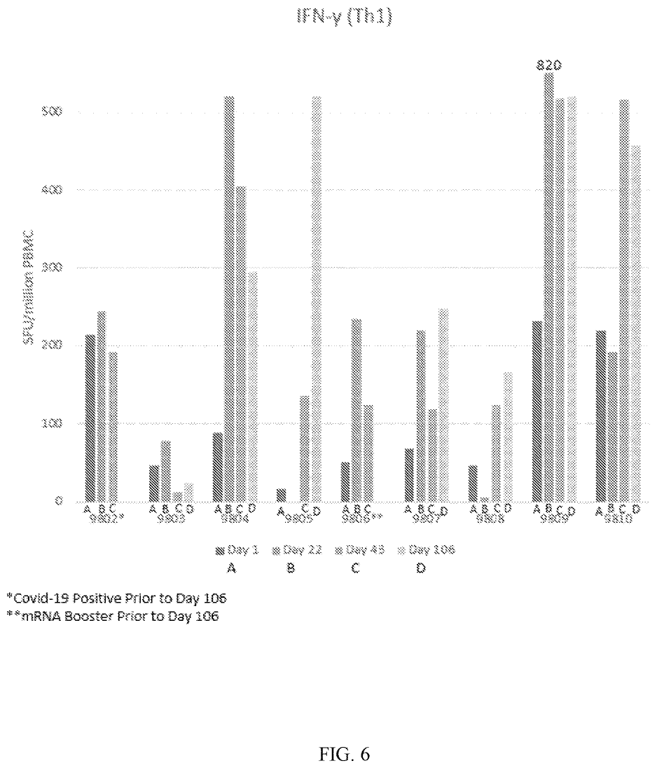

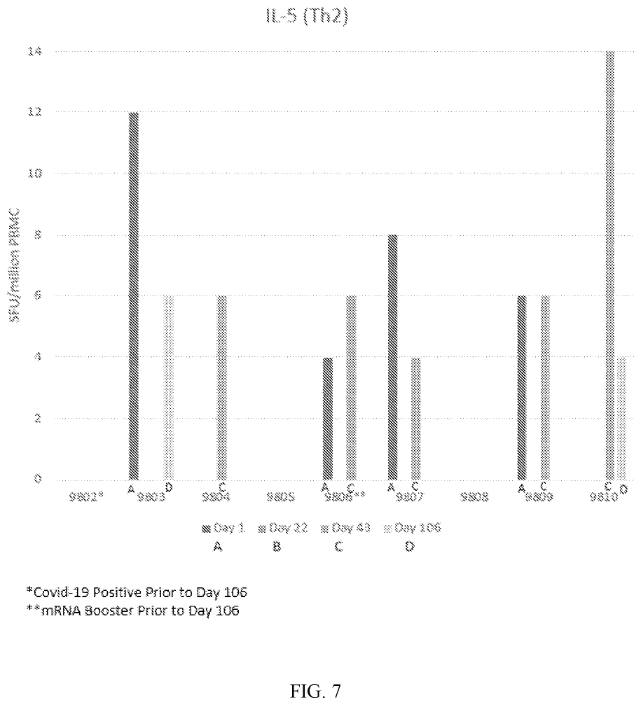

Intranasal administration of SC-Ad6-1 as a nasal spray to humans as a heterologous vaccination boost resulted in an increase in the amount of serum IgG antibodies specific for the SARS-CoV-2 spike protein as determined by an ELISA (FIG. 3), resulted in an increase in the amount of mucosal IgA antibodies specific for the SARS-CoV-2 spike protein as determined by an ELISA (FIG. 4), resulted in an increase in the amount of serum anti-SARS-CoV-2 neutralizing antibodies as determined by a live virus assay (FIG. 5), and resulted in T cells against SARS-CoV-2 spike being skewed towards a Th1 response (critical for pathogen immunity) versus a Th2 response as determined by interferon-γ secretion (FIG. 6) versus IL-5 secretion (FIG. 7) from stimulated PBMCs, respectively.

These results demonstrate that a SC-Ad designed to encode a SARS-CoV-2 immunogen can be administered intranasally as a heterologous vaccination boost to humans who received a prior COVID-19 vaccine to induce IgG and IgA antibodies against SARS-CoV-2 and Th1 T cell immune responses (e.g., Th1 skewed T cell immune responses) against SARS-CoV-2.

### Example 2: SC-Ad6-1 DNA Sequence

SEQ ID NO: 1
CGCCATCATCAATAATATACCTTATTTTGGATTGAAGCCAATATGATAATGAGGGGGTGGAG
TTTGTGACGTGGCGCGGGGCGTGGGAACGGGGGGGGTGACGTAGTAGTGTGGCGGAAGTGTG
ATGTTGTAAGTGTGGCGGAACACATGTAAGCGCCGGATGTGGTAAAAGTGACGTTTTTGGTG
TGCGCCGGTGTACACGGGAAGTGACAATTTTCGCGCGGTTTTAGGCGGATGTTGTAGTAAAT
TTGGGCGTAACCAAGTAATATTTGGCCATTTTCGCGGGAAAACTGAATAAGAGGAAGTGAAA
TCTGAATAATTCTGTGTTACTCATAGCGCGTAATATTTGTCTAGGGCCGCGGGGACTTTGAC
CGTTTACGTGGAGACTCGCCCAGGTGTTTTTCTCAGGTGTTTTCCGCGTTCCGGGTCAAAGT
TGGCGTTTTATTATTATAGTCAGCTGACGCGCAGTGTATTTATACCCGGTGAGTTCCTCAAG
AGGCCACTCTTGAGTGCCAGCGAGTAGAGTTTTCTCCTCCGAGCCGCTCCGACACCGGGACT
GAAAATGAGACATATTATCTGCCACGGAGGTGTTATTACCGAAGAAATGGCCGCCAGTCTTT
TGGACCAGCTGATCGAAGAGGTACTGGCTGATAATCTTCCACCTCCTAGCCATTTTGAACCA
CCTACCCTTCACGAACTGTATGATTTAGACGTGACGGCCCCCGAAGATCCCAACGAGGAGGC
GGTTTCGCAGATTTTTCCCGAGTCTGTAATGTTGGCGGTGCAGGAAGGGATTGACTTATTCA
CTTTTCCGCCGGCGCCCGGTTCTCCGGAGCCGCCTCACCTTTCCCGGCAGCCCGAGCAGCCG
GAGCAGAGAGCCTTGGGTCCGGTTTCTATGCCAAACCTTGTGCCGGAGGTGATCGATCTTAC
CTGCCACGAGGCTGGCTTTCCACCCAGTGACGACGAGGATGAAGAGGGTGAGGAGTTTGTGT
TAGATTATGTGGAGCACCCCGGGCACGGTTGCAGGTCTTGTCATTATCACCGGAGGAATACG
GGGGACCCAGATATTATGTGTTCGCTTTGCTATATGAGGACCTGTGGCATGTTTGTCTACAG
TAAGTGAAAAATTATGGGCAGTGGGTGATAGAGTGGTGGGTTTGGTGTGGTAATTTTTTTTT
TAATTTTTACAGTTTTGTGGTTTAAAGAATTTTGTATTGTGATTTTTTAAAAGGTCCTGTGT
CTGAACCTGAGCCTGAGCCCGAGCCAGAACCGGAGCCTGCAAGACCTACCCGGCGTCCTAAA
TTGGTGCCTGCTATCCTGAGACGCCCGACATCACCTGTGTCTAGAGAATGCAATAGTAGTAC
GGATAGCTGTGACTCCGGTCCTTCTAACACACCTCCTGAGATACACCCGGTGGTCCCGCTGT
GCCCCATTAAACCAGTTGCCGTGAGAGTTGGTGGGCGTCGCCAGGCTGTGGAATGTATCGAG
GACTTGCTTAACGAGTCTGGGCAACCTTTGGACTTGAGCTGTAAACGCCCCAGGCCATAAGG
TGTAAACCTGTGATTGCGTGTGTGGTTAACGCCTTTGTTTGCTGAATGAGTTGATGTAAGTT
TAATAAAGGGTGAGATAATGTTTAACTTGCATGGCGTGTTAAATGGGGCGGGGCTTAAAGGG
TATATAATGCGCCGTGGGCTAATCTTGGTTACATCTGACCTCATGGAGGCTTGGGAGTGITT
GGAAGATTTTTCTGCTGTGCGTAACTTGCTGGAACAGAGCTCTAACAGTACCTCTTGGTTTT
GGAGGTTTCTGTGGGGCTCCTCCCAGGCAAAGTTAGTCTGCAGAATTAAGGAGGATTACAAG
TGGGAATTTGAAGAGCTTTTGAAATCCTGTGGTGAGCTGTTTGATTCTTTGAATCTGGGTCA
CCAGGCGCTTTTCCAAGAGAAGGTCATCAAGACTTTGGATTTTTCCACACCGGGGCGCGCTG
CGGCTGCTGTTGCTTTTTTGAGTTTTATAAAGGATAAATGGAGCGAAGAAACCCATCTGAGC
GGGGGGTACCTGCTGGATTTTCTGGCCATGCATCTGTGGAGAGCGGTGGTGAGACACAAGAA
TCGCCTGCTACTGTTGTCTTCCGTCCGCCCGGCAATAATACCGACGGAGGAGCAACAGCAGG
AGGAAGCCAGGCGGCGGCGGCGGCAGGAGCAGAGCCCATGGAACCCGAGAGCCGGCCTGGAC
CCTCGGGAATGAATGTTGTACAGGTGGCTGAACTGTTTCCAGAACTGAGACGCATTTTAACC
ATTAACGAGGATGGGCAGGGGCTAAAGGGGGTAAAGAGGGAGCGGGGGGCTTCTGAGGCTAC
AGAGGAGGCTAGGAATCTAACTTTTAGCTTAATGACCAGACACCGTCCTGAGTGTGTTACTT
TTCAGCAGATTAAGGATAATTGCGCTAATGAGCTTGATCTGCTGGCGCAGAAGTATTCCATA
AAGCAGCTGACCACTTACTGGCTGCAGCCAGGGGATGATTTTGAGGAGGCTATTAGGGTATA
TGCAAAGGTGGCACTTAGGCCAGATTGCAAGTACAAGATTAGCAAACTTGTAAATATCAGGA
ATTGTTGCTACATTTCTGGGAACGGGGCCGAGGTGGAGATAGATACGGAGGATAGGGTGGCC
TTTAGATGTAGCATGATAAATATGTGGCCGGGGGTGCTTGGCATGGACGGGGTGGTTATTAT
GAATGTGAGGTTTACTGGTCCCAATTTTAGCGGTACGGTTTTCCTGGCCAATACCAATCTTA
TCCTACACGGTGTAAGCTTCTATGGGTTTAACAATACCTGTGTGGAAGCCTGGACCGATGTA
AGGGTTCGGGGCTGTGCCTTTTACTGCTGCTGGAAGGGGGTGGTGTGTCGCCCCAAAAGCAG
GGCTTCAATTAAGAAATGCCTGTTTGAAAGGTGTACCTTGGGTATCCTGTCTGAGGGTAACT
CCAGGGTGCGCCACAATGTGGCCTCCGACTGTGGTTGCTTTATGCTAGTGAAAAGCGTGGCT
GTGATTAAGCATAACATGGTGTGTGGCAACTGCGAGGACAGGGCCTCTCAGATGCTGACCTG
CTCGGACGGCAACTGTCACTTGCTGAAGACCATTCACGTAGCCAGCCACTCTCGCAAGGCCT
GGCCAGTGTTTGAGCACAACATACTGACCCGCTGTTCCTTGCATTTGGGTAACAGGAGGGGG
GTGTTCCTACCTTACCAATGCAATTTGAGTCACACTAAGATATTGCTTGAGCCCGAGAGCAT
GTCCAAGGTGAACCTGAACGGGGTGTTTGACATGACCATGAAGATCTGGAAGGTGCTGAGGT
ACGATGAGACCCGCACCAGGTGCAGACCCTGCGAGTGTGGCGGTAAACATATTAGGAACCAG
CCTGTGATGCTGGATGTGACCGAGGAGCTGAGGCCCGATCACTTGGTGCTGGCCTGCACCCG
CGCTGAGTTTGGCTCTAGCGATGAAGATACAGATTGAGGTACTGAAATGTGTGGGCGTGGCT
TAAGGGTGGGAAAGAATATATAAGGTGGGGGTCTCATGTAGTTTTGTATCTGTTTTGCAGCA
GCCGCCGCCATGAGCGCCAACTCGTTTGATGGAAGCATTGTGAGCTCATATTTGACAACGCG
CATGCCCCCATGGGCCGGGGTGCGTCAGAATGTGATGGGCTCCAGCATTGATGGTCGCCCCG
TCCTGCCCGCAAACTCTACTACCTTGACCTACGAGACCGTGTCTGGAACGCCGTTGGAGACT
GCAGCCTCCGCCGCCGCTTCAGCCGCTGCAGCCACCGCCCGCGGGATTGTGACTGACTTTGC
TTTCCTGAGCCCGCTTGCAAGCAGTGCAGCTTCCCGTTCATCCGCCCGCGATGACAAGTTGA
CGGCTCTTTTGGCACAATTGGATTCTTTGACCCGGGAACTTAATGTCGTTTCTCAGCAGCTG
TTGGATCTGCGCCAGCAGGTTTCTGCCCTGAAGGCTTCCTCCCCTCCCAATGCGGTTTAAAA
CATAAATAAAAACCAGACTCTGTTTGGATTTGGATCAAGCAAGTGTCTTGCTGTCTTTATTT
AGGGGTTTTGCGCGCGCGGTAGGCCCGGGACCAGCGGTCTCGGTCGTTGAGGGTCCTGTGTA
TTTTTTCCAGGACGTGGTAAAGGTGACTCTGGATGTTCAGATACATGGGCATAAGCCCGTCT
CTGGGGTGGAGGTAGCACCACTGCAGAGCTTCATGCTGCGGGGTGGTGTTGTAGATGATCCA
GTCGTAGCAGGAGCGCTGGGCGTGGTGCCTAAAAATGTCTTTCAGTAGCAAGCTGATTGCCA
GGGGCAGGCCCTTGGTGTAAGTGTTTACAAAGCGGTTAAGCTGGGATGGGTGCATACGTGGG
GATATGAGATGCATCTTGGACTGTATTTTTAGGTTGGCTATGTTCCCAGCCATATCCCTCCG
GGGATTCATGTTGTGCAGAACCACCAGCACAGTGTATCCGGTGCACTTGGGAAATTTGTCAT
GTAGCTTAGAAGGAAATGCGTGGAAGAACTTGGAGACGCCCTTGTGACCTCCAAGATTTTCC
ATGCATTCGTCCATAATGATGGCAATGGGCCCACGGGCGGCGGCCTGGGCGAAGATATTTCT
GGGATCACTAACGTCATAGTTGTGTTCCAGGATGAGATCGTCATAGGCCATTTTTACAAAGC
GCGGGCGGAGGGTGCCAGACTGCGGTATAATGGTTCCATCCGGCCCAGGGGCGTAGTTACCC
TCACAGATTTGCATTTCCCACGCTTTGAGTTCAGATGGGGGGATCATGTCTACCTGCGGGGC
GATGAAGAAAACCGTTTCCGGGGTAGGGGAGATCAGCTGGGAAGAAAGCAGGTTCCTAAGCA
GCTGCGACTTACCGCAGCCGGTGGGCCCGTAAATCACACCTATTACCGGCTGCAACTGGTAG
TTAAGAGAGCTGCAGCTGCCGTCATCCCTGAGCAGGGGGGCCACTTCGTTAAGCATGTCCCT
GACTTGCATGTTTTCCCTGACCAAATCCGCCAGAAGGCGCTCGCCGCCCAGCGATAGCAGTT
CTTGCAAGGAAGCAAAGTTTTTCAACGGTTTGAGGCCGTCCGCCGTAGGCATGCTTTTGAGC
GTTTGACCAAGCAGTTCCAGGCGGTCCCACAGCTCGGTCACGTGCTCTACGGCATCTCGATC
CAGCATATCTCCTCGTTTCGCGGGTTGGGGCGGCTTTCGCTGTACGGCAGTAGTCGGTGCTC
GTCCAGACGGGCCAGGGTCATGTCTTTCCACGGGCGCAGGGTCCTCGTCAGCGTAGTCTGGG
TCACGGTGAAGGGGTGCGCTCCGGGTTGCGCGCTGGCCAGGGTGCGCTTGAGGCTGGTCCTG
CTGGTGCTGAAGCGCTGCCGGTCTTCGCCCTGCGCGTCGGCCAGGTAGCATTTGACCATGGT
GTCATAGTCCAGCCCCTCCGCGGCGTGGCCCTTGGCGCGCAGCTTGCCCTTGGAGGAGGCGC
CGCACGAGGGGCAGTGCAGACTTTTAAGGGCGTAGAGCTTGGGCGCGAGAAATACCGATTCC
GGGGAGTAGGCATCCGCGCCGCAGGCCCCGCAGACGGTCTCGCATTCCACGAGCCAGGTGAG
CTCTGGCCGTTCGGGGTCAAAAACCAGGTTTCCCCCATGCTTTTTGATGCGTTTCTTACCTC
TGGTTTCCATGAGCCGGTGTCCACGCTCGGTGACGAAAAGGCTGTCCGTGTCCCCGTATACA
GACTTGAGAGGCCTGTCCTCGAGCGGTGTTCCGCGGTCCTCCTCGTATAGAAACTCGGACCA
CTCTGAGACGAAGGCTCGCGTCCAGGCCAGCACGAAGGAGGCTAAGTGGGAGGGGTAGCGGT
CGTTGTCCACTAGGGGGTCCACTCGCTCCAGGGTGTGAAGACACATGTCGCCCTCTTCGGCA
TCAAGGAAGGTGATTGGTTTATAGGTGTAGGCCACGTGACCGGGTGTTCCTGAAGGGGGGCT
ATAAAAGGGGGTGGGGGCGCGTTCGTCCTCACTCTCTTCCGCATCGCTGTCTGCGAGGGCCA
GCTGTTGGGGTGAGTACTCCCTCTCAAAAGCGGGCATGACTTCTGCGCTAAGATTGTCAGTT
TCCAAAAACGAGGAGGATTTGATATTCACCTGGCCCGCGGTGATGCCTTTGAGGGTGGCCGC
GTCCATCTGGTCAGAAAAGACAATCTTTTTGTTGTCAAGCTTGGTGGCAAACGACCCGTAGA
GGGCGTTGGACAGCAACTTGGCGATGGAGCGCAGGGTTTGGTTTTTGTCGCGATCGGCGCGC
TCCTTGGCCGCGATGTTTAGCTGCACGTATTCGCGCGCAACGCACCGCCATTCGGGAAAGAC
GGTGGTGCGCTCGTCGGGCACTAGGTGCACGCGCCAACCGCGGTTGTGCAGGGTGACAAGGT
CAACGCTGGTGGCTACCTCTCCGCGTAGGCGCTCGTTGGTCCAGCAGAGGCGGCCGCCCTTG
CGCGAGCAGAATGGCGGTAGTGGGTCTAGCTGCGTCTCGTCCGGGGGGTCTGCGTCCACGGT
AAAGACCCCGGGCAGCAGGCGCGCGTCGAAGTAGTCTATCTTGCATCCTTGCAAGTCTAGCG
CCTGCTGCCATGCGCGGGCGGCAAGCGCGCGCTCGTATGGGTTGAGTGGGGGACCCCATGGC
ATGGGGTGGGTGAGCGCGGAGGCGTACATGCCGCAAATGTCGTAAACGTAGAGGGGCTCTCT
GAGTATTCCAAGATATGTAGGGTAGCATCTTCCACCGCGGATGCTGGCGCGCACGTAATCGT
ATAGTTCGTGCGAGGGAGCGAGGAGGTCGGGACCGAGGTTGCTACGGGCGGGCTGCTCTGCT
CGGAAGACTATCTGCCTGAAGATGGCATGTGAGTTGGATGATATGGTTGGACGCTGGAAGAC
GTTGAAGCTGGCGTCTGTGAGACCTACCGCGTCACGCACGAAGGAGGCGTAGGAGTCGCGCA
GCTTGTTGACCAGCTCGGCGGTGACCTGCACGTCTAGGGCGCAGTAGTCCAGGGTTTCCTTG
ATGATGTCATACTTATCCTGTCCCTTTTTTTTCCACAGCTCGCGGTTGAGGACAAACTCTTC
GCGGTCTTTCCAGTACTCTTGGATCGGAAACCCGTCGGCCTCCGAACGGTAAGAGCCTANCA
TGTAGAACTGGTTGACGGCCTGGTAGGCGCAGCATCCCTTTTCTACGGGTAGCGCGTATGCC
TGCGCGGCCTTCCGGAGCGAGGTGTGGGTGAGCGCAAAGGTGTCCCTAACCATGACTTTGAG
GTACTGGTATTTGAAGTCAGTGTCGTCGCATCCGCCCTGCTCCCAGAGCAAAAAGTCCGTGC
GCTTTTTGGAACGCGGGTTTGGCAGGGCGAAGGTGACATCGTTGAAGAGTATCTTTCCCGCG
CGAGGCATAAAGTTGCGTGTGATGCGGAAGGGTCCCGGCACCTCGGAACGGTTGTTAATTAC
CTGGGCGGCGAGCACGATCTCGTCAAAGCCGTTGATGTTGTGGCCCACAATGTAAAGTTCCA
AGAAGCGCGGGATGCCCTTGATGGAAGGCAATTTTTTAAGTTCCTCGTAGGTGAGCTCTTCA
GGGGAGCTGAGCCCGTGCTCTGAAAGGGCCCAGTCTGCAAGATGAGGGTTGGAAGCGACGAA
TGAGCTCCACAGGTCACGGGCCATTAGCATTTGCAGGTGGTCGCGAAAGGTCCTAAACTGGC
GACCTATGGCCATTTTTTCTGGGGTGATGCAGTAGAAGGTAAGCGGGTCTTGTTCCCAGCGG
TCCCATCCAAGGTCCGCGGCTAGGTCTCGCGCGGCGGTCACTAGAGGCTCATCTCCGCCGAA
CTTCATGACCAGCATGAAGGGCACGAGCTGCTTCCCAAAGGCCCCCATCCAAGTATAGGTCT
CTACATCGTAGGTGACAAAGAGACGCTCGGTGCGAGGATGCGAGCCGATCGGGAAGAACTGG
ATCTCCCGCCACCAGTTGGAGGAGTGGCTGTTGATGTGGTGAAAGTAGAAGTCCCTGCGACG
GGCCGAACACTCGTGCTGGCTTTTGTAAAAACGTGCGCAGTACTGGCAGCGGTGCACGGGCT
GTACATCCTGCACGAGGTTGACCTGACGACCGCGCACAAGGAAGCAGAGTGGGAATTTGAGC
CCCTCGCCTGGCGGGTTTGGCTGGTGGTCTTCTACTTCGGCTGCTTGTCCTTGACCGTCTGG
CTGCTCGAGGGGAGTTACGGTGGATCGGACCACCACGCCGCGCGAGCCCAAAGTCCAGATGT
CCGCGCGCGGCGGTCGGAGCTTGATGACAACATCGCGCAGATGGGAGCTGTCCATGGTCTGG
AGCTCCCGCGGCGTCAGGTCAGGCGGGAGCTCCTGCAGGTTTACCTCGCATAGCCGGGTCAG
GGCGCGGGCTAGGTCCAGGTGATACCTGATTTCCAGGGGCTGGTTGGTGGCGGCGTCGATGG
CTTGCAAGAGGCCGCATCCCCGCGGCGCGACTACGGTACCGCGCGGCGGGCGGTGGGCCGCG
GGGGTGTCCTTGGATGATGCATCTAAAAGCGGTGACGCGGGCGGGCCCCCGGAGGTAGGGGG
GGCTCGGGACCCGCCGGGAGAGGGGGCAGGGGCACGTCGGCGCCGCGCGCGGGCAGGAGCTG
GTGCTGCGCGCGGAGGTTGCTGGCGAACGCGACGACGCGGCGGTTGATCTCCTGAATCTGGC
GCCTCTGCGTGAAGACGACGGGCCCGGTGAGCTTGAACCTGAAAGAGAGTTCGACAGAATCA
ATTTCGGTGTCGTTGACGGCGGCCTGGCGCAAAATCTCCTGCACGTCTCCTGAGTTGTCTTG
ATAGGCGATCTCGGCCATGAACTGCTCGATCTCTTCCTCCTGGAGATCTCCGCGTCCGGCTC
GCTCCACGGTGGCGGCGAGGTCGTTGGAGATGCGGGCCATGAGCTGCGAGAAGGCGTTGAGG
CCTCCCTCGTTCCAGACGCGGCTGTAGACCACGCCCCCTTCGGCATCGCGGGCGCGCATGAC
CACCTGCGCGAGATTGAGCTCCACGTGCCGGGCGAAGACGGCGTAGTTTCGCAGGCGCTGAA
AGAGGTAGTTGAGGGTGGTGGCGGTGTGTTCTGCCACGAAGAAGTACATAACCCAGCGCCGC
AACGTGGATTCGTTGATATCCCCCAAGGCCTCAAGGCGCTCCATGGCCTCGTAGAAGTCCAC
GGCGAAGTTGAAAAACTGGGAGTTGCGCGCCGACACGGTTAACTCCTCCTCCAGAAGACGGA
TGAGCTCGGCGACAGTGTCGCGCACCTCGCGCTCAAAGGCTACAGGGGCCTCTTCTTCTTCT
TCAATCTCCTCTTCCATAAGGGCCTCCCCTTCTTCTTCTTCTGGCGGCGGTGGGGGAGGGGG
GACACGGCGGCGACGACGGCGCACCGGGAGGCGGTCGACAAAGCGCTCGATCATCTCCCCGC
GGCGACGGCGCATGGTCTCGGTGACGGCGCGGCCGTTCTCGCGGGGGCGCAGTTGGAAGACG
CCGCCCGTCATGTCCCGGTTATGGGTTGGCGGGGGGCTGCCGTGCGGCAGGGATACGGCGCT
AACGATGCATCTCAACAATTGTTGTGTAGGTACTCCGCCACCGAGGGACCTGAGCGAGTCCG
CATCGACCGGATCGGAAAACCTCTCGAGAAAGGCGTCTAACCAGTCACAGTCGCAAGGTAGG
CTGAGCACCGTGGCGGGCGGCAGCGGGCGGCGGTCGGGGTTGTTTCTGGCGGAGGTGCTGCT
GATGATGTAATTAAAGTAGGCGGTCTTGAGACGGCGGATGGTCGACAGAAGCACCATGTCCT
TGGGTCCGGCCTGCTGAATGCGCAGGCGGTCGGCCATGCCCCAGGCTTCGTTTTGACATCGG
CGCAGGTCTTTGTAGTAGTCTTGCATGAGCCTTTCTACCGGCACTTCTTCTTCTCCTTCCTC
TTGTCCTGCATCTCTTGCATCTATCGCTGCGGCGGCGGCGGAGTTTGGCCGTAGGTGGCGCC
CTCTTCCTCCCATGCGTGTGACCCCGAAGCCCCTCATCGGCTGAAGCAGGGCCAGGTCGGCG
ACAACGCGCTCGGCTAATATGGCCTGCTGCACCTGCGTGAGGGTAGACTGGAAGTCGTCCAT
GTCCACAAAGCGGTGGTATGCGCCCGTGTTGATGGTGTAAGTGCAGTTGGCCATAACGGACC
AGTTAACGGTCTGGTGACCCGGCTGCGAGAGCTCGGTGTACCTGAGACGCGAGTAAGCCCTT
GAGTCAAAGACGTAGTCGTTGCAAGTCCGCACCAGGTACTGGTATCCCACCAAAAAGTGCGG
CGGCGGCTGGCGGTAGAGGGGCCAGCGTAGGGTGGCCGGGGCTCCGGGGGCGAGGTCTTCCA
ACATAAGGCGATGATATCCGTAGATGTACCTGGACATCCAGGTGATGCCGGCGGCGGTGGTG
GAGGCGCGCGGAAAGTCACGGACGCGGTTCCAGATGTTGCGCAGCGGCAAAAAGTGCTCCAT
GGTCGGGACGCTCTGGCCGGTCAGGCGCGCGCAGTCGTTGACGCTCTAGACCGTGCAAAAGG
AGAGCCTGTAAGCGGGCACTCTTCCGTGGTCTGGTGGATAAATTCGCAAGGGTATCATGGCG
GACGACCGGGGTTCGAACCCCGGATCCGGCCGTCCGCCGTGATCCATGCGGTTACCGCCCGC
GTGTCGAACCCAGGTGTGCGACGTCAGACAACGGGGGAGCGCTCCTTTTGGCTTCCTTCCAG
GCGCGGCGGATGCTGCGCTAGCTTTTTTGGCCACTGGCCGCGCGCGGCGTAAGCGGTTAGGC
TGGAAAGCGAAAGCATTAAGTGGCTCGCTCCCTGTAGCCGGAGGGTTATTTTCCAAGGGTTG
AGTCGCGGGACCCCCGGTTCGAGTCTCGGGCCGGCCGGACTGCGGCGAACGGGGGTTTGCCT
CCCCGTCATGCAAGACCCCGCTTGCAAATTCCTCCGGAAACAGGGACGAGCCCCTTTTTTGC
TTTTCCCAGATGCATCCGGTGCTGCGGCAGATGCGCCCCCCTCCTCAGCAGCGGCAAGAGCA
AGAGCAGCGGCAGACATGCAGGGCACCCTCCCCTCCTCCTACCGCGTCAGGAGGGGCGACAT
CCGCGGTTGACGCGGCAGCAGATGGTGATTACGAACCCCCGCGGCGCCGGGCCCGGCACTAC
CTGGACTTGGAGGAGGGCGAGGGCCTGGCGCGGCTAGGAGCGCCCTCTCCTGAGCGGTACCC
AAGGGTGCAGCTGAAGCGTGATACGCGTGAGGCGTACGTGCCGCGGCAGAACCTGTTTCGCG
ACCGCGAGGGAGAGGAGCCCGAGGAGATGCGGGATCGAAAGTTCCACGCAGGGCGCGAGCTG
CGGCATGGCCTGAATCGCGAGCGGTTGCTGCGCGAGGAGGACTTTGAGCCCGACGCGCGAAC
CGGGATTAGTCCCGCGCGCGCACACGTGGCGGCCGCCGACCTGGTAACCGCATACGAGCAGA
CGGTGAACCAGGAGATTAACTTTCAAAAAAGCTTTAACAACCACGTGCGTACGCTTGTGGCG
CGCGAGGAGGTGGCTATAGGACTGATGCATCTGTGGGACTTTGTAAGCGCGCTGGAGCAAAA
CCCAAATAGCAAGCCGCTCATGGCGCAGCTGTTCCTTATAGTGCAGCACAGCAGGGACAACG
AGGCATTCAGGGATGCGCTGCTAAACATAGTAGAGCCCGAGGGCCGCTGGCTGCTCGATTTG
ATAAACATCCTGCAGAGCATAGTGGTGCAGGAGCGCAGCTTGAGCCTGGCTGACAAGGTGGC
CGCCATCAACTATTCCATGCTTAGCCTGGGCAAGTTTTACGCCCGCAAGATATACCATACCC
CTTACGTTCCCATAGACAAGGAGGTAAAGATCGAGGGGTTCTACATGCGCATGGCGCTGAAG
GTGCTTACCTTGAGCGACGACCTGGGCGTTTATCGCAACGAGCGCATCCACAAGGCCGTGAG
CGTGAGCCGGCGGCGCGAGCTCAGCGACCGCGAGCTGATGCACAGCCTGCAAAGGGCCCTGG
CTGGCACGGGCAGCGGCGATAGAGAGGCCGAGTCCTACTTTGACGCGGGCGCTGACCTGCGC
TGGGCCCCAAGCCGACGCGCCCTGGAGGCAGCTGGGGCCGGACCTGGGCTGGCGGTGGCACC
CGCGCGCGCTGGCAACGTCGGCGGCGTGGAGGAATATGACGAGGACGATGAGTACGAGCCAG
AGGACGGCGAGTACTAAGCGGTGATGTTTCTGATCAGTCGCGGCCGCGATATCGCTAGCGAA
GTTCCTATTCTCTAGAAAGTATAGGAACTTCGGATCCTCTAGAGTCGAAAAAAAAAAAGCAT
GATGCAAAATAAAAAACTCACCAAGGCCATGGCACCGAGCGTTGGTTTTCTTGTATTCCCCT
TAGTATGCGGCGCGCGGCGATGTATGAGGAAGGTCCTCCTCCCTCCTACGAGAGTGTGGTGA
GCGCGGCGCCAGTGGCGGCGGCGCTGGGTTCTCCCTTCGATGCTCCCCTGGACCCGCCGTTT
GTGCCTCCGCGGTACCTGCGGCCTACCGGGGGGAGAAACAGCATCCGTTACTCTGAGTTGGC
ACCCCTATTCGACACCACCCGTGTGTACCTGGTGGACAACAAGTCAACGGATGTGGCATCCC
TGAACTACCAGAACGACCACAGCAACTTTCTGACCACGGTCATTCAAAACAATGACTACAGC
CCGGGGGAGGCAAGCACACAGACCATCAATCTTGACGACCGGTCGCACTGGGGCGGCGACCT
GAAAACCATCCTGCATACCAACATGCCAAATGTGAACGAGTTCATGTTTACCAATAAGTTTA
AGGCGCGGGTGATGGTGTCGCGCTTGCCTACTAAGGACAATCAGGTGGAGCTGAAATACGAG
TGGGTGGAGTTCACGCTGCCCGAGGGCAACTACTCCGAGACCATGACCATAGACCTTATGAA
CAACGCGATCGTGGAGCACTACTTGAAAGTGGGCAGACAGAACGGGGTTCTGGAAAGCGACA
TCGGGGTAAAGTTTGACACCCGCAACTTCAGACTGGGGTTTGACCCCGTCACTGGTCTTGTC
ATGCCTGGGGTATATACAAACGAAGCCTTCCATCCAGACATCATTTTGCTGCCAGGATGCGG
GGTGGACTTCACCCACAGCCGCCTGAGCAACTTGTTGGGCATCCGCAAGCGGCAACCCTTCC
AGGAGGGCTTTAGGATCACCTACGATGATCTGGAGGGTGGTAACATTCCCGCACTGTTGGAT
GTGGACGCCTACCAGGCGAGCTTGAAAGATGACACCGAACAGGGCGGGGGTGGCGCAGGCGG
CAGCAACAGCAGTGGCAGCGGCGCGGAAGAGAACTCCAACGCGGCAGCCGCGGCAATGCAGC
CGGTGGAGGACATGAACGATCATGCCATTCGCGGCGACACCTTTGCCACACGGGCTGAGGAG
AAGCGCGCTGAGGCCGAAGCAGCGGCCGAAGCTGCCGCCCCCGCTGCGCAACCCGAGGTCGA
GAAGCCTCAGAAGAAACCGGTGATCAAACCCCTGACAGAGGACAGCAAGAAACGCAGTTACA
ACCTAATAAGCAATGACAGCACCTTCACCCAGTACCGCAGCTGGTACCTTGCATACAACTAC
GGCGACCCTCAGACCGGAATCCGCTCATGGACCCTGCTTTGCACTCCTGACGTAACCTGCGG
CTCGGAGCAGGTCTACTGGTCGTTGCCAGACATGATGCAAGACCCCGTGACCTTCCGCTCCA
CGCGCCAGATCAGCAACTTTCCGGTGGTGGGCGCCGAGCTGTTGCCCGTGCACTCCAAGAGC
TTCTACAACGACCAGGCCGTCTACTCCCAACTCATCCGCCAGTTTACCTCTCTGACCCACGT
GTTCAATCGCTTTCCCGAGAACCAGATTTTGGCGCGCCCGCCAGCCCCCACCATCACCACCG
TCAGTGAAAACGTTCCTGCTCTCACAGATCACGGGACGCTACCGCTGCGCAACAGCATCGGA
GGAGTCCAGCGAGTGACCATTACTGACGCCAGACGCCGCACCTGCCCCTACGTTTACAAGGC
CCTGGGCATAGTCTCGCCGCGCGTCCTATCGAGCCGCACTTTTTGAGCAAGCATGTCCATCC
TTATATCGCCCAGCAATAACACAGGCTGGGGCCTGCGCTTCCCAAGCAAGATGTTTGGCGGG
GCCAAGAAGCGCTCCGACCAACACCCAGTGCGCGTGCGCGGGCACTACCGCGCGCCCTGGGG
CGCGCACAAACGCGGCCGCACTGGGCGCACCACCGTCGATGACGCCATCGACGCGGTGGTGG
AGGAGGCGCGCAACTACACGCCCACGCCGCCGCCAGTGTCCACCGTGGACGCGGCCATTCAG
ACCGTGGTGCGCGGAGCCCGGCGCTACGCTAAAATGAAGAGACGGCGGAGGCGCGTAGCACG
TCGCCACCGCCGCCGACCCGGCACTGCCGCCCAACGCGCGGCGGCGGCCCTGCTTAACCGCG
CACGTCGCACCGGCCGACGGGCGGCCATGCGAGCCGCTCGAAGGCTGGCCGCGGGTATTGTC
ACTGTGCCCCCCAGGTCCAGGCGACGAGCGGCCGCCGCAGCAGCCGCGGCCATTAGTGTTAT
GACTCAGGGTCGCAGGGGCAACGTGTACTGGGTGCGCGACTCGGTTAGCGGCCTGCGCGTGC
CCGTGCGCACCCGCCCCCCGCGCAACTAGATTGCAATAAAAAACTACTTAGACTCGTACTGT
TGTATGTATCCAGCGGCGGCGGCGCGCATCGAAGCTATGTCCAAGCGCAAAATCAAAGAAGA
GATGCTCCAGGTCATCGCGCCGGAGATCTATGGCCCCCCGAAGAAGGAAGAGCAGGATTACA
AGCCCCGAAAGCTAAAGCGGGTCAAAAAGAAAAAGAAAGATGATGATGATGATGAACTTGAC
GACGAGGTGGAACTGTTGCACGCGACCGCGCCCAGGCGACGGGTACAGTGGAAAGGTCGACG
CGTAAGACGTGTTTTGCGACCCGGCACCACCGTAGTCTTTACGCCCGGTGAGCGCTCCACCC
GCACCTACAAGCGCGTGTATGATGAGGTGTACGGCGACGAGGACCTGCTTGAGCAGGCCAAC
GAGCGCCTCGGGGAGTTTGCCTACGGAAAGCGGCATAAGGACATGCTGGCGTTGCCGCTGGA
CGAGGGCAACCCAACACCTAGCCTAAAGCCCGTGACACTGCAGCAGGTGCTGCCCGCGCTTG
CACCGTCCGAAGAAAAGCGCGGCCTAAAGCGCGAGTCTGGTGACTTGGCACCCACCGTGCAG
CTGATGGTACCCAAGCGTCAGCGACTGGAAGATGTCTTGGAAAAAATGACCGTGGAGCCTGG
GCTGGAGCCCGAGGTCCGCGTGCGGCCAATCAAGCAGGTGGCACCGGGACTGGGCGTGCAGA
CCGTGGACGTTCAGATACCCACCACCAGTAGCACTAGTATTGCCACTGCCACAGAGGGCATG
GAGACACAAACGTCCCCGGTTGCCTCGGCGGTGGCAGATGCCGCGGTGCAGGCGGCCGCTGC
GGCCGCGTCCAAGACCTCTACGGAGGTGCAAACGGACCCGTGGATGTTTCGTGTTTCAGCCC
CCCGGCGTCCGCGCCGTTCAAGGAAGTACGGCGCCGCCAGCGCGCTACTGCCCGAATATGCC
CTACATCCTTCCATCGCGCCTACCCCCGGCTATCGTGGCTACACCTACCGCCCCAGAAGACG
AGCAACTACCCGACGCCGAACCACCACTGGAACCCGCCGCCGCCGTCGCCGTCGCCAGCCCG
TGCTGGCCCCGATTTCCGTGCGCAGGGTGGCTCGCGAAGGAGGCAGGACCCTGGTGCTGCCA
ACAGCGCGCTACCACCCCAGCATCGTTTAAAAGCCGGTCTTTGTGGTTCTTGCAGATATGGC
CCTCACCTGCCGCCTCCGTTTCCCGGTGCCGGGATTCCGAGGAAGAATGCACCGTAGGAGGG
GCATGGCCGGCCACGGCCTGACGGGCGGCATGCGTCGTGCGCACCACCGGCGGCGGCGCGCG
TCGCACCGTCGCATGCGCGCCGGTATCCTGCCCCTCCTTATTCCACTGATCGCCGCGGCGAT
TGGCGCCGTGCCCGGAATTGCATCCGTGGCCTTGCAGGCGCAGAGACACTGATTAAAAACAA
GTTACATGTGGAAAAATCAAAATAAAAGTCTGGACTCTCACGCTCGCTTGGTCCTGTAACTA
TTTTGTAGAATGGAAGACATCAACTTTGCGTCACTGGCCCCGCGACACGGCTCGCGCCCGTT
CATGGGAAACTGGCAAGATATCGGCACCAGCAATATGAGCGGTGGCGCCTTCAGCTGGGGCT
CGCTGTGGAGCGGCATTAAAAATTTCGGTTCCGCCGTTAAGAACTATGGCAGCAAAGCCTGG
AACAGCAGCACAGGCCAGATGCTGAGGGACAAGTTGAAAGAGCAAAATTTCCAACAAAAGGT
GGTAGATGGCCTGGCCTCTGGCATTAGCGGGGTGGTGGACCTGGCCAACCAGGCAGTGCAAA
ATAAGATTAACAGTAAGCTTGATCCCCGCCCTCCCGTAGAGGAGCCTCCACCGGCCGTGGAG
ACAGTGTCTCCAGAGGGGCGTGGCGAAAAGCGTCCGCGACCCGACAGGGAAGAAACTCTGGT
GACGCAAATAGACGAGCCTCCCTCGTACGAGGAGGCACTAAAGCAAGGCCTGCCCACCACCC
GTCCCATCGCGCCCATGGCTACCGGAGTGCTGGGCCAGCACACACCCGTAACGCTGGACCTG
CCTCCCCCCGCCGACACCCAGCAGAAACCTGTGCTGCCAGGCCCGTCCGCCGTTGTTGTAAC
CCGTCCTAGCCGCGGGTCCCTGCGCCGCGCCGCCAGCGGTCCGCGATCGTTGCGGCCCGTAG
CCAGTGGCAACTGGCAAAGCACACTGAACAGCATCGTGGGTTTGGGGGTGCAATCCCTGAAG
CGCCGACGATGCTTCTGATAGCTAACGTGTCGTATGTGTGTCATGTATGCGTCCATGTCGCC
GCCAGAGGAGCTGCTGAGCCGCCGCGCGCCCGCTTTCCAAGATGGCTACCCCTTCGATGATG
CCGCAGTGGTCTTACATGCACATCTCGGGCCAGGACGCCTCGGAGTACCTGAGCCCCGGGCT
GGTGCAGTTCGCCCGCGCCACCGAGACGTACTTCAGCCTGAATAACAAGTTTAGAAACCCCA
CGGTGGCGCCTACGCACGACGTGACCACAGACCGGTCTCAGCGTTTGACGCTGCGGTTCATC
CCCGTGGACCGCGAGGATACTGCGTACTCGTACAAGGCGCGGTTCACCCTAGCTGTGGGTGA
TAACCGTGTGCTAGACATGGCTTCCACGTACTTTGACATCCGCGGCGTGCTGGACAGGGGCC
CTACTTTTAAGCCCTACTCTGGCACTGCCTACAACGCACTGGCCCCCAAGGGTGCCCCCAAC
TCGTGCGAGTGGGAACAAAATGAAACTGCACAAGTGGATGCTCAAGAACTTGACGAAGAGGA
GAATGAAGCCAATGAAGCTCAGGCGCGAGAACAGGAACAAGCTAAGAAAACCCATGTATATG
CCCAGGCTCCACTGTCCGGAATAAAAATAACTAAAGAAGGTCTACAAATAGGAACTGCCGAC
GCCACAGTAGCAGGTGCCGGCAAAGAAATTTTCGCAGACAAAACTTTTCAACCTGAACCACA
AGTAGGAGAATCTCAATGGAACGAAGCGGATGCCACAGCAGCTGGTGGAAGGGTTCTTAAAA
AGACAACTCCCATGAAACCCTGCTATGGCTCATACGCTAGACCCACCAATTCCAACGGCGGA
CAGGGCGTTATGGTTGAACAAAATGGTAAATTGGAAAGTCAAGTCGAAATGCAATTTTTTTC
CACATCCACAAATGCCACAAATGAAGTTAACAATATACAACCAACAGTTGTATTGTACAGCG
AAGATGTAAACATGGAAACTCCAGATACTCATCTTTCTTATAAACCTAAAATGGGGGATAAA
AATGCCAAAGTCATGCTTGGACAACAAGCAATGCCAAACAGACCAAATTACATTGCTTTTAG
AGACAATTTTATTGGTCTCATGTATTACAACAGCACAGGTAACATGGGTGTCCTTGCTGGTC
AGGCATCGCAGTTGAACGCTGTTGTAGATTTGCAAGACAGAAACACAGAGCTGTCCTACCAG
CTTTTGCTTGATTCAATTGGCGACAGAACAAGATACTTTTCAATGTGGAATCAAGCTGTTGA
CAGCTATGATCCAGATGTCAGAATTATTGAGAACCATGGAACTGAGGATGAGTTGCCAAATT
ATTGCTTTCCTCTTGGTGGAATTGGGATTACTGACACTTTTCAAGCTGTTAAAACAACTGCT
GCTAACGGGGACCAAGGCAATACTACCTGGCAAAAAGATTCAACATTTGCAGAACGCAATGA
AATAGGGGTGGGAAATAACTTTGCCATGGAAATTAACCTGAATGCCAACCTATGGAGAAATT
TCCTTTACTCCAATATTGCGCTGTACCTGCCAGACAAGCTAAAATACAACCCCACCAATGTG
GAAATATCTGACAACCCCAACACCTACGACTACATGAACAAGCGAGTGGTGGCTCCTGGGCT
TGTAGACTGCTACATTAACCTTGGGGCGCGCTGGTCTCTGGACTACATGGACAACGTTAATC
CCTTTAACCACCCCCGCCATGCGGGCCTGCGTTACCGCTCCATGTTGTTGGGAAACGGCCGC
TACGTGCCCTTTCACATTCAGGTGCCCCAAAAGTTTTTTGCCATTAAAAACCTCCTCCTCCT
GCCAGGCTCATACACATATGAATGGAACTTCAGGAAGGATGTTAACATGGTTCTGCAGAGCT
CTCTGGGAAACGACCTTAGAGTTGACGGGGCTAGCATTAAGTTTGACAGCATTTGTCTTTAC
GCCACCTTCTTCCCCATGGCCCACAACACGGCCTCCACGCTGGAAGCCATGCTCAGAAATGA
CACCAACGACCAGTCCTTTAATGACTACCTTTCCGCCGCCAACATGCTATATCCCATACCCG
CCAACGCCACCAACGTGCCCATCTCCATCCCATCGCGCAACTGGGCAGCATTTCGCGGTTGG
GCCTTCACACGCTTGAAGACAAAGGAAACCCCTTCCCTGGGATCAGGCTACGACCCTTACTA
CACCTACTCTGGCTCCATACCATACCTTGACGGAACCTTCTATCTTAATCACACCTTTAAGA
AGGTGGCCATTACTTTTGACTCTTCTGTTAGCTGGCCGGGCAACGACCGCCTGCTTACTCCC
AATGAGTTIGAGATTAAGCGCTCAGTTGACGGGGAGGGCTATAACGTAGCTCAGTGCAACAT
GACAAAGGACTGGTTCCTAGTGCAGATGTTGGCCAACTACAATATTGGCTACCAGGGCTTCT
ACATTCCAGAAAGCTACAAAGACCGCATGTACTCGTTCTTCAGAAACTTCCAGCCCATGAGC
CGGCAAGTGGTGGACGATACTAAATACAAAGATTATCAGCAGGTTGGAATTATCCACCAGCA
TAACAACTCAGGCTTCGTAGGCTACCTCGCTCCCACCATGCGCGAGGGACAAGCTTACCCCG
CTAATGTTCCCTACCCACTAATAGGCAAAACCGCGGTTGATAGTATTACCCAGAAAAAGTTT
CTTTGCGACCGCACCCTGTGGCGCATCCCCTTCTCCAGTAACTTTATGTCCATGGGTGCGCT
CACAGACCTGGGCCAAAACCTTCTCTACGCAAACTCCGCCCACGCGCTAGACATGACCTTTG
AGGTGGATCCCATGGACGAGCCCACCCTTCTTTATGTTTTGTTTGAAGTCTTTGACGTGGTC
CGTGTGCACCAGCCGCACCGCGGCGTCATCGAGACCGTGTACCTGCGCACGCCCTTCTCGGC
CGGCAACGCCACAACATAAAGAAGCAAGCAACATCAACAACAGCTGCCGCCATGGGCTCCAG
TGAGCAGGAACTGAAAGCCATTGTCAAAGATCTTGGTTGTGGGCCATATTTTTTGGGCACCT
ATGACAAGCGCTTCCCAGGCTTTGTTTCCCCACACAAGCTCGCCTGCGCCATAGTTAACACG
GCCGGTCGCGAGACTGGGGGCGTACACTGGATGGCCTTTGCCTGGAACCCGCGCTCAAAAAC
ATGCTACCTCTTTGAGCCCTTTGGCTTTTCTGACCAACGTCTCAAGCAGGTTTACCAGTTTG
AGTACGAGTCACTCCTGCGCCGTAGCGCCATTGCCTCTTCCCCCGACCGCTGTATAACGCTG
GAAAAGTCCACCCAAAGCGTGCAGGGGCCCAACTCGGCCGCCTGTGGCCTATTCTGCTGCAT
GTTTCTCCACGCCTTTGCCAACTGGCCCCAAACTCCCATGGATCACAACCCCACCATGAACC
TTATTACCGGGGTACCCAACTCCATGCTTAACAGTCCCCAGGTACAGCCCACCCTGCGCCGC
AACCAGGAACAGCTCTACAGCTTCCTGGAGCGCCACTCGCCCTACTTCCGCAGCCACAGTGC
GCAAATTAGGAGCGCCACTTCTTTTTGTCACTTGAAAAACATGTAAAAATAATGTACTAGGA
GACACTTTCAATAAAGGCAAATGTTTTTATTTGTACACTCTCGGGTGATTATTTACCCCCAC
CCTTGCCGTCTGCGCCGTTTAAAAATCAAAGGGGTTCTGCCGCGCATCGCTATGCGCCACTG
GCAGGGACACGTTGCGATACTGGTGTTTAGTGCTCCACTTAAACTCAGGCACAACCATCCGC
GGCAGCTCGGTGAAGTTTTCACTCCACAGGCTGCGCACCATCACCAACGCGTTTAGCAGGTC
GGGCGCCGATATCTTGAAGTCGCAGTTGGGGCCTCCGCCCTGCGCGCGCGAGTTGCGATACA
CAGGGTTACAGCACTGGAACACTATCAGCGCCGGGTGGTGCACGCTGGCCAGCACGCTCTTG
TCGGAGATCANATCCGCGTCCAGGTCCTCCGCGTTGCTCAGGGCGAACGGAGTCAACTTTGG
TAGCTGCCTTCCCAAAAAGGGTGCATGCCCAGGCTTTGAGTTGCACTCGCACCGTAGTGGCA
TCAGAAGGTGACCGTGCCCAGTCTGGGCGTTAGGATACAGCGCCTGCATGAAAGCCTTGATC
TGCTTAAAAGCCACCTGAGCCTTTGCGCCTTCAGAGAAGAACATGCCGCAAGACTTGCCGGA
AAACTGATTGGCCGGACAGGCCGCGTCATGCACGCAGCACCTTGCGTCGGTGTTGGAGATCT
GCACCACATTTCGGCCCCACCGGTTCTTCACGATCTTGGCCTTGCTAGACTGCTCCTTCAGC
GCGCGCTGCCCGTTTTCGCTCGTCACATCCATTTCAATCACGTGCTCCTTATTTATCATAAT
GCTCCCGTGTAGACACTTAAGCTCGCCTTCGATCTCAGCGCAGCGGTGCAGCCACAACGCGC
AGCCCGTGGGCTCGTGGTGCTTGTAGGTTACCTCTGCAAACGACTGCAGGTACGCCTGCAGG
AATCGCCCCATCATCGTCACAAAGGTCTTGTTGCTGGTGAAGGTCAGCTGCAACCCGCGGTG
CTCCTCGTTTAGCCAGGTCTTGCATACGGCCGCCAGAGCTTCCACTTGGTCAGGCAGTAGCT
TGAAGTTTGCCTTTAGATCGTTATCCACGTGGTACTTGTCCATCAACGCGCGCGCAGCCTCC
ATGCCCTTCTCCCACGCAGACACGATCGGCAGGCTCAGCGGGTTTATCACCGTGCTTTCACT
TTCCGCTTCACTGGACTCTTCCTTTTCCTCTTGCATCCGCATACCCCGCGCCACTGGGTCGT
CTTCATTCAGCCGCCGCACCGTGCGCTTACCTCCCTTGCCGTGCTTGATTAGCACCGGTGGG
TTGCTGAAACCCACCATTTGTAGCGCCACATCTTCTCTTTCTTCCTCGCTGTCCACGATCAC
CTCTGGGGATGGCGGGCGCTCGGGCTTGGGAGAGGGGCGCTTCTTTTTCTTTTTGGACGCAA
TGGCCAAATCCGCCGTCGAGGTCGATGGCCGCGGGCTGGGTGTGCGCGGCACCAGCGCATCT
TGTGACGAGTCTTCTTCGTCCTCGGACTCGAGACGCCGCCTCAGCCGCTTTTTTGGGGGCGC
GCGGGGAGGCGGCGGCGACGGCGACGGGGACGAGACGTCCTCCATGGTTGGTGGACGTCGCG
CCGCACCGCGTCCGCGCTCGGGGGTGGTTTCGCGCTGCTCCTCTTCCCGACTGGCCATTTCC
TTCTCCTATAGGCAGAAAAAGATCATGGAGTCAGTCGAGAAGGAGGACAGCCTAACCGCCCC
CTTTGAGTTCGCCACCACCGCCTCCACCGATGCCGCCAACGCGCCTACCACCTTCCCCGTCG
AGGCACCCCCGCTTGAGGAGGAGGAAGTGATTATCGAGCAGGACCCAGGTTTTGTAAGCGAA
GACGACGAAGATCGCTCAGTACCAACAGAGGATAAAAAGCAAGACCAGGACGACGCAGAGGC
AAACGAGGAACAAGTCGGGCGGGGGGACCAAAGGCATGGCGACTACCTAGATGTGGGAGACG
ACGTGCTGTTGAAGCATCTGCAGCGCCAGTGCGCCATTATCTGCGACGCGTTGCAAGAGCGC
AGCGATGTGCCCCTCGCCATAGCGGATGTCAGCCTTGCCTACGAACGCCACCTGTTCTCACC
GCGCGTACCCCCCAAACGCCAAGAAAACGGCACATGCGAGCCCAACCCGCGCCTCAACTTCT
ACCCCGTATTTGCCGTGCCAGAGGTGCTTGCCACCTATCACATCTTTTTCCAAAACTGCAAG
ATACCCCTATCCTGCCGTGCCAACCGCAGCCGAGCGGACAAGCAGCTGGCCTTGCGGCAGGG
CGCTGTCATACCTGATATCGCCTCGCTCGACGAAGTGCCAAAAATCTTTGAGGGTCTTGGAC
GCGACGAGAAGCGCGCGGCAAACGCTCTGCAACAAGAAAACAGCGAAAATGAAAGTCACTGT
GGAGTGCTGGTGGAACTTGAGGGTGACAACGCGCGCCTAGCCGTGCTGAAACGCAGCATCGA
GGTCACCCACTTTGCCTACCCGGCACTTAACCTACCCCCCAAGGTTATGAGCACAGTCATGA
GCGAGCTGATCGTGCGCCGTGCACGACCCCTGGAGAGGGATGCAAACTTGCAAGAACAAACC
GAGGAGGGCCTACCCGCAGTTGGCGATGAGCAGCTGGCGCGCTGGCTTGAGACGCGCGAGCC
TGCCGACTTGGAGGAGCGACGCAAGCTAATGATGGCCGCAGTGCTTGTTACCGTGGAGCTTG
AGTGCATGCAGCGGTTCTTTGCTGACCCGGAGATGCAGCGCAAGCTAGAGGAAACGTTGCAC
TACACCTTTCGCCAGGGCTACGTGCGCCAGGCCTGCAAAATTTCCAACGTGGAGCTCTGCAA
CCTGGTCTCCTACCTTGGAATTTTGCACGAAAACCGCCTTGGGCAAAACGTGCTTCATTCCA
CGCTCAAGGGCGAGGCGCGCCGCGACTACGTCCGCGACTGCGTTTACTTATTTCTGTGCTAC
ACCTGGCAAACGGCCATGGGCGTGTGGCAGCAGTGCCTGGAGGAGCGCAACCTGAAGGAGCT
GCAGAAGCTGCTAAAGCAAAACTTGAAGGACCTATGGACGGCCTTCAACGAGCGCTCCGTGG
CCGCGCACCTGGCGGACATTATCTTCCCCGAACGCCTGCTTAAAACCCTGCAACAGGGTCTG
CCAGACTTCACCAGTCAAAGCATGTTGCAAAACTTTAGGAACTTTATCCTAGAGCGTTCAGG
AATTCTGCCCGCCACCTGCTGTGCGCTTCCTAGCGACTTTGTGCCCATTAAGTACCGTGAAT
GCCCTCCGCCGCTTTGGGGTCACTGCTACCTTNTGCAGCTAGCCAACTACCTTGCCTACCAC
TCCGACATCATGGAAGACGTGAGCGGTGACGGCCTACTGGAGTGTCACTGTCGCTGCAACCT
ATGCACCCCGCACCGCTCCCTGGTCTGCAATTCACAACTGCTTAGCGAAAGTCAAATTATCG
GTACCTTTGAGCTGCAGGGTCCCTCGCCTGACGAAAAGTCCGCGGCTCCGGGGTTGAAACTC
ACTCCGGGGCTGTGGACGTCGGCTTACCTTCGCAAATTTGTACCTGAGGACTACCACGCCCA
CGAGATTAGGTTCTACGAAGACCAATCCCGCCCGCCAAATGCGGAGCTTACCGCCTGCGTCA
TTACCCAGGGCCACATCCTTGGCCAATTGCAAGCCATTAACAAAGCCCGCCAAGAGTTTCTG
CTACGAAAGGGACGGGGGGTTTACTTGGACCCCCAGTCCGGCGAGGAGCTCAACCCAATCCC
CCCGCCGCCGCAGCCCTATCAGCAGCCGCGGGCCCTTGCTTCCCAGGATGGCACCCAAAAAG
AAGCTGCAGCTGCCGCCGCCGCCACCCACGGACGAGGAGGAATACTGGGACAGTCAGGCAGA
GGAGGTTTTGGACGAGGAGGAGGAGATGATGGAAGACTGGGACAGCCTAGACGAGGAAGCTT
CCGAGGCCGAAGAGGTGTCAGACGAAACACCGTCACCCTCGGTCGCATTCCCCTCGCCGGCG
CCCCAGAAATCGGCAACCGTTCCCAGCATTGCTACAACCTCCGCTCCTCAGGCGCCGCCGGC
ACTGCCCGTTCGCCGACCCAACCGTAGATGGGACACCACTGGAACCAGGGCCGGTAAGTCTA
AGCAGCCGCCGCCGTTAGCCCAAGAGCAACAACAGCGCCAAGGCTACCGCTCGTGGCGCGTG
CACAAGAACGCCATAGTTGCTTGCTTGCAAGACTGTGGGGGCAACATCTCCTTCGCCCGCCG
CTTTCTTCTCTACCATCACGGCGTGGCCTTCCCCCGTAACATCCTGCATTACTACCGTCATC
TCTACAGCCCCTACTGCACCGGCGGCAGCGGCAGCAACAGCAGCGGCCACGCAGAAGCAAAG
GCGACCGGATAGCAAGACTCTGACAAAGCCCAAGAAATCCACAGCGGCGGCAGCAGCAGGAG
GAGGAGCACTGCGTCTGGCGCCCAACGAACCCGTATCGACCCGCGAGCTTAGAAACAGGATT
TTTCCCACTCTGTATGCTATATTTCAACAGAGCAGGGGCCAAGAACAAGAGCTGAAAATAAA
AAACAGGTCTCTGCGCTCCCTCACCCGCAGCTGCCTGTATCACAAAAGCGAAGATCAGCTTC
GGCGCACGCTGGAAGACGCGGAGGCTCTCTTCAGCAAATACTGCGCGCTGACTCTTAAGGAC
TAGTTTCGCGCCCTTTCTCAAATTTAAGCGCGAAAACTACGTCATCTCCAGCGGCCACACCC
GGCGCCAGCACCTGTCGTCAGCGCCATTATGAGCAAGGAAATTCCCACGCCCTACATGTGGA
GTTACCAGCCACAAATGGGACTTGCGGCTGGAGCTGCCCAAGACTACTCAACCCGAATAAAC
TACATGAGCGCGGGACCCCACATGATATCCCGGGTCAACGGAATCCGCGCCCACCGAAACCG
AATTCTCCTCGAACAGGCGGCTATTACCACCACACCTCGTAATAACCTTAATCCCCGTAGTT
GGCCCGCTGCCCTGGTGTACCAGGAAAGTCCCGCTCCCACCACTGTGGTACTTCCCAGAGAC
GCCCAGGCCGAAGTTCAGATGACTAACTCAGGGGCGCAGCTTGCGGGCGGCTTTCGTCACAG
GGTGCGGTCGCCCGGGCAGGGTATAACTCACCTGAAAATCAGAGGGCGAGGTATTCAGCTCA
ACGACGAGTCGGTGAGCTCCTCTCTTGGTCTCCGTCCGGACGGGACATTTCAGATCGGCGGC
GCTGGCCGCTCTTCATTTACGCCCCGTCAGGCGATCCTAACTCTGCAGACCTCGTCCTCGGA
GCCGCGCTCCGGAGGCATTGGAACTCTACAATTTATTGAGGAGTTCGTGCCTTCGGTTTACT
TCAACCCCTTTTCTGGACCTCCCGGCCACTACCCGGACCAGTTTATTCCCAACTTTGACGCG
GTAAAAGACTCGGCGGACGGCTACGACTGACAGATCTGAGCTCGCGGCCGCGATATCGCTAG
CGAAGTTCCTATTCTCTAGAAAGTATAGGAACTTCGATCCTCTAGAGTCGACCTGCAGGCAT
GCAAGCTTGGCACTGCAATAAATTACTTACTTAAAATCAGTCAGCAAATCTTTGTCCAGCTT
ATTCAGCATCACCTCCTTTCCCTCCTCCCAACTCTGGTATTTCAGCAGCCTTTTAGCTGCGA
ACTTTCTCCAAAGTCTAAATGGGATGTCAAATTCCTCATGTTCTTGTCCCTCCGCACCCACT
ATCTTCATATTGTTGCAGATGAAACGCGCCAGACCGTCTGAAGACACCTTCAACCCTGTGTA
CCCATATGACACGGAAACCGGCCCTCCAACTGTGCCTTTCCTTACCCCTCCCTTTGTGTCGC
CAAATGGGTTCCAAGAAAGTCCCCCCGGAGTGCTTTCTTTGCGTCTTTCAGAACCTTTGGTT
ACCTCACACGGCATGCTTGCGCTAAAAATGGGCAGCGGCCTGTCCCTGGATCAGGCAGGCAA
CCTTACATCAAATACAATCACTGTTTCTCAACCGCTAAAAAAAACAAAGTCCAATATAACTT
TGGAAACATCCGCGCCCCTTACAGTCAGCTCAGGCGCCCTAACCATGGCCACAACTTCGCCT
TTGGTGGTCTCTGACAACACTCTTACCATGCAATCACAAGCACCGCTAACCGTGCAAGACTC
AAAACTTAGCATTGCTACCAAAGAGCCACTTACAGTGTTAGATGGAAAACTGGCCCTGCAGA
CATCAGCCCCCCTCTCTGCCACTGATAACAACGCCCTCACTATCACTGCCTCACCTCCTCTT
ACTACTGCAAATGGTAGTCTGGCTGTTACCATGGAAAACCCACTTTACAACAACAATGGAAA
ACTTGGGCTCAAAATTGGCGGTCCTTTGCAAGTGGCCACCGACTCACATGCACTAACACTAG
GTACTGGTCAGGGGGTTGCAGTTCATAACAATTTGCTACATACAAAAGTTACAGGCGCAATA
GGGTTTGATACATCTGGCAACATGGAACTTAAAACTGGAGATGGCCTCTATGTGGATAGCGC
CGGTCCTAACCAAAAACTACATATTAATCTAAATACCACAAAAGGCCTTGCTTTTGACAACA
CCGCAATAACAATTAACGCTGGAAAAGGGTTGGAATTTGAAACAGACTCCTCAAACGGAAAT
CCCATAAAAACAAAAATTGGATCAGGCATACAATATAATACCAATGGAGCTATGGTTGCAAA
ACTTGGAACAGGCCTCAGTTTTGACAGCTCCGGAGCCATAACAATGGGCAGCATAAACAATG
ACAGACTTACTCTTTGGACAACACCAGACCCATCCCCAAATTGCAGAATTGCTTCAGATAAA
GACTGCAAGCTAACTCTGGCGCTAACAAAATGTGGCAGTCAAATTTTGGGCACTGTTTCAGC
TTTGGCAGTATCAGGTAATATGGCCTCCATCAATGGAACTCTAAGCAGTGTAAACTTGGTTC
TTAGATTTGATGACAACGGAGTGCTTATGTCAAATTCATCACTGGACAAACAGTATTGGAAC
TTTAGAAACGGGGACTCCACTAACGGTCAACCATACACTTATGCTGTTGGGTTTATGCCAAA
CCTAAAAGCTTACCCAAAAACTCAAAGTAAAACTGCAAAAAGTAATATTGTTAGCCAGGTGT
ATCTTAATGGTGACAAGTCTAAACCATTGCATTTTACTATTACGCTAAATGGAACAGATGAA
ACCAACCAAGTAAGCAAATACTCAATATCATTCAGTTGGTCCTGGAACAGTGGACAATACAC
TAATGACAAATTTGCCACCAATTCCTATACCTTCTCCTACATTGCCCAGGAATAAAGAATCG
TGAACCTGTTGCATGTTATGTTTCAACGTGTTTATTTTTCAATTCGTATTAGTCATCGCTAT
TACCATGGTGATGCGGTTTTGGCAGTACATCAATGGGCGTGGATAGCGGTTTGACTCACGGG
GATTTCCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCGTGTACG
GTGGGAGGTCTATATAAGCAGAGCTGGTTTAGTGAACCGTCAGATCCGCTAGAGATCCACCA
TGTTTGTCTTTCTCGTGCTGCTGCCCCTCGTGAGCAGCCAGTGCGTCAATCTGACAACAAGG
ACCCAGCTGCCCCCCGCCTACACCAACTCCTTCACAAGAGGCGTGTATTACCCCGATAAGGT
CTTCAGATCCAGCGTCCTCCACAGCACCCAAGATTTGTTTCTGCCTTTCTTCAGCAACGTGA
CATGGTTCCACGCCATTCATGTCAGCGGCACAAACGGCACAAAGAGGTTTGACAACCCCGTG
CTCCCCTTCAACGACGGCGTGTACTTCGCCAGCACAGAGAAATCCAATATCATTAGGGGCTG
GATCTTCGGCACAACACTGGATTCCAAGACCCAGTCTCTGCTCATTGTGAATAACGCCACCA
ACGTGGTGATTAAGGTCTGTGAGTTTCAGTTCTGCAACGACCCCTTTCTGGGAGTCTACTAC
CACAAGAATAATAAGAGCTGGATGGAGTCCGAGTTTAGGGTGTACAGCTCCGCCAACAACTG
TACCTTCGAATACGTGTCCCAGCCTTTCCTCATGGATCTGGAGGGCAAGCAAGGCAATTTCA
AAAATCTGAGAGAGTTCGTGTTCAAAAACATTGATGGATACTTCAAAATCTACAGCAAGCAT
ACCCCCATTAATCTGGTGAGGGATCTGCCCCAAGGATTCTCCGCTCTGGAACCTCTGGTGGA
TCTGCCCATTGGCATTAACATCACAAGATTCCAGACCCTCCTCGCCCTCCATAGATCCTATC
TGACCCCCGGCGACTCCTCCAGCGGATGGACAGCCGGAGCTGCCGCCTACTACGTGGGCTAT
CTGCAGCCAAGAACCTTTCTGCTGAAGTACAACGAGAACGGCACCATCACAGACGCTGTCGA
TTGCGCTCTCGACCCTCTGAGCGAGACCAAATGCACACTGAAGAGCTTCACCGTGGAAAAGG
GCATCTATCAGACCAGCAACTTCAGAGTGCAGCCTACCGAGAGCATTGTGAGGTTTCCCAAC
ATCACCAATCTGTGTCCTTTCGGCGAGGTCTTTAATGCCACAAGGTTCGCTTCCGTGTATGC
TTGGAATAGGAAGAGGATCAGCAATTGCGTCGCCGACTATTCCGTCCTCTATAACAGCGCCT
CCTTCTCCACCTTCAAATGTTATGGCGTGTCCCCCACCAAGCTCAACGACCTCTGCTTCACC
AATGTGTACGCTGACTCCTTCGTCATTAGGGGCGACGAGGTGAGGCAAATTGCCCCCGGCCA
GACCGGCAAGATTGCTGATTACAACTACAAACTGCCCGACGATTTTACCGGCTGCGTGATCG
CTTGGAACTCCAACAATCTGGACTCCAAAGTGGGCGGAAACTACAATTACCTCTACAGACTC
TTTAGAAAAAGCAATCTGAAGCCCTTCGAGAGAGACATCTCCACCGAAATCTACCAAGCCGG
AAGCACACCTTGCAATGGCGTCGAGGGATTTAACTGCTACTTCCCTCTGCAGAGCTACGGCT
TTCAACCTACCAACGGCGTCGGATATCAACCCTATAGGGTGGTCGTGCTGAGCTTTGAACTG
CTGCATGCTCCCGCCACCGTCTGCGGACCTAAGAAGAGCACCAATCTCGTCAAAAACAAGTG
CGTGAACTTCAACTTCAATGGACTGACCGGCACCGGCGTGCTGACCGAGAGCAATAAGAAGT
TTCTGCCCTTCCAGCAGTTCGGAAGGGATATTGCCGATACCACAGATGCTGTGAGGGACCCC
CAAACCCTCGAGATTCTGGATATCACCCCTTGCAGCTTCGGAGGAGTGTCCGTGATCACCCC
CGGAACAAACACCTCCAATCAAGTGGCTGTGCTGTACCAAGACGTGAACTGCACAGAAGTCC
CCGTGGCCATCCATGCCGACCAGCTGACCCCTACATGGAGAGTGTACTCCACCGGCAGCAAT
GTGTTCCAGACAAGAGCCGGATGCCTCATTGGAGCTGAACACGTCAACAACAGCTACGAGTG
CGACATTCCCATCGGCGCCGGCATTTGTGCCTCCTATCAGACCCAGACCAACAGCCCAAGAA
GGGCTAGAAGCGTCGCTTCCCAATCCATCATTGCCTACACCATGTCTCTGGGAGCCGAAAAC
TCCGTCGCCTACTCCAACAATAGCATCGCCATCCCCACCAATTTTACCATCTCCGTGACCAC
AGAGATTCTGCCCGTGTCCATGACAAAGACATCCGTGGACTGCACCATGTACATCTGTGGCG
ACAGCACCGAGTGTAGCAATCTGCTGCTGCAATATGGCAGCTTCTGCACCCAGCTGAACAGA
GCCCTCACCGGCATCGCCGTCGAACAAGACAAGAACACCCAAGAGGTGTTCGCCCAAGTGAA
GCAAATCTACAAGACCCCCCCTATCAAAGATTTCGGAGGATTCAACTTTAGCCAGATTCTGC
CCGATCCTAGCAAGCCTTCCAAGAGGAGCTTCATCGAGGATCTGCTGTTTAATAAGGTGACA
CTGGCCGACGCTGGCTTCATTAAACAGTACGGCGATTGTCTGGGCGACATCGCTGCTAGGGA
TCTGATCTGCGCTCAGAAGTTCAACGGACTGACAGTCCTCCCTCCTCTGCTGACCGACGAGA
TGATCGCTCAGTATACCAGCGCTCTGCTGGCTGGAACCATTACCAGCGGCTGGACATTCGGC
GCTGGAGCCGCCCTCCAAATTCCCTTTGCCATGCAGATGGCCTATAGATTCAACGGCATTGG
CGTCACCCAAAATGTGCTGTATGAAAATCAGAAGCTGATTGCTAACCAATTCAATAGCGCCA
TTGGCAAGATCCAAGACTCTCTGAGCTCCACAGCCAGCGCCCTCGGAAAGCTGCAAGACGTG
GTGAATCAAAACGCCCAAGCTCTGAACACACTGGTGAAACAGCTCAGCAGCAACTTTGGAGC
CATCAGCAGCGTGCTCAATGATATCCTCTCTAGGCTGGACAAAGTGGAGGCCGAAGTCCAGA
TCGATAGACTCATCACCGGCAGACTCCAATCTCTGCAGACATACGTCACCCAACAGCTCATT
AGAGCTGCCGAAATCAGAGCCTCCGCCAATCTGGCCGCCACCAAGATGTCCGAGTGCGTGCT
GGGACAGAGCAAGAGAGTGGACTTCTGTGGCAAGGGATACCATCTGATGAGCTTCCCCCAGA
GCGCTCCCCATGGAGTGGTCTTTCTGCATGTCACATACGTGCCCGCCCAAGAGAAGAACTTC
ACCACCGCTCCCGCCATTTGCCACGATGGAAAGGCCCACTTTCCCAGAGAAGGAGTGTTCGT
GAGCAACGGCACACACTGGTTTGTCACCCAGAGAAATTTTTACGAGCCCCAGATTATCACCA
CCGACAACACCTTCGTGTCCGGAAACTGCGATGTCGTGATTGGCATCGTGAACAACACAGTC
TACGACCCTCTGCAGCCCGAACTCGACAGCTTCAAGGAAGAGCTGGACAAGTACTTCAAGAA
TCACACATCCCCCGACGTGGATCTGGGCGACATTAGCGGCATTAATGCCTCCGTCGTCAACA
TTCAGAAGGAGATTGATAGACTGAATGAAGTCGCCAAGAACCTCAATGAGTCTCTGATTGAT
CTGCAAGAGCTGGGCAAGTACGAGCAATACATCAAATGGCCTTGGTACATCTGGCTGGGATT
CATCGCTGGACTCATCGCCATCGTGATGGTCACCATTATGCTGTGTTGCATGACCAGCTGCT
GCAGCTGTCTGAAGGGCTGCTGCAGCTGCGGAAGCTGCTGCAAGTTTGACGAAGACGACTCC
GAGCCCGTGCTGAAGGGCGTCAAGCTGCATTATACATAAACTAGTGCTGGAATTCGCCCTTA
TAGAGTGCTGGAATTCGCCCTTATAGAGTGCTGGAATTCGCCCTTATATCTAGTAACGGCCG
CCAGTGTGCTGGAATTCGCCCTTATAACTTCGTATAGCATACATTATACGAAGTTATTGTTG
ACAATTAATCATCGGCATAGTATATCGGCATAGTATAATACGACAAGGTGAGGAACTAAACC
ATGGCCAAGTTGACCAGTGCCGTTCCGGTGCTCACCGCGCGCGACGTCGCCGGAGCGGTCGA
GTTCTGGACCGACCGGCTCGGGTTCTCCCGGGACTTCGTGGAGGACGACTTCGCCGGTGTGG
TCCGGGACGACGTGACCCTGTTCATCAGCGCGGTCCAGGACCAGGTGGTGCCGGACAACACC
CTGGCCTGGGTGTGGGTGCGCGGCCTGGACGAGCTGTACGCCGAGTGGTCGGAGGTCGTGTC
CACGAACTTCCGGGACGCCTCCGGGCCGGCCATGACCGAGATCGGCGAGCAGCCGTGGGGGC
GGGAGTTCGCCCTGCGCGACCCGGCCGGCAACTGCGTGCACTTCGTGGCCGAGGAGCAGGAC
TGAATAACTTCGTATAGCATACATTATACGAAGTTATAAGGGCGAATTCTGCAGATATCCAT
CCTTTAAAAAACCTCCCACACCTCCCCCTGAACCTGAAACATAAAATGAATGCAATTGTTGT
TGTTAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCT
TAACAACGTGTTTATTTTTCAATTGCAGAAAGAATTGCAGAAAATTTCAAGTCATTTTTCAT
TCAGTAGTATAGCCCCACCACCACATAGCTTATACTAATCACCGTACCTTAATCAAACTCAC
AGAACCCTAGTATTCAACCTGCCACCTCCCTCCCAACACACAGAGTACACAGTCCTTTCTCC
CCGGCTGGCCTTAAACAGCATCATATCATGGGTAACAGACATATTCTTAGGTGTTATATTCC
ACACGGTCTCCTGTCGAGCCAAACGCTCATCAGTGATGTTAATAAACTCCCCGGGCAGCTCG
CTTAAGTTCATGTCGCTGTCCAGCTGCTGAGCCACAGGCTGCTGTCCAACTTGCGGTTGCTC
AACGGGCGGCGAAGGAGAAGTCCACGCCTACATGGGGGTAGAGTCATAATCGTGCATCAGGA
TAGGGCGGTGGTGCTGCAGCAGCGCGCGAATAAACTGCTGCCGCCGCCGCTCCGTCCTGCAG
GAATACAACATGGCAGTGGTCTCCTCAGCGATGATTCGCACCGCCCGCAGCATAAGGCGCCT
TGTCCTCCGGGCACAGCAGCGCACCCTGATCTCACTTAAGTCAGCACAGTAACTGCAGCACA
GTACCACAATATTGTTTAAAATCCCACAGTGCAAGGCGCTGTATCCAAAGCTCATGGCGGGG
ACCACAGAACCCACGTGGCCATCATACCACAAGCGCAGGTAGATTAAGTGGCGACCCCTCAT
AAACACGCTGGACATAAACATTACCTCTTTTGGCATGTTGTAATTCACCACCTCCCGGTACC
ATATAAACCTCTGATTAAACATGGCGCCATCCACCACCATCCTAAACCAGCTGGCCAAAACC
TGCCCGCCGGCTATGCACTGCAGGGAACCGGGACTGGAACAATGACAGTGGAGAGCCCAGGA
CTCGTAACCATGGATCATCATGCTCGTCATGATATCAATGTTGGCACAACACAGGCACACGT
GCATACACTTCCTCAGGATTACAAGCTCCTCCCGCGTCAGAACCATATCCCAGGGAACAACC
CATTCCTGAATCAGCGTAAATCCCACACTGCAGGGAAGACCTCGCACGTAACTCACGTTGTG
CATTGTCAAAGTGTTACATTCGGGCAGCAGCGGATGATCCTCCAGTATGGTAGCGCGTGTCT
CTGTCTCAAAAGGAGGTAGGCGATCCCTACTGTACGGAGTGCGCCGAGACAACCGAGATCGT
GTTGGTCGTAGTGTCATGCCAAATGGAACGCCGGACGTAGTCATATTTCCTGAAGCAAAACC
AGGTGCGGGCGTGACAAACAGATCTGCGTCTCCGGTCTCGTCGCTTAGCTCGCTCTGTGTAG
TAGTTGTAGTATATCCACTCTCTCAAAGCATCCAGGCGCCCCCTGGCTTCGGGTTCTATGTA
AACTCCTTCATGCGCCGCTGCCCTGATAACATCCACCACCGCAGAATAAGCCACACCCAGCC
AACCTACACATTCGTTCTGCGAGTCACACACGGGAGGAGCGGGAAGAGCTGGAAGAACCATG
TTTTTTTTTTTTATTCCAAAAGATTATCCAAAACCTCAAAATGAAGATCTATTAAGTGAACG
CGCTCCCCTCCGGTGGCGTGGTCAAACTCTACAGCCAAAGAACAGATAATGGCATTTGTAAG
ATGTTGCACAATGGCTTCCAAAAGGCAAACTGCCCTCACGTCCAAGTGGACGTAAAGGCTAA
ACCCTTCANGGTGAATCTCCTCTATAAACATTCCAGCACCTTCAACCATGCCCAAATAATTT
TCATCTCGCCACCTTATCAATATGTCTCTAAGCAAATCCCGAATATTAAGTCCGGCCATTGT
AAAAATCTGCTCCAGAGCGCCCTCCACCTTCAGCCTCAAGCAGCGAATCATGATTGCAAAAA
TTCAGGTTCCTCACAGACCTGTATAAGATTCAAAAGCGGAACATTAACAAAAATACCGCGAT
CCCGTAGGTCCCTTCGCAGGGCCAGCTGAACATAATCGTGCAGGTCTGCACGGACCAGCGCG
GCCACTTCCCCGCCAGGAACCATGACAAAAGAACCCACACTGATTATGACACGCATACTCGG
AGCTATGCTAACCAGCGTAGCCCCGATGTAAGCTTGTTGCATGGGCGGCGATATAAAATGCA
AGGTACTGCTCAAAAAATCAGGCAAAGCCTCGCGCAAAAAAGCAAGCACATCGTAGTCATGC
TCATGCAGATAAAGGCAGGTAAGTTCCGGAACCACCACAGAAAAAGACACCATTTTTCTCTC
AAACATGTCTGCGGGTTCCTGCATAAACACAAAATAAAATAACAAAAAAAAAAAAACATTTA
AACATTAGAAGCCTGTCTTACAACAGGAAAAACAACCCTTATAAGCATAAGACGGACTACGG
CCATGCCGGCGTGACCGTAAAAAAACTGGTCACCGTGATTAAAAAGCACCACCGACAGTTCC
TCGGTCATGTCCGGAGTCATAATGTAAGACTCGGTAAACACATCAGGTTGGTTAACATCGGT
CAGTGCTAAAAAGCGACCGAAATAGCCCGGGGGAATACATACCCGCAGGCGTAGAGACAACA
TTACAGCCCCCATAGGAGGTATAACAAAATTAATAGGAGAGAAAAACACATAAACCCCTGAA
AAACCCTCCTGCCCCTAGGCAAAATAGCACCCTCCCGCTCCAGAACAACATACAGCGCTTCC
ACAGCGGCAGCCATAACAGTCAGCCTTACCAGTAAAAAAACCTATTAAAAAACACCACTCGA
CACGGCACCAGCTCAATCAGTCACAGTGTAAAAAGGGCCAAGTACAGAGCGAGTATATATAG
GACTAAAAAATGACGTAACGGTTAAAGTCCACAAAAACCACCCAGAAAACCGCACGCGAACC
TACGCCCAGAAACGAAAGCCAAAAAACCCACAACTTCCTCAAATCTTCACTTCCGTTTTCCC
ACGATACGTCACTTCCCATTTTAAAAAAAAACTACAATTCCCAATACATGCAAGTTACTCCG
CCCTAAAACCTACGTCACCCGCCCCGTTCCCACGCCCCGCGCCACGTCACAAACTCCACCCC
CTCATTATCATATTGGCTTCAATCCAAAATAAGGTATATTATTGATGATGGCGAT

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for providing a human who received a prior coronavirus vaccine with a heterologous vaccination boost, wherein said method comprises intranasally administering a single-cycle adenovirus (SC-Ad) to said human, wherein said SC-Ad comprises a genome lacking at least a portion of a nucleic acid sequence that encodes an adenovirus polypeptide, wherein said SC-Ad comprises said adenovirus polypeptide, wherein said SC-Ad comprises a nucleic acid sequence encoding an immunogen of a coronavirus, and wherein said prior coronavirus vaccine did not comprise said SC-Ad.

2. The method of claim 1, wherein said adenovirus polypeptide is selected from the group consisting of a fiber polypeptide, a V polypeptide, a hexon polypeptide, a penton base polypeptide, and a pIIIa polypeptide.

3. The method of any one of claims 1-2, wherein said immunogen comprises a coronavirus Spike polypeptide or an immunogenic fragment thereof.

4. The method of claim 3, wherein said immunogen consists of or consists essentially of an amino acid sequence set forth in any one of SEQ ID NOs:1-4 of International PCT Patent Publication No. WO 2022/040204.

5. The method of any one of claims 1-4, wherein said SC-Ad further comprises a nucleic acid sequence encoding an adjuvant polypeptide.

6. The method of claim 5, wherein said adjuvant polypeptide is selected from the group consisting of a granulocyte-macrophage colony-stimulating factor (GM-CSF) polypeptide, an interleukin 4 (IL-4) polypeptide, an interleukin 21 (IL-21) polypeptide, a CD40 ligand (CD40L) polypeptide, a 4-1BB ligand (4-1BBL) polypeptide, a transforming growth factor beta (TGF-β) polypeptide, a Clostridium difficile TcdA polypeptide, a C. difficile TcdB polypeptide, and biologically active fragments thereof.

7. The method of claim 5 or claim 6, wherein said coronavirus Spike polypeptide is fused to said adjuvant polypeptide.

8. The method of claim 7, wherein said coronavirus Spike polypeptide fused to said adjuvant polypeptide consists essentially of or consists of an amino acid sequence set forth in SEQ ID NO:5 of International PCT Patent Publication No. WO 2022/040204.

9. The method of any one of claims 1-4, wherein said SC-Ad further comprises a nucleic acid sequence encoding a chaff polypeptide.

10. The method of claim 9, wherein said chaff polypeptide is a fragment of an ACE2 polypeptide.

11. The method of claim 10, wherein said fragment of an ACE2 polypeptide comprises the extracellular region of an ACE2 polypeptide and lacks a transmembrane domain.

12. The method of claim 11, wherein said chaff polypeptide consists essentially of or consists of an amino acid sequence set forth in SEQ ID NO:8 of International PCT Patent Publication No. WO 2022/040204 or SEQ ID NO:9 of International PCT Patent Publication No. WO 2022/040204.

13. The method of any one of claims 9-12, wherein said coronavirus Spike polypeptide is fused to said chaff polypeptide.

14. The method of any one of claims 1-13, wherein said coronavirus is a betacoronavirus.

15. The method of claim 14, wherein said betacoronavirus is SARS-CoV-2.

16. The method of any one of claims 1-15, wherein said human is a human who received said prior coronavirus vaccine at least 90 days before said intranasally administering.

17. The method of any one of claims 1-15, wherein said human is a human who received said prior coronavirus vaccine at least 120 days before said intranasally administering.

18. The method of any one of claims 1-15, wherein said human is a human who received said prior coronavirus vaccine at least 150 days before said intranasally administering.

19. The method of any one of claims 1-15, wherein said human is a human who received said prior coronavirus vaccine at least 180 days before said intranasally administering.

20. The method of any one of claims 1-19, wherein said prior coronavirus vaccine was an mRNA-based vaccine.

21. The method of any one of claims 1-20, wherein said SC-Ad infects a cell of said human, and wherein expression of said immunogen in said cell leads to an increase in the amount of serum IgG antibodies that bind to said immunogen.

22. The method of any one of claims 1-21, wherein said SC-Ad infects a cell of said human, and wherein expression of said immunogen in said cell leads to an increase in the amount of mucosal IgA antibodies that bind to said immunogen.

23. The method of any one of claims 1-22, wherein said SC-Ad infects a cell of said human, and wherein expression of said immunogen in said cell leads to an increase in the amount of neutralizing antibodies in said human that bind to said immunogen.

24. The method of any one of claims 1-23, wherein said SC-Ad infects a cell of said human, and wherein expression of said immunogen in said cell leads to an increase in the number of Th1 T cells within said human that target said immunogen.

25. A method for providing a mammal who received a prior vaccine targeting a pathogen with a heterologous vaccination boost against said pathogen, wherein said method comprises intranasally administering a single-cycle adenovirus (SC-Ad) to said mammal, wherein said SC-Ad comprises a genome lacking at least a portion of a nucleic acid sequence that encodes an adenovirus polypeptide, wherein said SC-Ad comprises said adenovirus polypeptide, wherein said SC-Ad comprises a nucleic acid sequence encoding an immunogen of said pathogen, and wherein said prior vaccine did not comprise said SC-Ad.

26. The method of claim 25, wherein said mammal is a human.

27. The method of any one of claims 25-26, wherein said adenovirus polypeptide is selected from the group consisting of a fiber polypeptide, a V polypeptide, a hexon polypeptide, a penton base polypeptide, and a pIIIa polypeptide.

28. The method of any one of claims 25-27, wherein said pathogen is a coronavirus.

29. The method of claim 28, wherein said coronavirus is a betacoronavirus.

30. The method of claim 29, wherein said betacoronavirus is SARS-CoV-2.

31. The method of any one of claims 25-30, wherein said immunogen comprises a coronavirus Spike polypeptide or an immunogenic fragment thereof.

32. The method of any one of claims 25-31, wherein said mammal is a mammal that received said prior vaccine at least 90 days before said intranasally administering.

33. The method of any one of claims 25-35, wherein said mammal is a mammal that received said prior vaccine at least 120 days before said intranasally administering.

34. The method of any one of claims 25-31, wherein said mammal is a mammal that received said prior vaccine at least 150 days before said intranasally administering.

35. The method of any one of claims 25-31, wherein said mammal is a mammal that received said prior vaccine at least 180 days before said intranasally administering.

36. The method of any one of claims 256-35, wherein said prior vaccine was an mRNA-based vaccine.

37. The method of any one of claims 25-36, wherein said SC-Ad infects a cell of said mammal, and wherein expression of said immunogen in said cell leads to an increase in the amount of serum IgG antibodies that bind to said immunogen.

38. The method of any one of claims 25-37, wherein said SC-Ad infects a cell of said mammal, and wherein expression of said immunogen in said cell leads to an increase in the amount of mucosal IgA antibodies that bind to said immunogen.

39. The method of any one of claims 25-38, wherein said SC-Ad infects a cell of said mammal, and wherein expression of said immunogen in said cell leads to an increase in the amount of neutralizing antibodies in said human that bind to said immunogen.

40. The method of any one of claims 25-39, wherein said SC-Ad infects a cell of said mammal, and wherein expression of said immunogen in said cell leads to an increase in the number of Th1 T cells within said mammal that target said immunogen.

41. The method of claim 3, wherein said immunogen consists of or consists essentially of an amino acid sequence set forth in any one of SEQ ID NOs: 12, 42, 43, 44, 47, and 48 of International PCT Patent Publication No. WO 2022/040204.

42. A method for providing a human who received a prior vaccine against a virus causing respiratory disease with a vaccination boost, wherein said method comprises intranasally administering a single-cycle adenovirus (SC-Ad) to said human, wherein said SC-Ad comprises a genome lacking at least a portion of a nucleic acid sequence that encodes an adenovirus polypeptide, wherein said SC-Ad comprises said adenovirus polypeptide, and wherein said SC-Ad comprises a nucleic acid sequence encoding an immunogen of the virus causing respiratory disease.

43. The method of claim 42, wherein the virus causing the respiratory disease is selected from the group consisting of: a coronavirus; an influenza virus; a respiratory syncytial virus (RSV); a metapneumovirus (MPV); a rhinovirus; a bocavirus; and a parainfluenza virus (PIV).

44. The method of any one of claims 42-43, wherein said immunogen comprises a coronavirus Spike polypeptide or an immunogenic fragment thereof; an influenza haemagglutinin polypeptide; or an immunogenic fragment thereof, an RSV fusion (F) protein polypeptide, or an immunogenic fragment thereof, an MPV fusion (F) protein polypeptide, or an immunogenic fragment thereof, rhinovirus VP1 protein polypeptide, or an immunogenic fragment thereof, and a PIV fusion (F) protein polypeptide, or an immunogenic fragment thereof.

45. The method of claim 44, wherein said immunogen comprises a coronavirus Spike polypeptide or an immunogenic fragment thereof.

46. The method of claim 45, wherein said immunogen consists of or consists essentially of an amino acid sequence set forth in any one of SEQ ID NOs:1-4 of International PCT Patent Publication No. WO 2022/040204.

47. The method of claim 45, wherein said immunogen consists of or consists essentially of an amino acid sequence set forth in any one of SEQ ID NOs: 12, 42, 43, 44, 47, and 48 of International PCT Patent Publication No. WO 2022/040204.

48. The method of any one of claims 42-47, wherein said SC-Ad further comprises a nucleic acid sequence encoding an adjuvant polypeptide.

49. The method of claim 48, wherein said adjuvant polypeptide is selected from the group consisting of a granulocyte-macrophage colony-stimulating factor (GM-CSF) polypeptide, an interleukin 4 (IL-4) polypeptide, an interleukin 21 (IL-21) polypeptide, a CD40 ligand (CD40L) polypeptide, a 4-1BB ligand (4-1BBL) polypeptide, a transforming growth factor beta (TGF-β) polypeptide, a Clostridium difficile TcdA polypeptide, a C. difficile TcdB polypeptide, and biologically active fragments thereof.

50. The method of claim 48 or claim 49, wherein said immunogen is fused to said adjuvant polypeptide.

51. The method of claim 50, wherein said immunogen fused to said adjuvant polypeptide consists essentially of or consists of an amino acid sequence set forth in SEQ ID NO:5 of International PCT Patent Publication No. WO 2022/040204.

52. The method of any one of claims 42-47, wherein said SC-Ad further comprises a nucleic acid sequence encoding a chaff polypeptide.

53. The method of claim 52, wherein said chaff polypeptide is a fragment of an ACE2 polypeptide.

54. The method of claim 53, wherein said fragment of an ACE2 polypeptide comprises the extracellular region of an ACE2 polypeptide and lacks a transmembrane domain.

55. The method of claim 54, wherein said chaff polypeptide consists essentially of or consists of an amino acid sequence set forth in SEQ ID NO:8 of International PCT Patent Publication No. WO 2022/040204 or SEQ ID NO:9 of International PCT Patent Publication No. WO 2022/040204.

56. The method of any one of claims 52-55, wherein said immunogen is fused to said chaff polypeptide.

57. The method of any one of claims 42-56, wherein said human is a human who received said prior coronavirus vaccine at least 90 days before said intranasally administering.

58. The method of any one of claims 42-56, wherein said human is a human who received said prior coronavirus vaccine at least 120 days before said intranasally administering.

59. The method of any one of claims 42-56, wherein said human is a human who received said prior coronavirus vaccine at least 150 days before said intranasally administering.

60. The method of any one of claims 42-56, wherein said human is a human who received said prior coronavirus vaccine at least 180 days before said intranasally administering.

61. The method of any one of claims 42-60, wherein said prior vaccine was an mRNA-based vaccine.

62. The method of any one of claims 42-61, wherein said SC-Ad infects a cell of said human, and wherein expression of said immunogen in said cell leads to an increase in the amount of serum IgG antibodies that bind to said immunogen.

63. The method of any one of claims 42-62, wherein said SC-Ad infects a cell of said human, and wherein expression of said immunogen in said cell leads to an increase in the amount of mucosal IgA antibodies that bind to said immunogen.

64. The method of any one of claims 42-63, wherein said SC-Ad infects a cell of said human, and wherein expression of said immunogen in said cell leads to an increase in the amount of neutralizing antibodies in said human that bind to said immunogen.

65. The method of any one of claims 42-64, wherein said SC-Ad infects a cell of said human, and wherein expression of said immunogen in said cell leads to an increase in the number of Th1 T cells within said human that target said immunogen.

66. A method for providing a mammal who received a prior vaccine targeting a pathogen with a vaccination boost against said pathogen, wherein said method comprises intranasally administering a single-cycle adenovirus (SC-Ad) to said mammal, wherein said SC-Ad comprises a genome lacking at least a portion of a nucleic acid sequence that encodes an adenovirus polypeptide, wherein said SC-Ad comprises said adenovirus polypeptide, wherein said SC-Ad comprises a nucleic acid sequence encoding an immunogen of said pathogen.

67. The method of claim 66, wherein said mammal is a human.

68. The method of any one of claims 66-67, wherein the pathogen causes a respiratory disease.

69. The method of claim 68, wherein the pathogen causing the respiratory disease is selected from the group consisting of: a coronavirus; an influenza virus; a respiratory syncytial virus (RSV); a metapneumovirus (MPV); a rhinovirus; a bocavirus; and a parainfluenza virus (PIV).

70. The method of any one of claims 66-69, wherein said adenovirus polypeptide is selected from the group consisting of a fiber polypeptide, a V polypeptide, a hexon polypeptide, a penton base polypeptide, and a pIIIa polypeptide.

71. The method of any one of claims 66-70, wherein said mammal is a mammal that received said prior vaccine at least 90 days before said intranasally administering.

72. The method of any one of claims 66-71, wherein said mammal is a mammal that received said prior vaccine at least 120 days before said intranasally administering.

73. The method of any one of claims 66-71, wherein said mammal is a mammal that received said prior vaccine at least 150 days before said intranasally administering.

74. The method of any one of claims 66-71, wherein said mammal is a mammal that received said prior vaccine at least 180 days before said intranasally administering.

75. The method of any one of claims 66-74, wherein said prior vaccine was an mRNA-based vaccine.

76. The method of any one of claims 66-75, wherein said SC-Ad infects a cell of said mammal, and wherein expression of said immunogen in said cell leads to an increase in the amount of serum IgG antibodies that bind to said immunogen.

77. The method of any one of claims 66-76, wherein said SC-Ad infects a cell of said mammal, and wherein expression of said immunogen in said cell leads to an increase in the amount of mucosal IgA antibodies that bind to said immunogen.

78. The method of any one of claims 66-77, wherein said SC-Ad infects a cell of said mammal, and wherein expression of said immunogen in said cell leads to an increase in the amount of neutralizing antibodies in said human that bind to said immunogen.

79. The method of any one of claims 66-78, wherein said SC-Ad infects a cell of said mammal, and wherein expression of said immunogen in said cell leads to an increase in the number of Th1 T cells within said mammal that target said immunogen.
